(19) United States
(12) Patent Application Publication
(10) Pub. No.: US 2023/0329742 A1
(43) Pub. Date: Oct. 19, 2023

(54) APPLICATOR FOR SURGICAL INSTRUMENT LUBRICANT

(71) Applicant: Cilag GmbH International, Zug (CH)

(72) Inventors: Steven M. Boronyak, Cincinnati, OH (US); Demetrius N. Harris, Austin, TX (US); Patrick G. McElhaney, JR., Cincinnati, OH (US); Scott L. Johnson, Madison, WI (US); Elie R. F. Laumond, South Lebanon, OH (US); Johnnie Bell, Cincinnati, OH (US); Maxwell T. Rockman, Cincinnati, OH (US); Jacob S. Gee, Cincinnati, OH (US); Jacqueline A. Anim, Clayton, OH (US); John E. Brady, Liberty Township, OH (US); Anna Peterson Wilson, Cincinnati, OH (US); Oscar R. Chavez Montes, Cincinnati, OH (US); Jeffrey L. Aldridge, Lebanon, OH (US); Candice Otrembiak, Loveland, OH (US); Nicholas D. Schley, Cincinnati, OH (US); Stephen M. Torain, Cincinnati, OH (US); Chad P. Boudreaux, Cincinnati, OH (US); Kylie L. Hays, Cincinnati, OH (US); Rosa Castillo, Cincinnati, OH (US); Todd E. Riethmiller, Blue Ash, OH (US); Rachael A. Swenson, Cincinnati, OH (US); Louis P. Mingione, Madison, WI (US); Monica L. Rivard, Cincinnati, OH (US); Justin M. Kovach, Cincinnati, OH (US); Mark E. Tebbe, Lebanon, OH (US); Sean P. Conlon, Loveland, OH (US)

(21) Appl. No.: 17/721,407

(22) Filed: Apr. 15, 2022

(51) Int. Cl. A61B 17/32; A61B 18/14

(52) U.S. Cl. CPC A61B 17/320092; A61B 18/1445; A61B 2017/320094

(57) ABSTRACT

An apparatus configured for use with an end effector of a surgical instrument, comprising: (a) a housing, wherein the housing includes a first housing portion defining a first gap, wherein the first gap is configured to receive a jaw of the end effector; and (b) a lubricant application member positioned within the first gap, wherein the lubricant application member is configured to be loaded with a lubricant for applying the lubricant to a tissue clamping surface of the jaw when the jaw is received within the first gap.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Further examples of such devices and related concepts are disclosed in U.S. Pat. No. 6,500,176 entitled “Electrosurgical Systems and Techniques for Sealing Tissue,” issued Dec. 31, 2002, the disclosure of which is incorporated by reference herein, in its entirety; U.S. Pat. No. 8,939,974, entitled “Surgical Instrument Comprising First and Second Drive Systems Actuatable by a Common Trigger Mechanism,” issued Jan. 27, 2015, the disclosure of which is incorporated by reference herein, in its entirety; U.S. Pat. No. 8,888,809, entitled “Surgical Instrument with Jaw Member,” issued Nov. 18, 2014, the disclosure of which is incorporated by reference herein, in its entirety; U.S. Pat. No. 9,161,803, entitled “Motor Driven Electrosurgical Device with Mechanical and Electrical Feedback,” issued Oct. 20, 2015, the disclosure of which is incorporated by reference herein, in its entirety; U.S. Pat. No. 9,877,720, entitled “Control Features for Articulating Surgical Device,” issued Jan. 30, 2018, the disclosure of which is incorporated by reference herein, in its entirety; U.S. Pat. No. 9,545,253, entitled “Surgical Instrument with Contained Dual Helix Actuator Assembly,” issued Jan. 17, 2017, the disclosure of which is incorporated by reference herein, in its entirety; and U.S. Pat. No. 9,526,565, entitled “Electrosurgical Devices,” issued Dec. 27, 2016, the disclosure of which is incorporated by reference herein, in its entirety.

Some electrosurgical instruments include an end effector with at least one compliant feature. Examples of such instruments are described in U.S. Pat. No. 9,149,325, entitled “End Effector with Compliant Clamping Jaw,” issued Oct. 6, 2015, the disclosure of which is incorporated by reference herein, in its entirety; and U.S. Pat. No. 9,877,782, entitled “Electrosurgical Instrument End Effector with Compliant Electrode,” issued Jan. 30, 2018, the disclosure of which is incorporated by reference herein, in its entirety.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4A depicts a side elevational view of a handle assembly of the electrosurgical instrument of FIG. 1, where the end effector is in an open and unfired state, where a portion of the handle assembly is omitted for purposes of clarity;

FIG. 4B depicts a side elevational view of the handle assembly of FIG. 4A, where the end effector is in a closed and unfired state, where a portion of the handle assembly is omitted for purposes of clarity;

FIG. 4C depicts a side elevational view of the handle assembly of FIG. 4A, where the end effector is in a closed and fired state, where a portion of the handle assembly is omitted for purposes of clarity;

FIG. 5A depicts a cross-sectional side view of the end effector of FIG. 2, where the end effector is in the open and unfired state, taken along line 5-5 of FIG. 2;

FIG. 5B depicts a cross-sectional side view of the end effector of FIG. 2, where the end effector is in the closed and unfired state, taken along line 5-5 of FIG. 2;

FIG. 5C depicts a cross-sectional side view of the end effector of FIG. 2, where the end effector is in the closed and fired state, taken along line 5-5 of FIG. 2;

FIG. 6 depicts a perspective view of an exemplary lubricant applier cartridge including a lubricant application sponge that may be used to apply lubricant to the end effector of FIG. 2 and an excess lubricant removal cloth that may be used to remove excess lubricant from the end effector of FIG. 2;

FIG. 7 depicts a cross-sectional end view of the lubricant applier cartridge of FIG. 6, taken along line 7-7 of FIG. 6;

FIG. 8A depicts a perspective view of the end effector of FIG. 2 and the lubricant applier cartridge of FIG. 6, showing the end effector and the lubricant application sponge of the lubricant applier cartridge being aligned with one another;

FIG. 8B depicts a perspective view of the end effector of FIG. 2 and the lubricant applier cartridge of FIG. 6, with the end effector jaws closed on the lubricant application sponge of the lubricant applier cartridge;

FIG. 8C depicts a perspective view of the end effector of FIG. 2 and the lubricant applier cartridge of FIG. 6, showing the end effector and the excess lubricant removal cloth of the lubricant applier cartridge being aligned with one another;

FIG. 8D depicts a perspective view of the end effector of FIG. 2 and the lubricant applier cartridge of FIG. 6, with the end effector jaws closed on the excess lubricant removal cloth of the lubricant applier cartridge;

FIG. 9 depicts a top plan view of another exemplary lubricant applier cartridge including a plurality of lubricant application sponges that may be used to apply lubricant to various types of end effectors including that of FIG. 2 and a plurality of excess lubricant removal cloths that may be used to remove excess lubricant from various types of end effectors including that of FIG. 2;

FIG. 10 depicts a top plan view of another exemplary lubricant applier cartridge including a plurality of lubricant application sponges that may be used to apply lubricant to various types of end effectors including that of FIG. 2 and a single excess lubricant removal cloth that may be used to remove excess lubricant from various types of end effectors including that of FIG. 2;

FIG. 11 depicts a perspective view of another exemplary lubricant applier cartridge including bonded lubricant application and excess lubricant removal sponges that may be used to apply lubricant to and remove excess lubricant from the end effector of FIG. 2;

FIG. 12A depicts a cross-sectional side view of the lubricant applier cartridge of FIG. 11, taken along line 12A-12A of FIG. 11, showing the end effector of FIG. 2 and the lubricant applier cartridge being aligned with one another;

FIG. 12B depicts a cross-sectional side view of the lubricant applier cartridge of FIG. 11, with the end effector jaws closed on both the lubricant application and excess lubricant removal sponges of the lubricant applier cartridge;

FIG. 13 depicts a cross-sectional side view of another exemplary lubricant applier cartridge including layered lubricant application and excess lubricant removal sponges that may be used to apply lubricant to and remove excess lubricant from the end effector of FIG. 2, showing the end effector and the lubricant applier cartridge being aligned with one another;

FIG. 14 depicts a cross-sectional side view of another exemplary lubricant applier cartridge including interlocked lubricant application and excess lubricant removal sponges that may be used to apply lubricant to and remove excess lubricant from the end effector of FIG. 2, showing the end effector and the lubricant applier cartridge being aligned with one another;

FIG. 15 depicts a perspective view of another exemplary lubricant applier cartridge including a jaw cleaning abrasive that may be used to clean the jaws of the end effector of FIG. 2, a lubricant application sponge that may be used to apply lubricant to the end effector of FIG. 2, and an excess lubricant removal cloth that may be used to remove excess lubricant from the end effector of FIG. 2;

FIG. 16 depicts a top plan view of the lubricant applier cartridge of FIG. 15;

FIG. 17 depicts a perspective view of another exemplary lubricant applier cartridge including a jaw cleaning abrasive that may be used to clean the jaws of the end effector of FIG. 2 and a lubricant application sponge that may be used to apply lubricant to the end effector of FIG. 2, and further including lubricant and cleaning solution injection ports for receiving respective syringes;

FIG. 18 depicts an exploded side elevational view of one of the syringes of FIG. 17;

FIG. 19 depicts a perspective view of another exemplary lubricant applier cartridge including a lubricant application sponge that may be used to apply lubricant to the end effector of FIG. 2, and further including a lubricant discharge button;

FIG. 20A depicts a cross-sectional side view of the lubricant applier cartridge of

FIG. 19, taken along line 20A-20A of FIG. 19, showing the lubricant discharge button in an unactuated state;

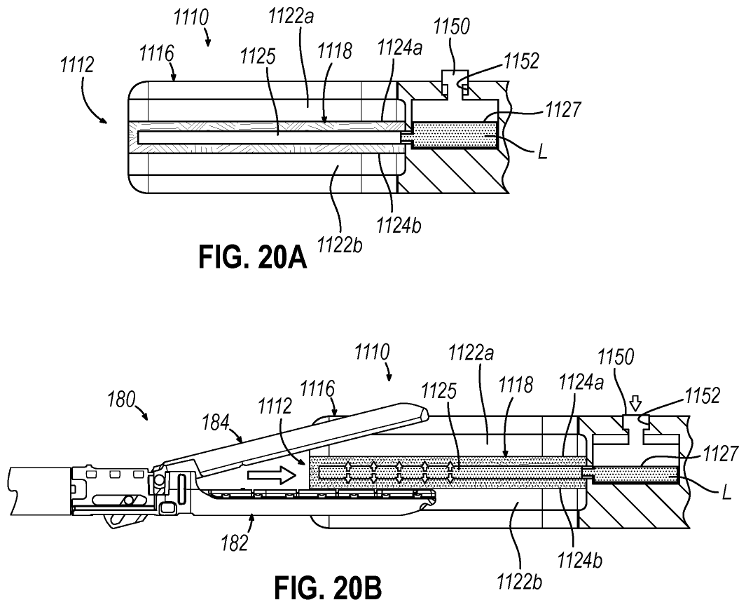

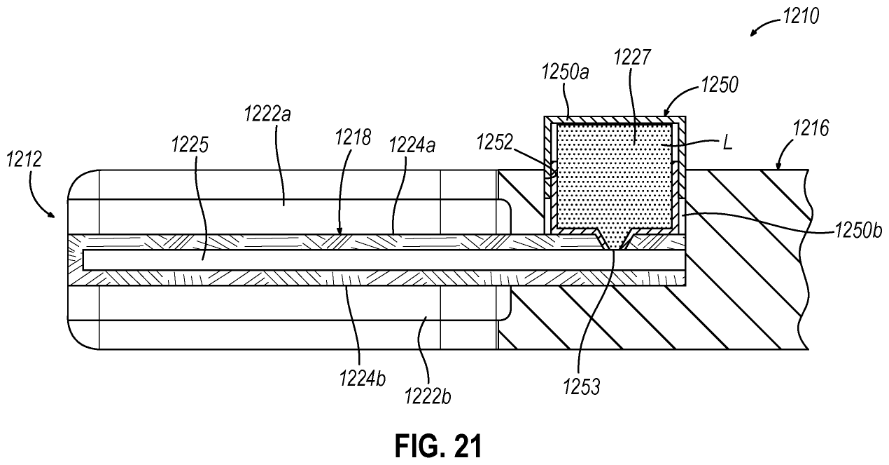

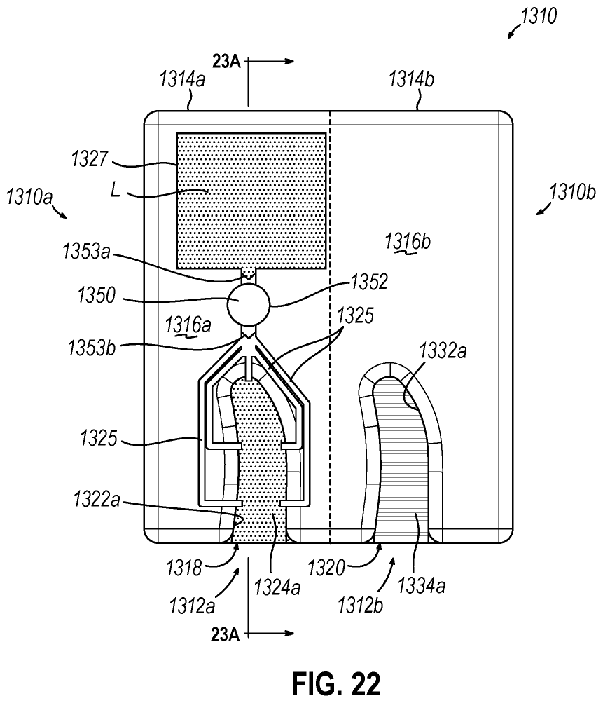

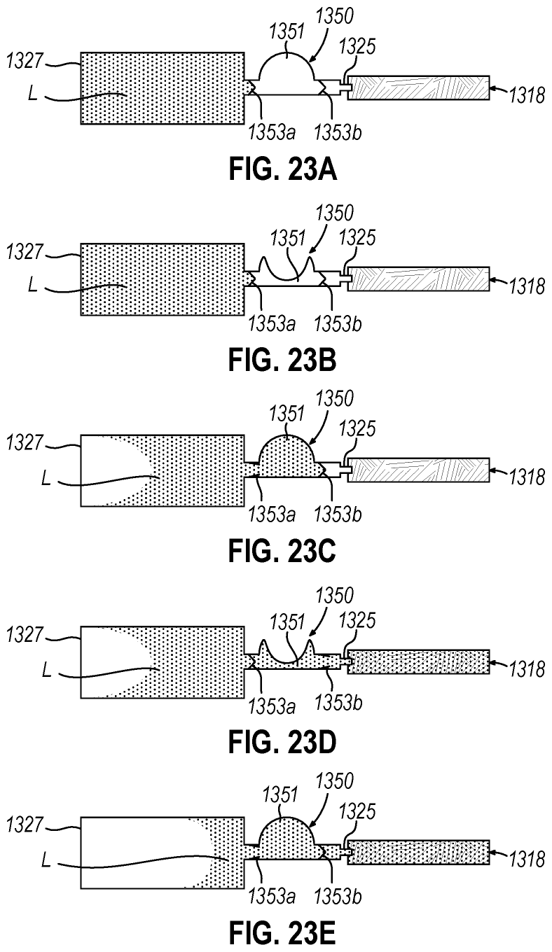

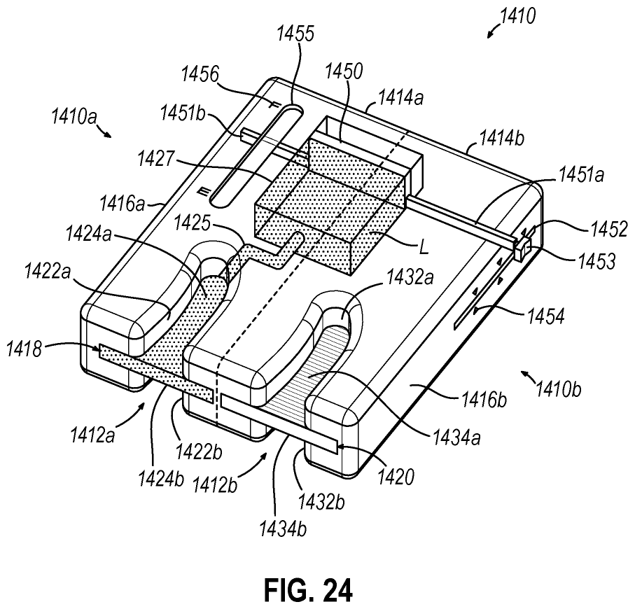

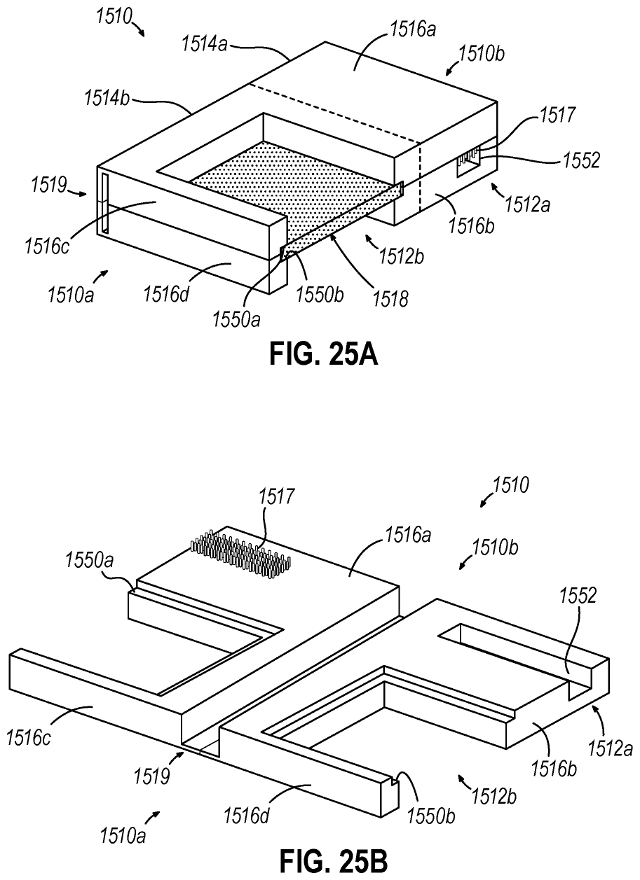

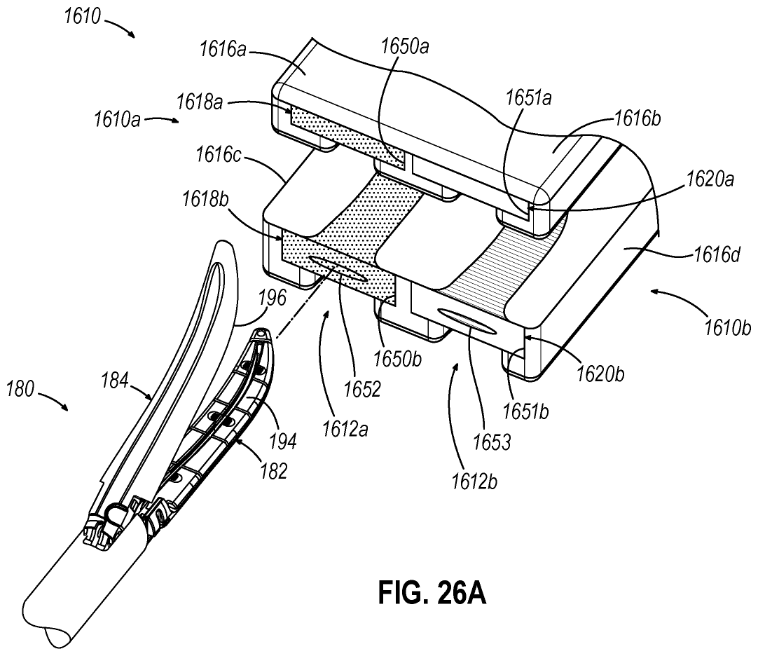

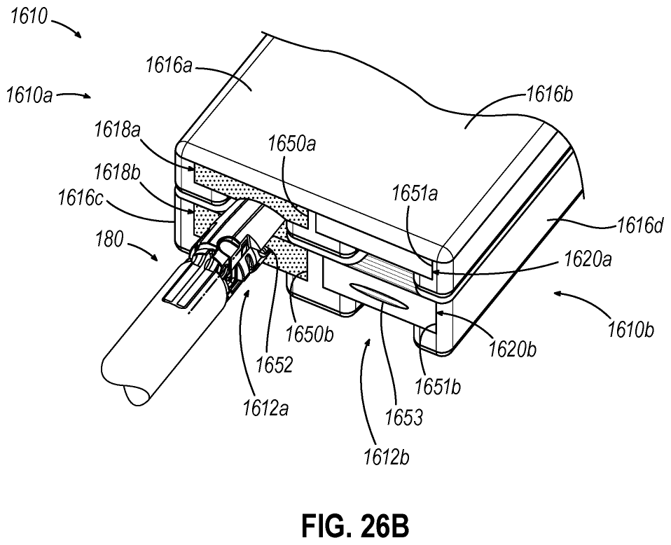

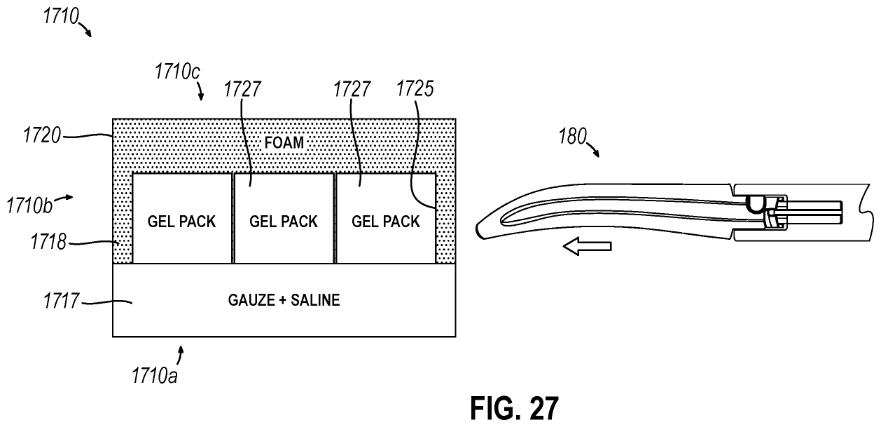

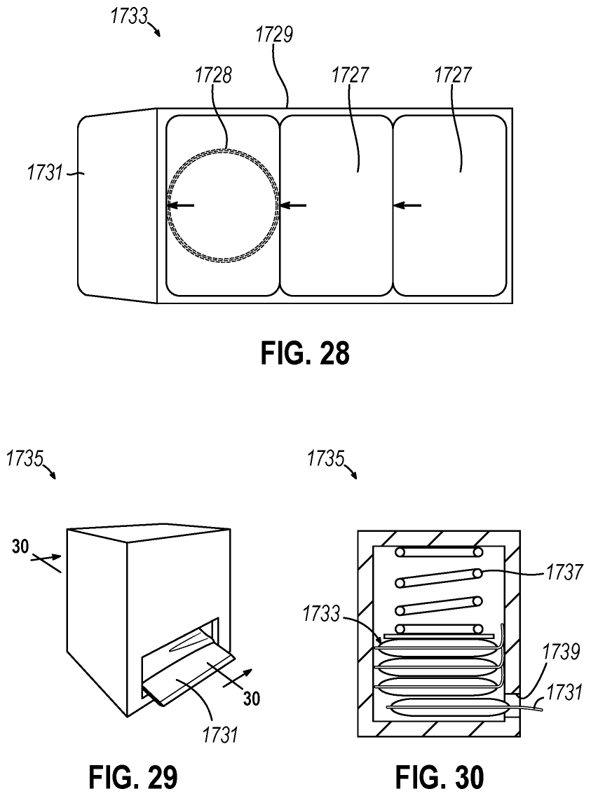

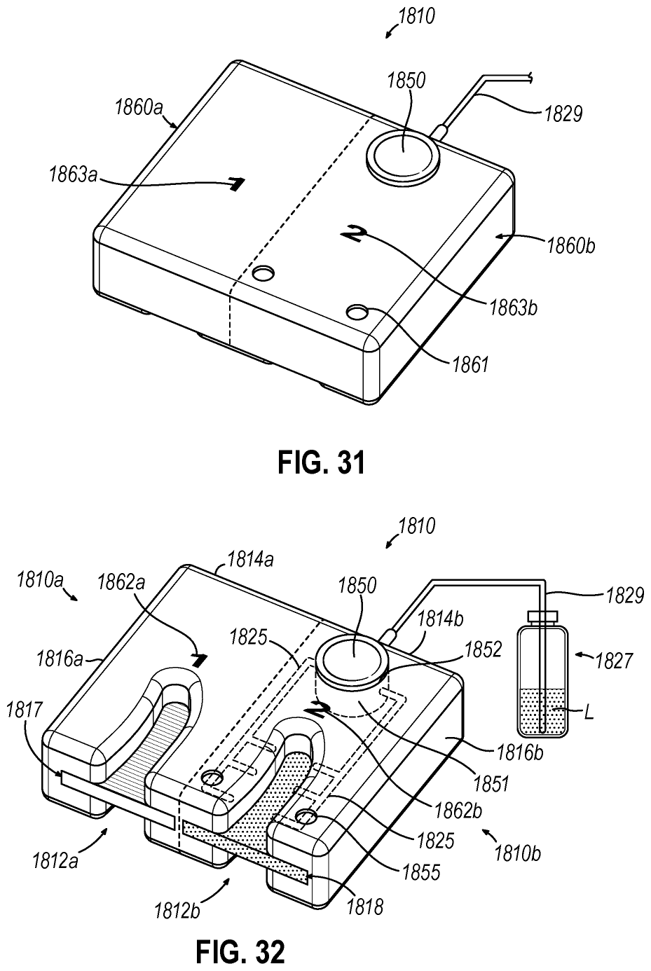

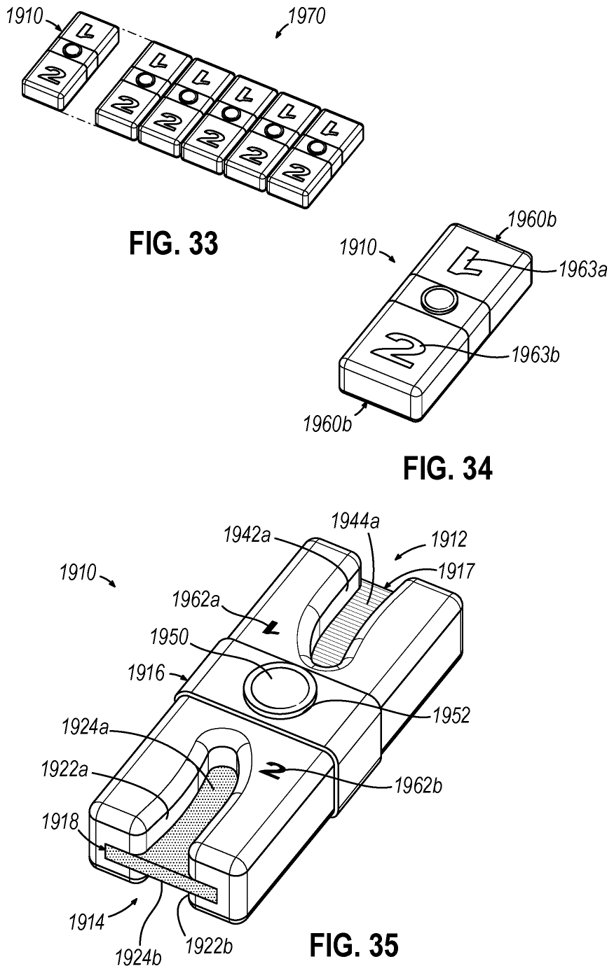

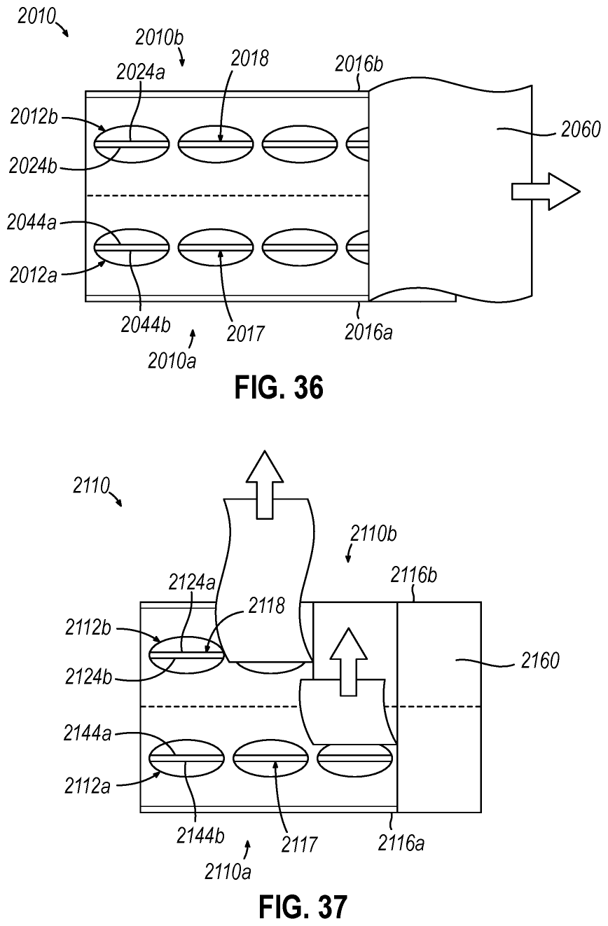

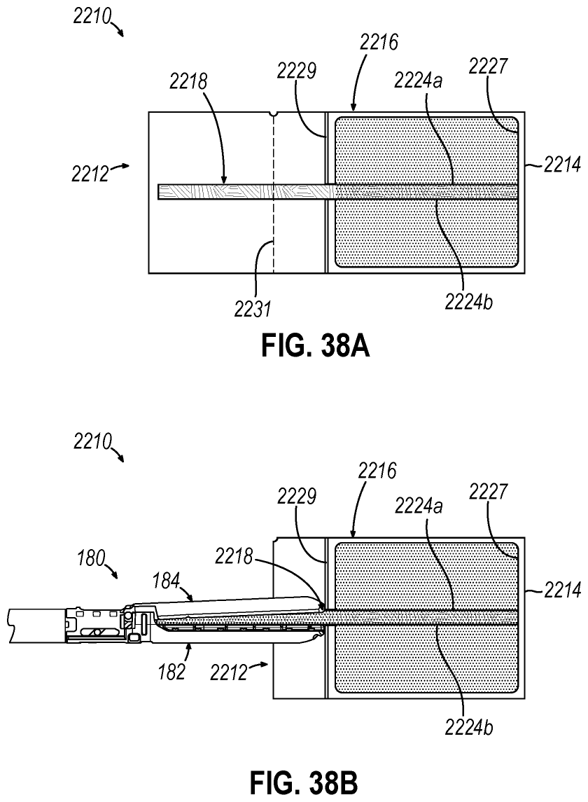

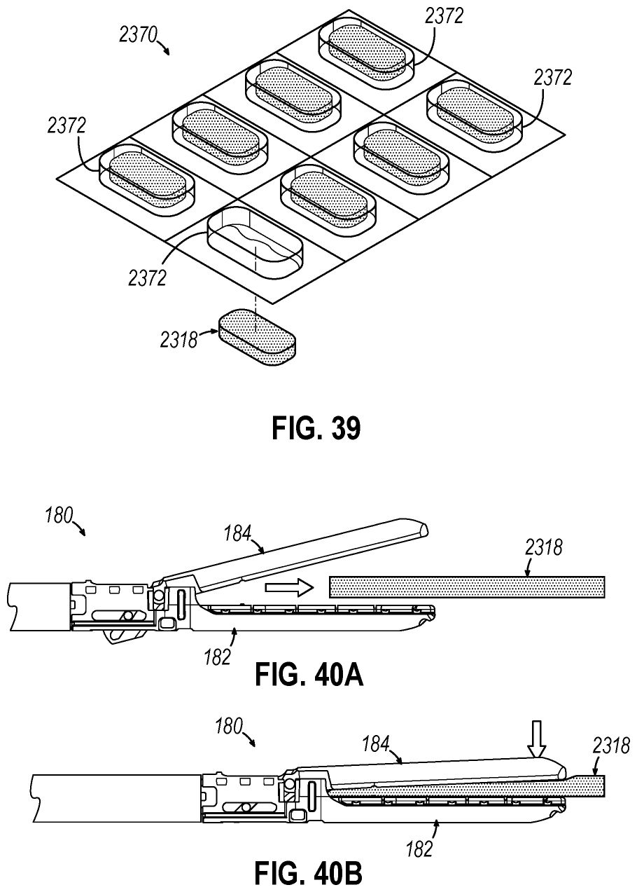

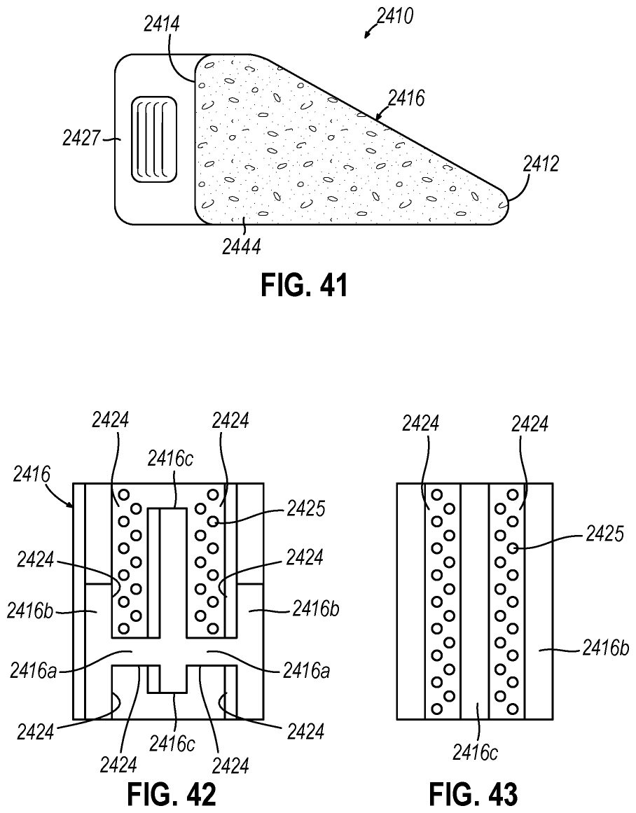

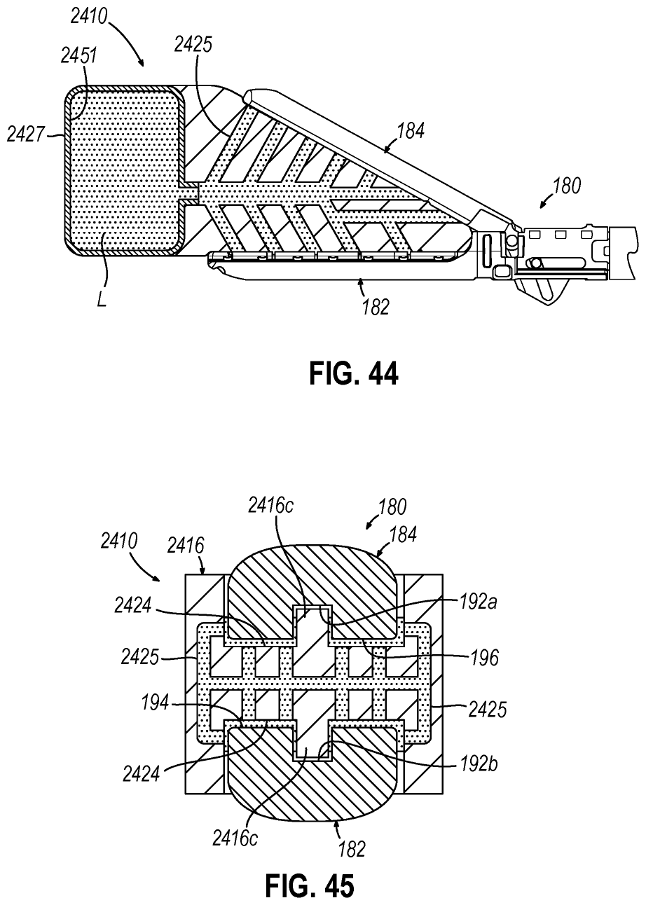

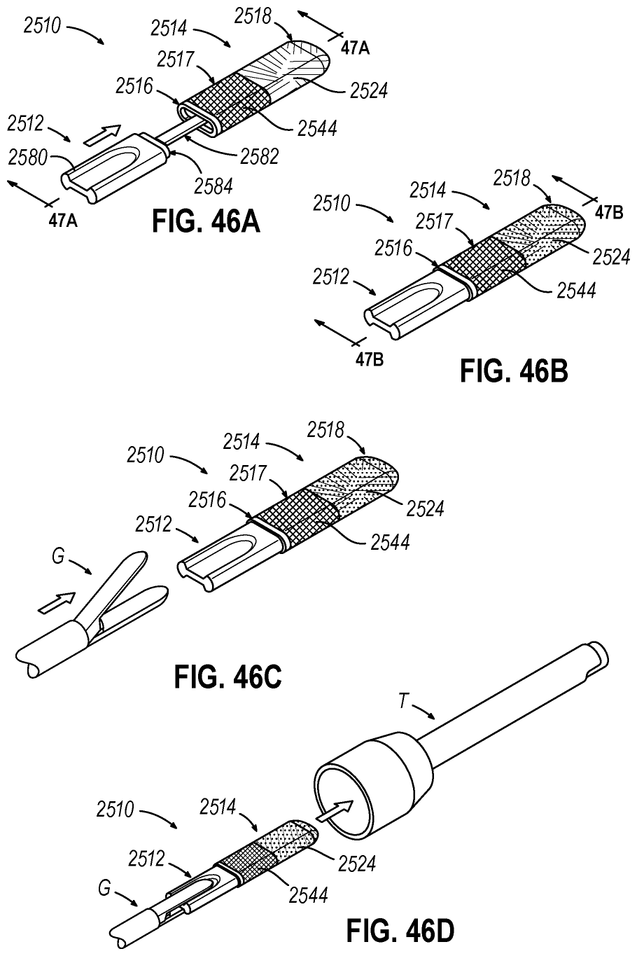

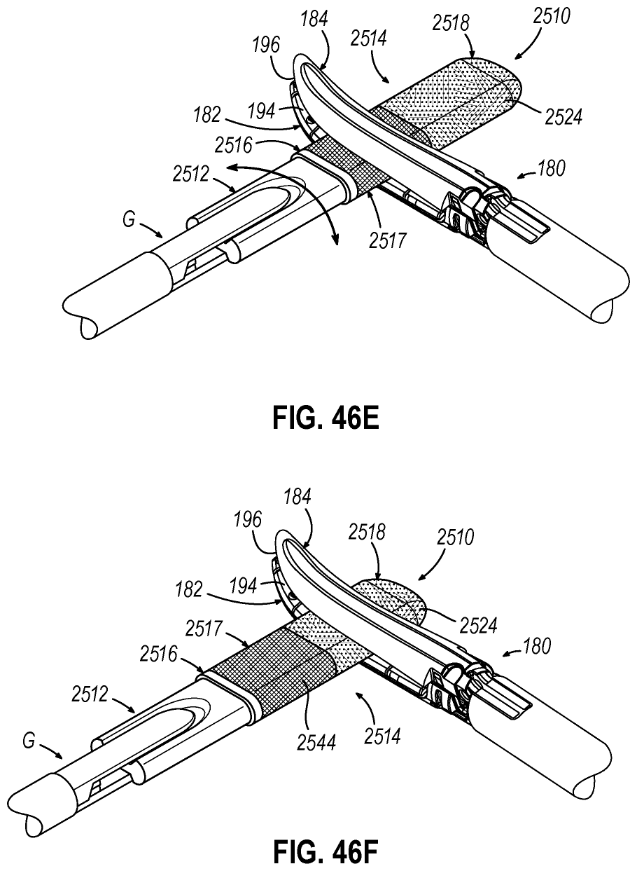

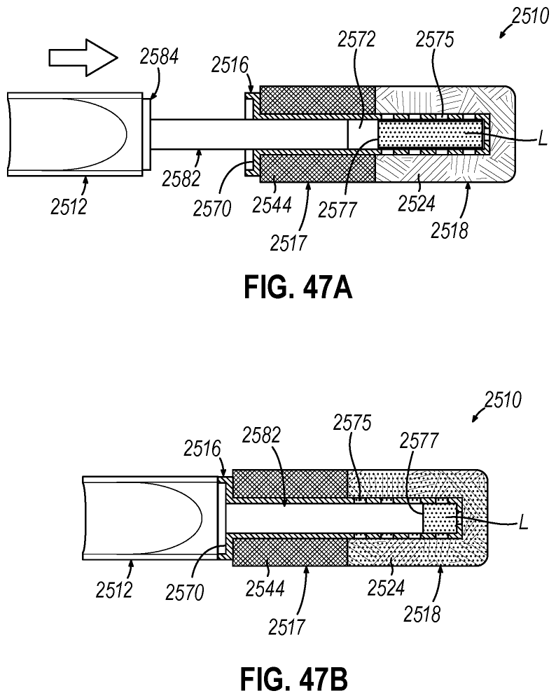

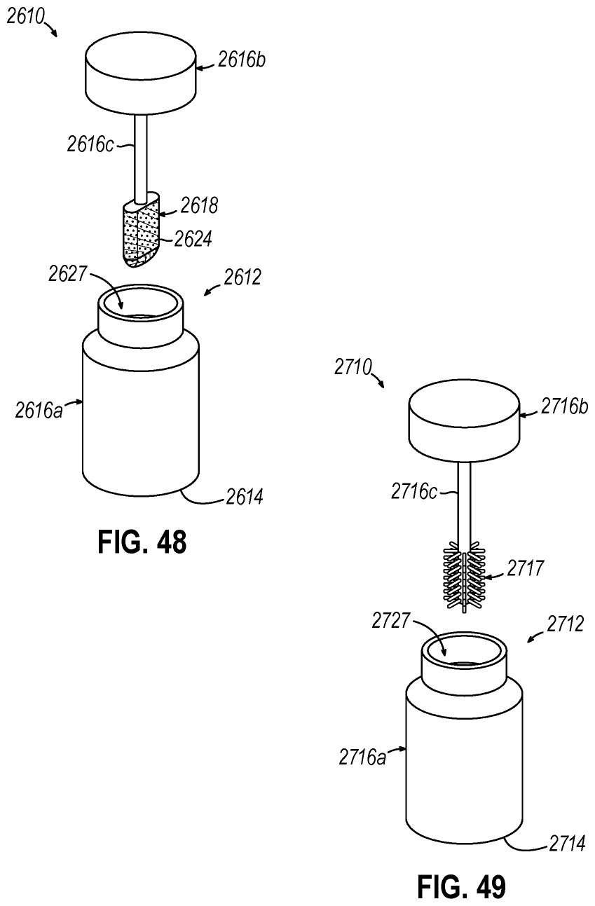

FIG. 20B depicts a cross-sectional side view of the lubricant applier cartridge of FIG. 19, showing the lubricant discharge button depressed to an actuated state to dispense lubricant from a rupturable pod into an internal channel of the lubricant application sponge for loading the lubricant application sponge with the lubricant, and further showing the end effector and the lubricant applier cartridge being aligned with one another;

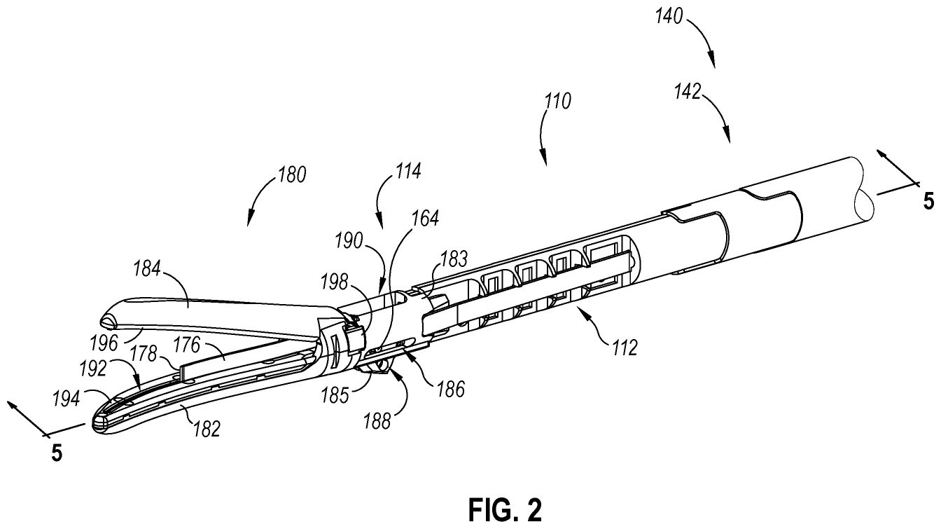

FIG. 21 depicts a cross-sectional side view of another exemplary lubricant applier cartridge including a lubricant application sponge that may be used to apply lubricant to the end effector of FIG. 2, and further including a lubricant discharge button with a one-way valve;

FIG. 22 depicts a top plan view of another exemplary lubricant applier cartridge including a lubricant application sponge that may be used to apply lubricant to the end effector of FIG. 2 and an excess lubricant removal cloth that may be used to remove excess lubricant from the end effector of FIG. 2, and further including a lubricant discharge pump bulb;

FIG. 23A depicts a partial cross-sectional side view of the lubricant applier cartridge of FIG. 22, taken along line 23A-23A of FIG. 22, showing the lubricant discharge pump bulb in an unactuated state;

FIG. 23B depicts a partial cross-sectional side view of the lubricant applier cartridge of FIG. 22, showing the lubricant discharge pump bulb depressed to an actuated state to create a vacuum within the lubricant discharge pump bulb;

FIG. 23C depicts a partial cross-sectional side view of the lubricant applier cartridge of FIG. 22, showing the lubricant discharge pump bulb returned the unactuated state to fill the lubricant discharge pump bulb with lubricant from an internal compartment of the lubricant applier cartridge;

FIG. 23D depicts a partial cross-sectional side view of the lubricant applier cartridge of FIG. 22, showing the lubricant discharge pump bulb depressed to the actuated state to direct the lubricant from the lubricant discharge pump bulb into at least one internal dispersion channel of the lubricant applier cartridge for loading the lubricant application sponge with the lubricant;

FIG. 23E depicts a partial cross-sectional side view of the lubricant applier cartridge of FIG. 22, showing the lubricant discharge pump bulb returned to the unactuated state to again fill the lubricant discharge pump bulb with lubricant from the internal compartment of the lubricant applier cartridge;

FIG. 24 depicts a perspective view of another exemplary lubricant applier cartridge including a lubricant application sponge that may be used to apply lubricant to the end effector of FIG. 2 and an excess lubricant removal cloth that may be used to remove excess lubricant from the end effector of FIG. 2, and further including a lubricant discharge paddle;

FIG. 25A depicts a perspective view of another exemplary lubricant applier cartridge including a jaw cleaning brush that may be used to clean the jaws of the end effector of FIG. 2 and a lubricant application sponge that may be used to apply lubricant to the end effector of FIG. 2, showing the lubricant applier cartridge in a closed state;

FIG. 25B depicts a perspective view of the lubricant applier cartridge of FIG. 25A, showing the lubricant applier cartridge in an open state;

FIG. 26A depicts a perspective view of another exemplary lubricant applier cartridge including upper and lower lubricant application sponges that may be used to apply lubricant to the end effector of FIG. 2 and upper and lower excess lubricant removal sponges that may be used to remove excess lubricant from the end effector of FIG. 2, showing the lubricant applier cartridge in an open state, and further showing the end effector and the lubricant application sponges of the lubricant applier cartridge being aligned with one another;

FIG. 26B depicts a perspective view of the lubricant applier cartridge of FIG. 26A, with the end effector jaws closed on the lower lubricant application sponge of the lubricant applier cartridge, and showing the lubricant applier cartridge in a closed state;

FIG. 27 depicts a schematic view of another exemplary lubricant applier cartridge including a jaw cleaning gauze strip that may be used to clean the jaws of the end effector of FIG. 2, a lubricant application sponge that may be used to apply lubricant to the end effector of FIG. 2, and an excess lubricant removal cloth that may be used to remove excess lubricant from the end effector of FIG. 2, and further showing the end effector and the lubricant application sponge of the lubricant applier cartridge being aligned with one another;

FIG. 28 depicts a top plan view of an exemplary lubricant reload for use with the lubricant applier cartridge of FIG. 27;

FIG. 29 depicts a perspective view of a dispenser for containing a supply of the lubricant reloads of FIG. 28;

FIG. 30 depicts a cross-sectional view of the dispenser of FIG. 29, taken along line 30-30 of FIG. 29;

FIG. 31 depicts a perspective view of another exemplary lubricant applier cartridge including a jaw cleaning abrasive that may be used to clean the jaws of the end effector of FIG. 2 and a lubricant application sponge that may be used to apply lubricant to the end effector of FIG. 2, and further including a lubricant discharge pump bulb, with respective caps selectively covering the jaw cleaning abrasive and the lubricant application sponge;

FIG. 32 depicts a perspective view of the lubricant applier cartridge of FIG. 31, with the caps removed and with the lubricant discharge pump bulb fluidly coupled to an external bottle to direct the lubricant from the external bottle into internal dispersion channels of the lubricant applier cartridge for loading the lubricant application sponge with the lubricant;

FIG. 33 depicts a perspective view of an exemplary multi-pack of lubricant applier cartridges frangibly coupled to each other, each including a jaw cleaning abrasive that may be used to clean the jaws of the end effector of FIG. 2 and a lubricant application sponge that may be used to apply lubricant to the end effector of FIG. 2, and further including a lubricant discharge pump bulb;

FIG. 34 depicts a perspective view of one of the lubricant applier cartridges of FIG. 33, with respective caps selectively covering the jaw cleaning abrasive and the lubricant application sponge;

FIG. 35 depicts a perspective view of the lubricant applier cartridge of FIG. 34, with the caps removed;

FIG. 36 depicts a top plan view of another exemplary lubricant applier cartridge including a plurality of jaw cleaning abrasives disposed within respective wells that may be used to clean the jaws of the end effector of FIG. 2 and a plurality of lubricant application sponges disposed within respective wells that may be used to apply lubricant to the end effector of FIG. 2, showing removal of a single peelable lid from the lubricant applier cartridge;

FIG. 37 depicts a top plan view of another exemplary lubricant applier cartridge including a plurality of jaw cleaning abrasives disposed within respective wells that may be used to clean the jaws of the end effector of FIG. 2 and a plurality of lubricant application sponges disposed within respective wells that may be used to apply lubricant to the end effector of FIG. 2, showing selective removal of various discrete peelable lids from the lubricant applier cartridge;

FIG. 38A depicts a cross-sectional side view of an exemplary lubricant applier pouch including a lubricant application sponge that may be used to apply lubricant to the end effector of FIG. 2, showing an openable end of the lubricant applier pouch in a closed state;

FIG. 38B depicts a cross-sectional side view of the lubricant applier pouch of FIG. 38A, showing the openable end of the lubricant applier pouch in an opened state, with the end effector jaws closed on the lubricant application sponge of the lubricant applier pouch;

FIG. 39 depicts an exemplary blister pack containing a plurality of individually packaged, pre-loaded lubricant applier sponges that may be used to apply lubricant to the end effector of FIG. 2;

FIG. 40A depicts a side elevational view of one of the lubricant applier sponges of FIG. 39, showing the end effector of FIG. 2 and the lubricant applier sponge being aligned with one another;

FIG. 40B depicts a side elevational view of the lubricant applier sponge of FIG. 40A, with the end effector jaws closed on the lubricant applier sponge;

FIG. 41 depicts a side elevational view of an exemplary lubricant applier wedge including a jaw cleaning surface that may be used to clean the jaws of the end effector of FIG. 2 and a plurality of lubricant application surfaces that may be used to apply lubricant to the end effector of FIG. 2;

FIG. 42 depicts a front elevational view of the lubricant applier wedge of FIG. 41;

FIG. 43 depicts a bottom plan view of the lubricant applier wedge of FIG. 41;

FIG. 44 depicts a cross-sectional side view of the lubricant applier wedge of FIG. 41, with the wedge received between the open end effector jaws;

FIG. 45 depicts a cross-sectional end view of the lubricant applier wedge of FIG. 41, with the wedge received between the open end effector jaws;

FIG. 46A depicts a perspective view of an exemplary laparoscopic lubricant applier assembly having a plunger and a capsule including a jaw cleaning gauze strip that may be used to clean the jaws of the end effector of FIG. 2 and a lubricant application sponge that may be used to apply lubricant to the end effector of FIG. 2, showing the plunger in a proximal state;

FIG. 46B depicts a perspective view of the laparoscopic lubricant applier assembly of FIG. 46A, showing the plunger in a distal state to dispense the lubricant from a rupturable pod into outlets of the capsule for loading the lubricant application sponge with the lubricant;

FIG. 46C depicts a perspective view of the laparoscopic lubricant applier assembly of FIG. 46A, showing a grasper and a grip of the plunger being aligned with one another;

FIG. 46D depicts a perspective view of the laparoscopic lubricant applier assembly of FIG. 46A, showing the laparoscopic lubricant applier assembly being inserted through a trocar via the grasper;

FIG. 46E depicts a perspective view of the laparoscopic lubricant applier assembly of FIG. 46A, with the end effector jaws closed on the jaw cleaning gauze strip of the capsule, showing the laparoscopic lubricant applier assembly being rotated between the end effector jaws via the grasper;

FIG. 46F depicts a perspective view of the laparoscopic lubricant applier assembly of FIG. 46A, with the end effector jaws closed on the lubricant application sponge of the capsule;

FIG. 47A depicts a cross-sectional side view of the laparoscopic lubricant applier assembly of FIG. 46A, taken along line 47A-47A of FIG. 46A, showing the plunger in the proximal state;

FIG. 47B depicts a cross-sectional side view of the laparoscopic lubricant applier assembly of FIG. 46A, taken along line 47B-47B of FIG. 46B, showing the plunger in the distal state;

FIG. 48 depicts a perspective view of an exemplary lubricant applier bottle including a lubricant application sponge that may be used to apply lubricant to the end effector of FIG. 2; and

FIG. 49 depicts a perspective view of an exemplary cleaning solution applier bottle including a jaw cleaning brush that may be used to clean the jaws of the end effector of FIG. 2.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

I. Example of Electrosurgical Instrument

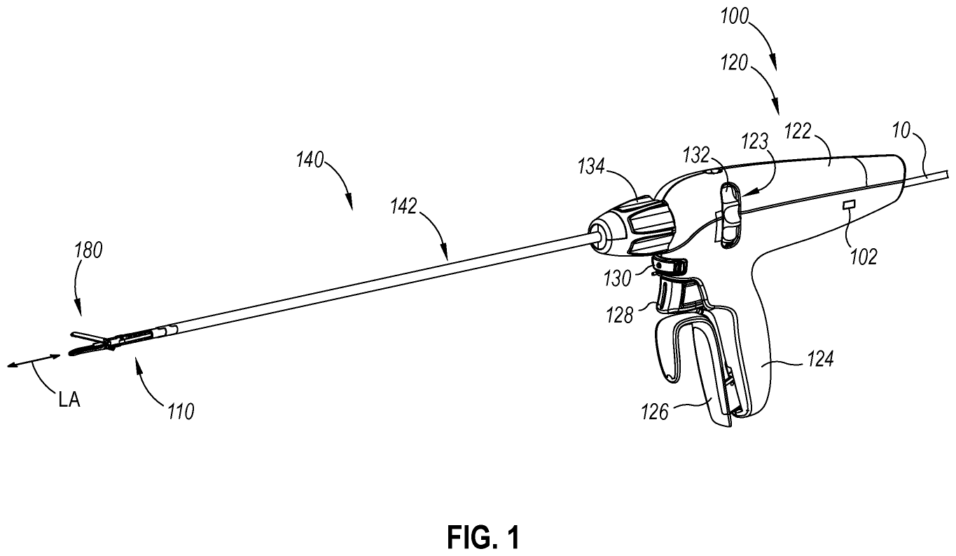

FIGS. 1-3C show an exemplary electrosurgical instrument (100). As best seen in FIG. 1, electrosurgical instrument (100) includes a handle assembly (120), a shaft assembly (140), an articulation assembly (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to seal or weld tissue by applying bipolar radio frequency (RF) energy to tissue. However, it should be understood electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a power source (not shown) via power cable (10).

The power source may be configured to provide all or some of the electrical power requirements for use of electrosurgical instrument (100). Any suitable power source may be used as would be apparent to one skilled in the art in view of the teachings herein. By way of example only, the power source may comprise a GEN04 or GEN11 sold by Ethicon Endo-Surgery, Inc. of Cincinnati, Ohio. In addition, or in the alternative, the power source may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to a power source via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrode surfaces (194, 196) of jaws (182, 184), respectively.

Body (122) of handle assembly (120) defines an opening (123) in which a portion of articulation control (132) protrudes from. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) is configured to bend articulation section (110) in order to drive deflection of end effector (180) from the longitudinal axis (LA) defined by shaft assembly (140). Articulation control (132) and articulation section (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

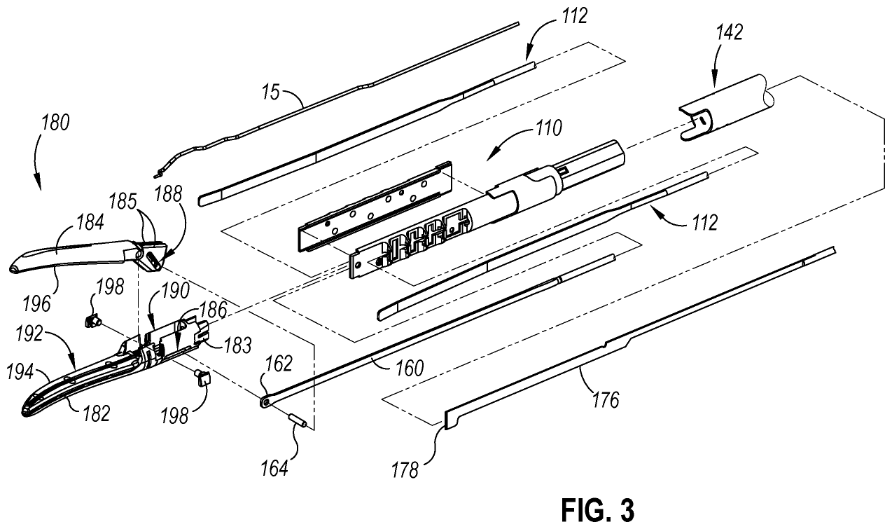

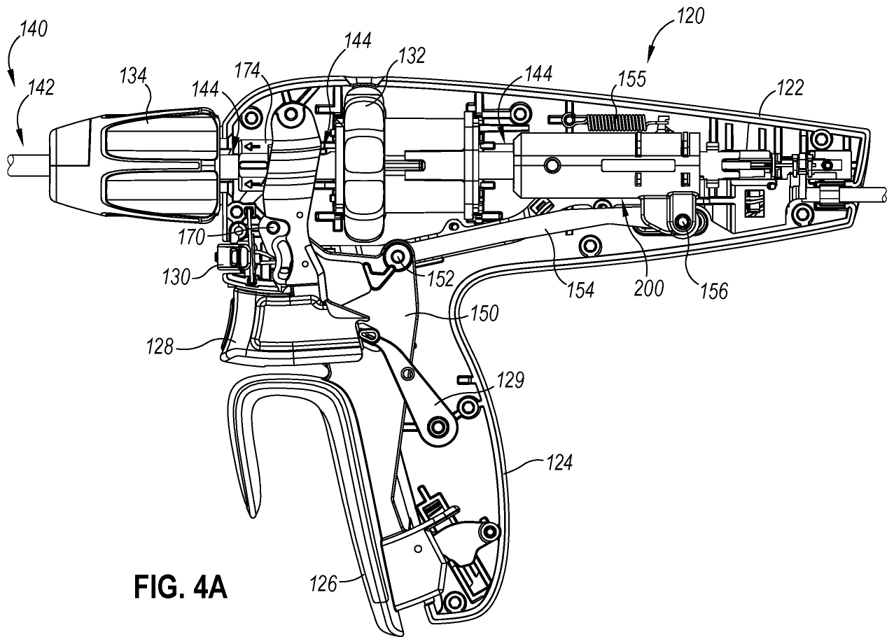

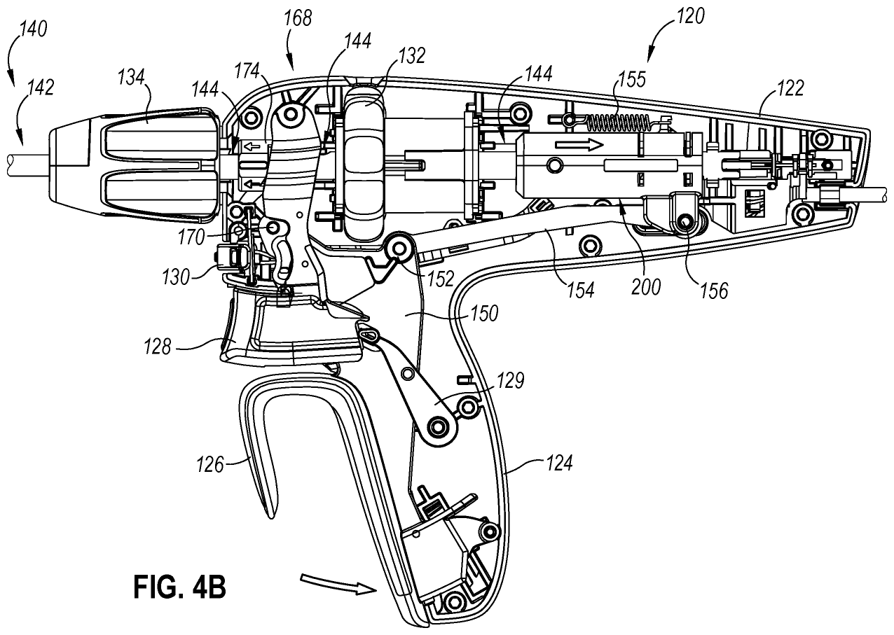

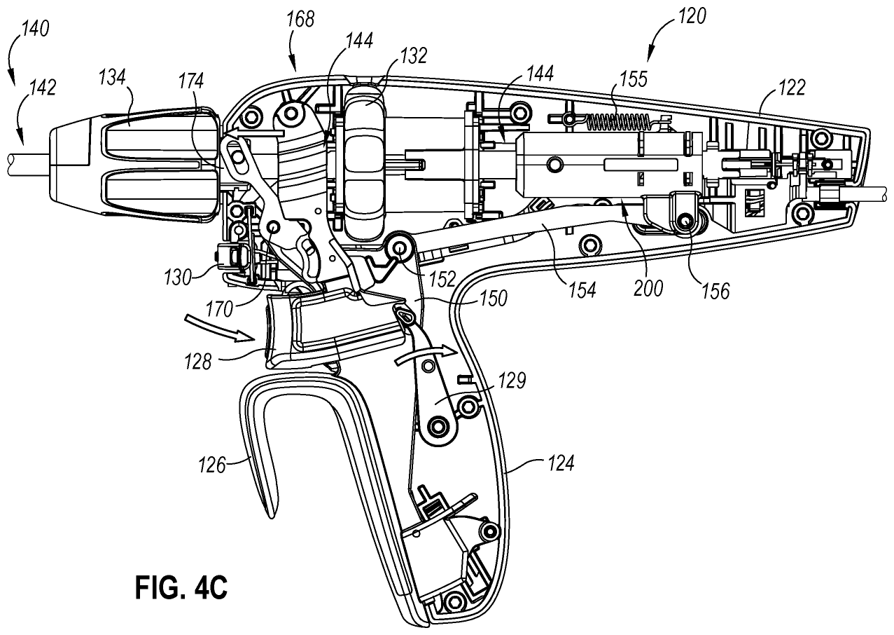

Shaft assembly (140) includes distal portion (142) extending distally from handle assembly (120), and a proximal portion (144) (see FIGS. 4A-4B) housed within the confines of body (122) of handle assembly (120). As best shown in FIG. 3, shaft assembly (140) houses a jaw closure connector (160) that couples jaw closure trigger (126) with end effector (180). Additionally, shaft assembly (140) houses a portion of knife member extending between distal cutting edge (178) and knife trigger (128). Shaft assembly (140) also houses actuating members (112) that couple articulation assembly (110) with articulation control (132); as well as an electrical connecter (15) that operatively couples electrode surfaces (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrode surface (194, 196).

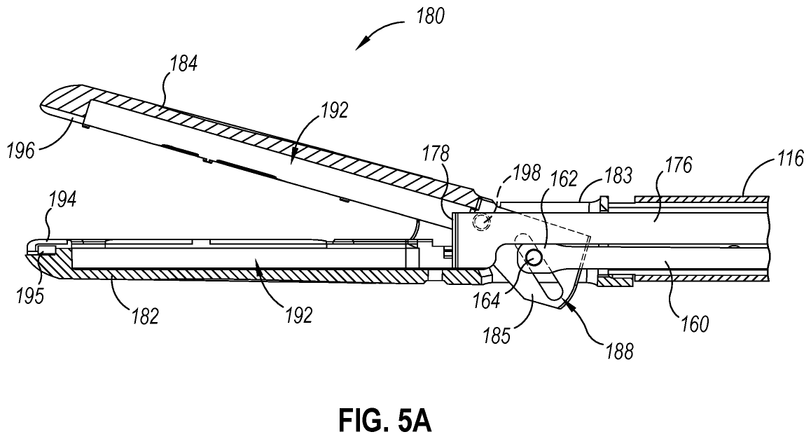

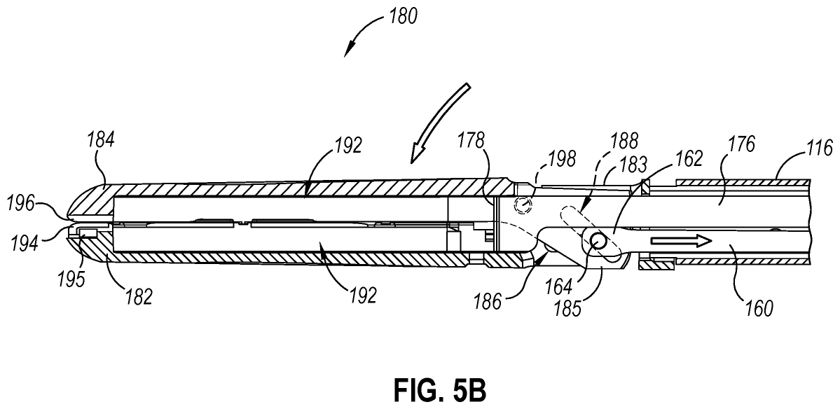

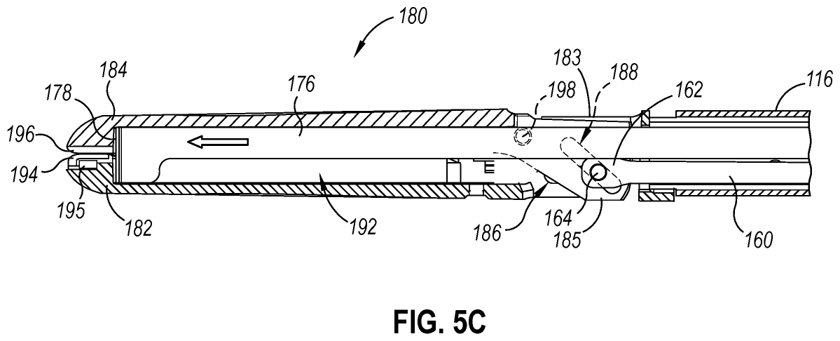

As best seen in FIGS. 2-3, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connecter (160), and pin (164). Slots (186, 188) each slidably receive pin (164), which is attached to a distal coupling portion (162) of jaw closure connector (160). Additionally, as best seen in FIGS. 5A-5C, lower jaw (182) includes a force sensor (195) located at a distal tip of lower jaw (182). Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge.

While in the current example, a force sensor (195) is incorporated into instrument (100) and is in communication with control unit (102), any other suitable sensors or feedback mechanisms may be additionally or alternatively incorporated into instrument (100) while in communication with control unit (102) as would be apparent to one skilled in the art in view of the teachings herein. For instance, an articulation sensor or feedback mechanism may be incorporated into instrument (100), where the articulation sensor communicates signals to control unit (102) indicative of the degree end effector (180) is deflected from the longitudinal axis (LA) by articulation control (132) and articulation section (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted (as shown in FIGS. 5A-5B), and advanced (as shown in FIG. 5C), to cut tissue captured between jaws (182, 184). Lower jaw (182) and upper jaw (184) each comprise a respective electrode surface (194, 196). The power source may provide RF energy to electrode surfaces (194, 196) via electrical coupling (15) that extends through handle assembly (120), shaft assembly (140), articulation assembly (110), and electrically couples with one or both of electrode surfaces (194, 196). Electrical coupling (15) may selectively activate electrode surfaces (194, 196) in response to an operator pressing activation button (130). In some instances, control unit (102) may couple electrical coupling (15) with activation button (130), such that control unit (102) activates electrode surfaces (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc.

FIGS. 4A-5C show an exemplary use of instrument (100) for end effector (180) to grasp, cut, and seal/weld tissue. As described above, and as shown between FIGS. 4A-4B and 5A-5B, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

Handle assembly (120) further includes a yoke assembly (200) that is slidably coupled along proximal portion (144) of shaft assembly (140). Yoke assembly (200) is operatively coupled with jaw closure connector (160) such that translation of yoke assembly (200) relative to proximal portion (144) of shaft assembly (140) translates jaw closure connector (160) relative to shaft assembly (140).

As best seen in FIGS. 4A-4C, yoke assembly (200) is coupled to a body (150) of jaw closure trigger (126) via a link (154). Link (154) is pivotally coupled with yoke assembly (200) via pin (156); while link (154) is also pivotally coupled with body (150) of jaw closure trigger (126) via pin (152). Additionally, jaw closure trigger (126) is pivotally coupled with body (122) of handle assembly (120) via pin (170). Therefore, as shown between FIGS. 4A-4B, an operator may pull jaw closure trigger (126) toward pistol grip (124), thereby rotating jaw closure trigger (126) about pin (170). Rotation of jaw closure trigger (126) leads to rotation of link (154) about both pins (152, 156), which in turn drives yoke assembly (200) in the proximal direction along proximal portion (144) of shaft assembly (140).

As described above, jaw closure connector (160) extends within shaft assembly (140), articulation section (110), and central channel (190) of lower jaw (182). As also mentioned above, jaw closure connector (160) is attached to pin (164). Therefore, as seen between FIGS. 5A-5B, proximal translation of yoke assembly (200) leads to proximal translation of pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

As best seen in FIGS. 4A-4C, yoke assembly (200) is also coupled with a bias spring (155). Bias spring (155) is also coupled to a portion of body (122), such that bias spring (155) biases yoke assembly (200) to the position shown in FIG. 4A (associated with the open configuration of end effector (180) as shown in FIG. 5A). Therefore, if an operator releases jaw closure trigger (126), bias spring (155) will translate yoke assembly (200) to the position shown in FIG. 4A, thereby opening jaws (182, 184) of end effector (180).

As described above, and as shown between FIGS. 4B-4C and 5B-5C, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body (174) that is slidably coupled along proximal portion (144) of shaft assembly (140). Knife coupling body (174) is coupled with knife member (176) such that translation of knife coupling body (174) relative to proximal portion (144) of shaft assembly (140) translates knife member (176) relative to shaft assembly (140).

As best seen in FIGS. 4B-4C and 5B-5C, knife coupling body (174) is coupled a knife actuation assembly (168) such that as knife trigger (128) pivots toward body (122) and/or pistol grip (124), knife actuation assembly (168) drives knife coupling body (174) distally, thereby driving knife member (176) distally within knife pathway (192). Because knife coupling body (174) is coupled to knife member (176), knife member (176) translates distally within shaft assembly (140), articulation section (110), and within knife pathway (192) of end effector (180), as best shown between FIGS. 5B-5C. Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

Knife trigger (128) is biased to the positions seen in FIGS. 4A-4B (associated with the knife member (176) in the retracted position) by a bias arm (129). Bias arm (129) may include any suitable biasing mechanism as would be apparent to one having ordinary skill in the art in view of the teachings herein. For instance, bias arm (129) may include a torsion spring. Therefore, if an operator releases knife trigger (128), bias arm (129) returns knife trigger (128) to the position shown in FIGS. 4A-4B, thereby translating knife member (176) toward the retracted position.

With distal cutting edge (178) of knife member (176) actuated to the advance position (position shown in FIG. 5C), an operator may press activation button (130) to selectively activate electrode surfaces (194, 196) of jaws (182, 184) to weld/seal severed tissue that is captured between jaws (182, 184). It should be understood that the operator may also press activation button (130) to selectively activate electrode surfaces (194, 196) of jaws (182, 184) at any suitable time during exemplary use. Therefore, the operator may also press activation button (130) while knife member (176) is retracted as shown in FIGS. 3A-3B. Next, the operator may release jaw closure trigger (128) such that jaws (182, 184) pivot into the opened configuration, releasing tissue.

II. Examples of Lubricant Applicator Devices for Electrosurgical Instruments

As mentioned above, end effector (180) is configured to grasp, sever, and weld/seal tissue. In particular, jaw (184) may pivot relative to jaw (182) in order to grasp tissue, while knife member (176) is configured to actuate within jaws (182, 184) in order to sever tissue that is grasped between jaws (182, 184). Electrode surfaces (194, 196) may be activated while jaws (182, 184) grasp tissue in order to weld/seal tissue captured between jaws (182, 184). In some instances, it may be desirable to equip end effector (180) with an anti-stick material or lubricant to inhibit tissue from sticking to jaws (182, 184) during welding/sealing of the tissue. For example, it may be desirable for such lubricant to be applied to any one or more of the tissue-clamping surfaces (e.g., electrode surfaces (194, 196)) of jaws (182, 184) and/or to any one or more of the side surfaces (e.g., the laterally-outer edges and/or the distal tip) of jaws (182, 184). Such lubricant may include an anti-stick phospholipid solution, such as ELECTRO LUBE by Key Surgical of Eden Prairie, MN, and/or a medical dry film coating, such as TRIBOSYS MDF-A by Miller-Stephenson Chemical Company of Danbury, CT. It may be desirable for such lubricant to be distributed in a thin, uniform coating or layer to avoid interfering with the tissue welding/sealing performance of end effector (180). In some cases, it may be desirable to inhibit such lubricant from being applied to one or more regions of jaws (182, 184) that are proximal of the respective electrode surfaces (194, 196) and, more particularly, to inhibit such lubricant from reaching pivot couplings (198) and/or pin (164) to avoid interfering with the tissue grasping/clamping performance of end effector (180).

Because end effector (180) may be used to perform multiple welding/sealing operations during a single surgical procedure, it may be desirable to enable an operator to repeatedly and easily apply lubricant onto end effector jaws (182, 184) during that single surgical procedure. In some instances, it may also be desirable to enable the operator to thoroughly and easily clean each jaw (182, 184) prior to each application of lubricant thereto, and/or to enable the operator to easily and evenly distribute the applied lubricant over each jaw (182, 184) during or after each application of lubricant thereto. Each of the lubricant applicator devices described below provides one or more of these functionalities. Furthermore, any of the exemplary applicator devices described below may be suitably constructed for a single use or for multiple uses.
A. Exemplary Lubricant Applier Cartridge

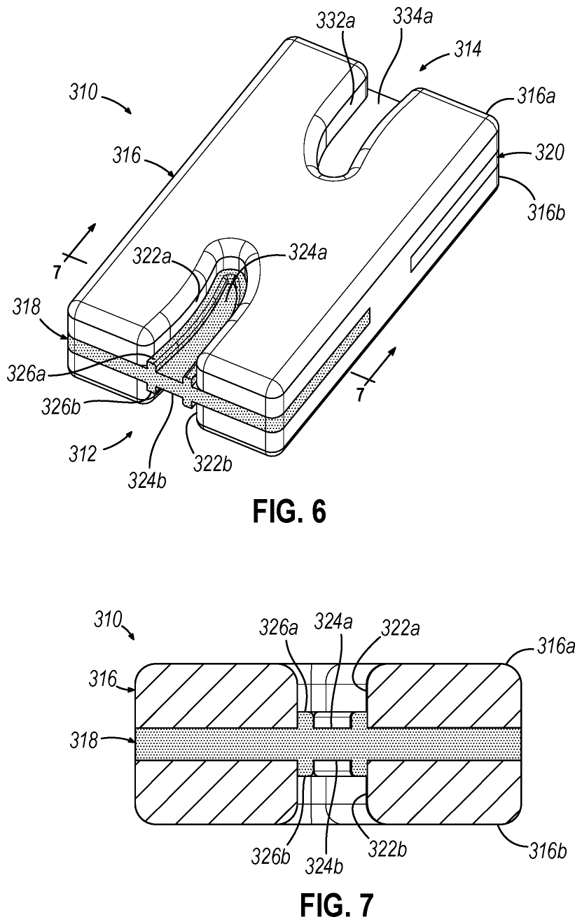

FIGS. 6-8D show an exemplary lubricant applicator in the form of a lubricant applier cartridge (310) that may be used to apply lubricant to end effector (180). As best seen in FIG. 6, cartridge (310) of this example comprises a first open end (312) and a second open end (314) opposite each other. Each open end (312, 314) is configured to receive end effector (180) as will be described in greater detail below. Cartridge (310) further includes a rigid housing (316) having upper and lower housing portions (316a, 316b) which each define an “H” shape to present open ends (312, 314). Housing (316) may be formed of a sterilizable material, such as plastic, to enable use of cartridge (310) in a sterile field. A lubricant application member in the form of a sponge (318) and a post-application excess lubricant removal member in the form of a cloth (320) are positioned at first and second open ends (312, 314), respectively, and are each interposed between first and second housing portions (316a, 316b).

Sponge (318) of the present example is configured to be loaded (e.g., saturated) with lubricant for applying the lubricant to jaws (182, 184) of end effector (180) on upper and lower sides of sponge (318). In this regard, the upper and lower sides of sponge (318) are at least partially exposed in upper and lower recesses (322a, 322b), respectively, that are formed between the prongs of the “H” configuration of upper and lower housing portions (316a, 316b) at first open end (312) and that collectively define a gap. More particularly, an upper application surface (324a) of sponge (318) is exposed in upper recess (322a) and a lower application surface (324b) of sponge (318) is exposed in lower recess (322b) for applying the lubricant to respective electrode surfaces (194, 196). In the example shown, sponge (318) includes upper and lower ridges (326a, 326b) extending along the peripheries of upper and lower recesses (322a, 322b), respectively, and defining the boundaries of upper and lower application surfaces (324a, 324b), respectively, for applying the lubricant to respective side surfaces of jaws (182, 184) while lubricant is being applied to electrode surfaces (194, 196) via application surfaces (324a, 324b). In this regard, recesses (322a, 322b), application surfaces (324a, 324b), and/or ridges (326a, 326b) may be sized and shaped (e.g., contoured) to accommodate the profiles of jaws (182, 184) such that electrode surfaces (194, 196) may contact the respective application surfaces (324a, 326b) simultaneously while the side surfaces of jaws (182, 184) contact the respective ridges (326a, 326b). For example, recesses (322a, 322b) may each have a shape complementary to that of the respective jaw (182, 184) and may have a size at least slightly larger than that of the respective jaw (182, 184), while ridges (326a, 326b) may each have a shape complementary to that of the respective jaw (182, 184) and may each have a size substantially equal to that of the respective jaw (182, 184) to promote engagement between each ridge (326a, 326b) and the side surfaces of the respective jaw (182, 184).

In addition, or alternatively, application surfaces (324a, 324b) may have a length selected to inhibit the regions of jaws (182, 184) that are proximal of the respective electrode surfaces (194, 196) (e.g., near pivot couplings (198) and/or pin (164)) from contacting any of application surfaces (324a, 324b) or ridges (326a, 326b) while electrode surfaces (194, 196) are engaged with the respective application surfaces (324a, 324b), and thereby inhibit application of lubricant to such proximal regions of jaws (182, 184). For example, application surfaces (324a, 324b) may each have a length substantially less than a distance between the distal tip of the corresponding jaw (182, 184) and such a proximal region of the corresponding jaw (182, 184). It will be appreciated that jaws (182, 184) may have various different sizes and shapes depending on the particular configuration of end effector (180), such that cartridge (310) may be configured for use with end effectors (180) of a particular configuration. For example, cartridge (310) may be configured for use with end effectors (180) having a relatively narrow curved configuration, a relatively wide curved configuration, and/or a straight configuration. In some versions, cartridge (310) may be configured for use with the ENSEAL® Tissue Sealing Device by Ethicon Endo-Surgery, Inc., of Cincinnati, Ohio.

Sponge (318) may be formed of any suitable material for absorbing, wicking, or otherwise drawing in the lubricant during initial loading (e.g., saturating) of sponge (318), subsequently holding or otherwise retaining the lubricant, and finally expelling at least a portion of the lubricant onto end effector (180). For example, sponge (318) may include a foam. In addition, or alternatively, sponge (318) may include a polymeric material such as polyurethane, polypropylene, or polyethylene. It will be appreciated that such materials may promote absorption of the lubricant at least in cases where the lubricant includes a hydrocarbon-based material such as an oily phospholipid. Sponge (318) may also be formed of a sterilizable material to enable use of cartridge (310) in the sterile field. Ridges (326a, 326b) may be formed on sponge (318) via any suitable manufacturing technique, such as cutting.

In some versions, sponge (318) may be pre-loaded (e.g., pre-saturated) with lubricant prior to packaging of cartridge (310), such that an operator may remove cartridge (310) from its packaging and immediately use cartridge (310) to apply lubricant to end effector (180) without needing to first load (e.g., saturate) sponge (318) with lubricant. In some other versions, cartridge (310) may be packaged with sponge (318) in an unloaded (e.g., unsaturated) state, such that the operator may remove cartridge (310) from its packaging and load sponge (318) with lubricant prior to using cartridge (310) to apply lubricant to end effector (180). Such loading of sponge (318) may be performed by depositing lubricant from a bottle or other container (not shown) onto sponge (318) and/or in any of the manners described herein. Sponge (318) may be configured to change color or shade when loaded with a predetermined amount of lubricant to provide a visual indication to the operator that sponge (318) has been sufficiently loaded.

Cloth (320) of the present example is configured to remove excess lubricant from jaws (182, 184) of end effector (180) on upper and lower sides of cloth (320) after lubricant has been applied thereto by sponge (318). In this regard, an upper removal surface (334a) of cloth (320) is exposed in an upper recess (332a) formed between the prongs of the “H” configuration of upper and lower housing portions (316a, 316b) at second open end (314) for removing the excess lubricant from a respective electrode surface (194, 196). While not shown, a lower removal surface of cloth (320) may similarly be exposed in a lower recess formed between the prongs of the “H” configuration of upper and lower housing portions (316a, 316b) for removing the excess lubricant from a respective electrode surface (194, 196). Such a lower recess and upper recess (332a) may collectively define a gap.

In the example shown, upper recess (332a) and removal surface (334a) and the lower recess and removal surface are each sized and shaped (e.g., contoured) to accommodate the profiles of jaws (182, 184) such that electrode surfaces (194, 196) may contact the respective upper removal surface (334a) or lower removal surface simultaneously. For example, upper recess (332a) and the lower recess may each have a shape complementary to that of the respective jaw (182, 184) and may have a size at least slightly larger than that of the respective jaw (182, 184).

Cloth (320) may be formed of any suitable material for absorbing, wicking or otherwise drawing at least a portion of the lubricant from end effector (180). In this regard, cartridge (310) may be packaged with cloth (320) in a substantially clean (e.g., lubricant-free) state. While cartridge (310) of the present example includes cloth (320), it will be appreciated that any other suitable type of excess lubricant removal member may be used. For example, another sponge (not shown) may be used in place of cloth (320) and may be formed of a material similar to that of sponge (318). While not shown, cartridge (310) may include a pre-application jaw cleaning member such as any of those described herein in addition to or in lieu of cloth (320).

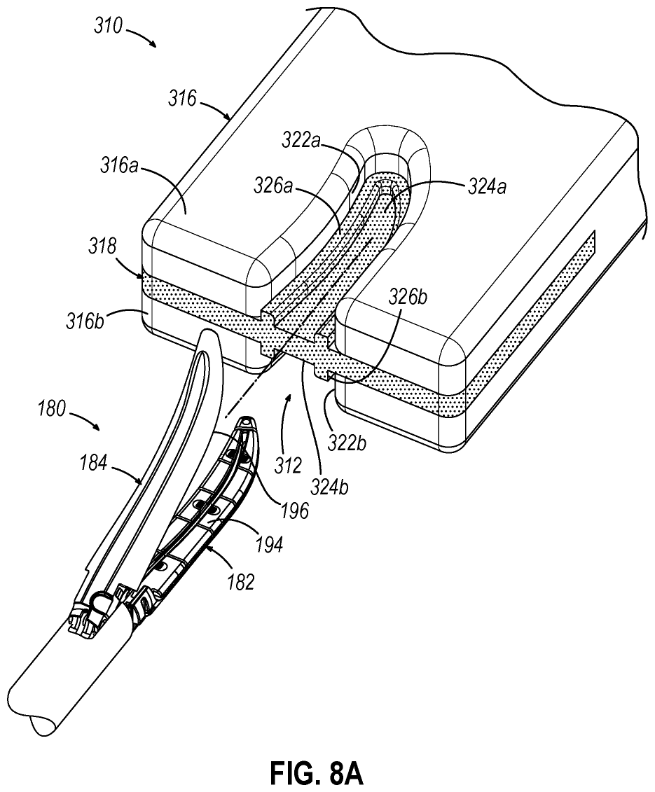

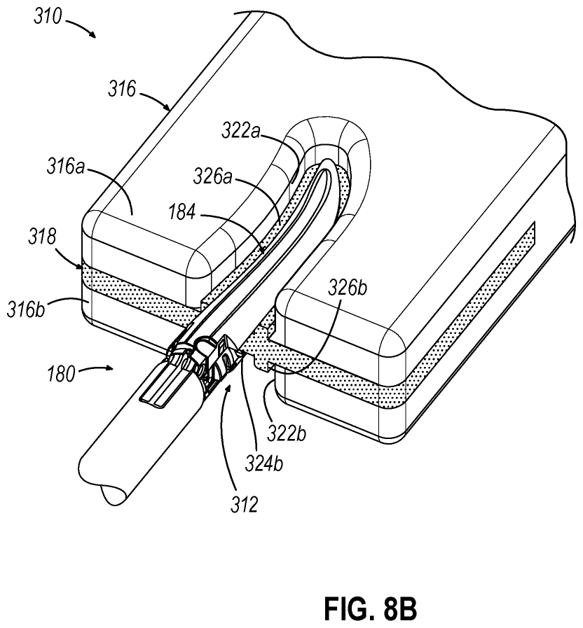

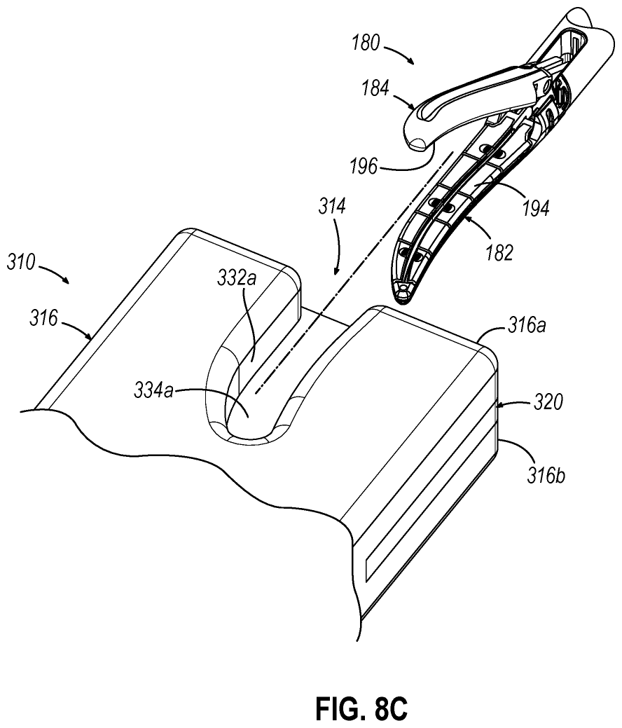

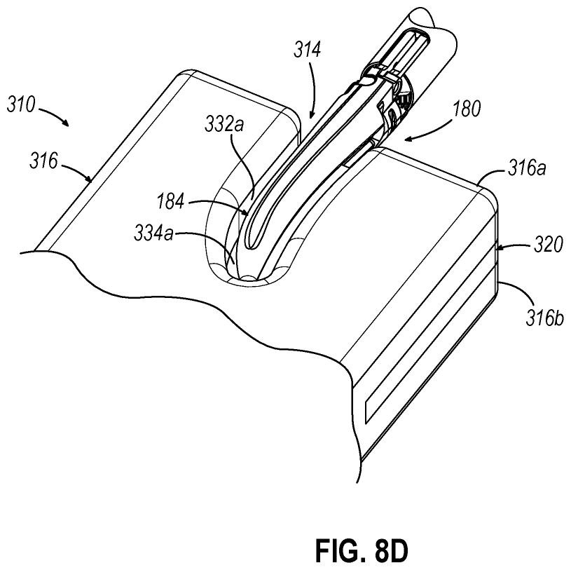

To use cartridge (310) to apply lubricant to end effector (180), the operator would first position cartridge (310) and end effector (180) such that end effector (180) is aligned with first open end (312) of cartridge (310) as shown in FIG. 8A. The operator would then advance end effector (180) distally, and/or advance cartridge (310) proximally, to position sponge (318) between electrode surfaces (194, 196) as shown in FIG. 8B. Closure trigger (126) of instrument (100) is then squeezed by the operator to close end effector jaws (182, 184) on sponge (318), thereby engaging electrode surfaces (194, 196) with the respective application surfaces (324a, 324b) while also engaging the side surfaces of jaws (182, 184) with the respective ridges (326a, 326b) such that at least some lubricant is expelled from sponge (318) onto each electrode surface (194, 196) and onto each side surface of jaws (182, 184). In some versions, sponge (318) may be at least slightly compressed between electrode surfaces (194, 196) to assist with expelling lubricant from sponge (318) onto end effector (180). In addition, or alternatively, the size and shape of recesses (322a, 322b), application surfaces (324a, 324b), and/or ridges (326a, 326b) relative to the profiles of end effector jaws (182, 184) may inhibit jaws (182, 184) from being withdrawn proximally relative to cartridge (310) while still closed on sponge (318) such that the operator may need to open jaws (182, 184) in order to disengage jaws (182, 184) from sponge (318). In any event, end effector jaws (182, 184) may be disengaged from sponge (318) while lubricant remains applied to electrode surfaces (194, 196) and the side surfaces of jaws (182, 184).

To use cartridge (310) to remove excess lubricant from end effector (180), the operator would position cartridge (310) and end effector (180) such that end effector (180) is aligned with second open end (314) of cartridge (310) as shown in FIG. 8C. The operator would then advance end effector (180) distally, and/or advance cartridge (310) proximally, to position cloth (320) between electrode surfaces (194, 196) as shown in FIG. 8D. Closure trigger (126) of instrument (100) is then squeezed by the operator to close end effector jaws (182, 184) on cloth (320), thereby engaging electrode surfaces (194, 196) with the respective removal surfaces (334a) such that excess lubricant is transferred onto cloth (320) from each electrode surface (194, 196) and/or from each side surface of jaws (182, 184). In some versions, the operator may advance end effector (180) proximally, and/or advance cartridge (310) distally, while end effector jaws (182, 184) are closed on cloth (320) and thereby wipe electrode surfaces (194, 196) along the respective removal surfaces (334a) to assist with transferring excess lubricant onto cloth (320) from end effector (180). In any event, end effector jaws (182, 184) may be disengaged from cloth (320) while a substantially uniform coating of lubricant remains applied to electrode surfaces (194, 196) and the side surfaces of jaws (182, 184). Due to the opposing arrangement of sponge (318) and cloth (320), an operator may be inhibited from accidentally positioning jaws (182, 184) over cloth (320) when intending to position jaws (182, 184) over sponge (318), or vice versa, or otherwise diverging from the proper sequence in which end effector (180) is to be moved between sponge (318) and cloth (320).

While not shown, cartridge (310) may include an ultraviolet light (UV) emitting device, such as a black light lamp or an ultraviolet light emitting diode (LED), secured to housing (316). In this regard, the lubricant applied to end effector (180) may be UV-sensitive such that the lubricant may glow in the presence of ultraviolet light. Thus, UV light may be emitted onto end effector (180) by the UV emitting device to enable the operator to visually discern whether a sufficient amount of lubricant has been uniformly applied to the desired surfaces of end effector (180), or whether further application or removal of lubricant may be desired.

In some versions, sponge (318) and/or cloth (320) may be removably interposed between first and second housing portions (316a, 316b), such as to permit replacement of a used sponge (318) and/or cloth (320) with a fresh sponge (318) and/or cloth (320) so that cartridge (310) may be suitable for multiple uses. In other versions, sponge (318) and/or cloth (320) may be permanently secured to housing (316). In such cases, cartridge (310) may be disposed of after completing the application of lubricant (L) to end effector (180).
B. Exemplary Lubricant Applier Cartridge for Multiple Jaw Configurations

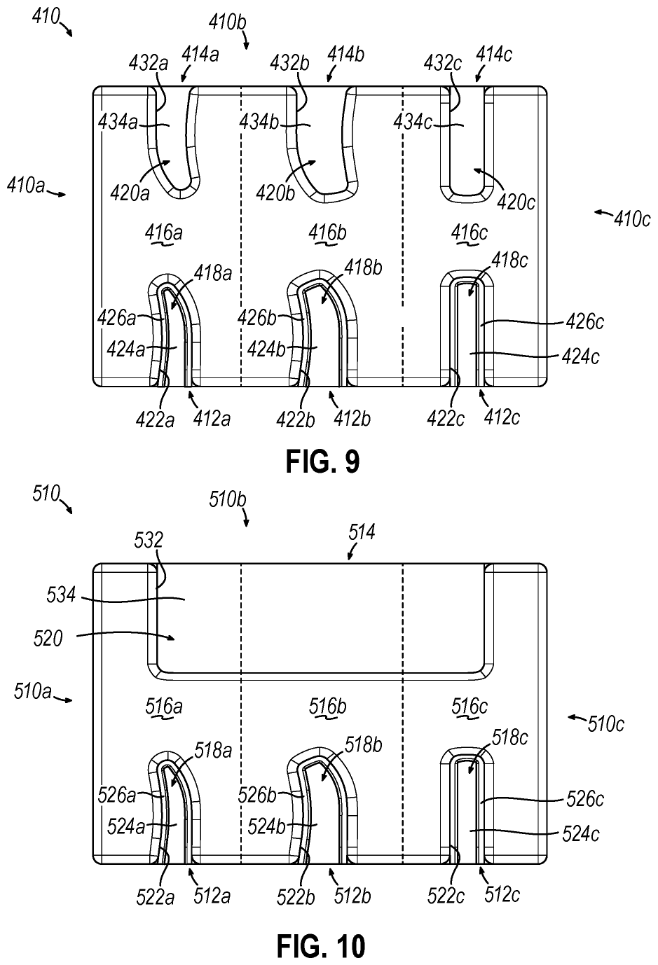

FIG. 9 shows another exemplary lubricant applicator in the form of a lubricant applier cartridge (410) that may be used to apply lubricant to end effector (180). Cartridge (410) is similar to cartridge (310) described above except as otherwise described below. In this regard, cartridge (410) of this example comprises a plurality of (e.g., three) integrated cartridge portions (410a, 410b, 410c) arranged side-by-side with each other, each including a respective first open end (412a, 412b, 412c) and a respective second open end (414a, 414b, 414c) opposite each other. Each open end (412a, 412b, 412c, 414a, 414b, 414c) is configured to receive a corresponding end effector (180) in a manner similar to that described above in connection with FIGS. 8A-8D. Each cartridge portion (410a, 410b, 410c) further includes at least one rigid housing portion (416a, 416b, 416c) which generally defines an “H” shape to present the respective open ends (412a, 412b, 412c, 414a, 414b, 414c).

A respective lubricant application member in the form of a sponge (418a, 418b, 418c) is positioned at each first open end (412a, 412b, 412c), and a post-application excess lubricant removal member in the form of a cloth (420a, 420b, 420c) is positioned at each second open end (414a, 414b, 414c). In some versions, sponges (418a, 418b, 418c) may be integrally formed together with each other as a unitary (e.g., monolithic) piece. In addition, or alternatively, cloths (420a, 420b, 420c) may be integrally formed together with each other as a unitary (e.g., monolithic ) piece.

An upper application surface (424a, 424b, 424c) of each sponge (418a, 418b, 418c) is exposed in a respective upper recess (422a, 422b, 422c) formed between the prongs of the “H” configuration of the respective housing portion (416a, 416b, 416c) and that define respective gaps. While not shown, a lower application surface of each sponge (418a, 418b, 418c) may similarly be exposed in a respective lower recess formed between the prongs of the “H” configuration of the respective housing portion (416a, 416b, 416c). In the example shown, each sponge (418a, 418b, 418c) includes a respective upper ridge (426a, 426b, 426c) extending along the periphery of the respective upper recesses (422a, 422b, 422c), and defining the boundary of the respective upper application surface (424a, 424b, 424c). While not shown, each sponge (418a, 418b, 418c) may similarly include a respective lower ridge extending along the periphery of the respective lower recess, and defining the boundary of the respective lower application surface. Recesses (422a, 422b, 422c), application surfaces (424a, 424b, 424c), and/or ridges (426a, 426b, 426c) of each cartridge portion (410a, 410b, 410c) may be sized and shaped (e.g., contoured) to accommodate jaw profiles of corresponding end effector configurations. For example, first ridge (426a) may have a complementary shape and a substantially equal size to a first jaw having a relatively narrow curved configuration, second ridge (426b) may have a complementary shape and a substantially equal size to a second jaw having a relatively wide curved configuration, and third ridge (426c) may have a complementary shape and a substantially equal size to a third jaw having a straight configuration, such that cartridge (410) may be configured for use with three distinct end effector configurations. In cases where sponges (418a, 418b, 418c) are integrally formed with each other as a unitary piece, it will be appreciated that lubricant saturation may be substantially uniform across application surfaces (424a, 424b, 424c) such that cartridge (410) may be used to apply lubricant to end effectors having the three distinct configurations (e.g., during a single surgical procedure) without necessarily requiring any of sponges (418a, 418b, 418c) to be reloaded (e.g., re-saturated) with lubricant.

An upper removal surface (434a, 434b, 434c) of each cloth (420a, 420b, 420c) is exposed in a respective upper recess (432a, 432b, 432c) formed between the prongs of the “H” configuration of the respective housing portion (416a, 416b, 416c) and that define respective gaps. While not shown, a lower removal surface of each cloth (420a, 420b, 420c) may similarly be exposed in a respective lower recess formed between the prongs of the “H” configuration of the respective housing portion (416a, 416b, 416c). Recesses (432a, 432b, 432c) and removal surfaces (434a, 434b, 434c) of each cartridge portion (410a, 410b, 410c) may be sized and shaped (e.g., contoured) to accommodate jaw profiles of corresponding end effector configurations. For example, first recess (432a) may have a complementary shape and a substantially equal size to the aforementioned first jaw, second recess (432b) may have a complementary shape and a substantially equal size to the aforementioned second jaw, and third recess (432c) may have a complementary shape and a substantially equal size to the aforementioned third jaw. While not shown, cartridge (410) may include at least one pre-application jaw cleaning member such as any of those described herein in addition to or in lieu of any one or more of cloths (420a, 420b, 420c).

While not shown, cartridge (410) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
C. Exemplary Lubricant Applier Cartridge for Multiple Jaw Configurations with Single Excess Lubricant Removal Member

FIG. 10 shows another exemplary lubricant applicator in the form of a lubricant applier cartridge (510) that may be used to apply lubricant to end effector (180). Cartridge (510) is similar to cartridge (410) described above except as otherwise described below. In this regard, cartridge (510) of this example comprises a plurality of (e.g., three) integrated cartridge portions (510a, 510b, 510c) arranged side-by-side with each other, each including a respective first open end (512a, 512b, 512c), and a second open end (514) opposite to first open ends (512a, 512b, 512c) and spanning each cartridge portion (510a, 510b, 510c). Each open end (512a, 512b, 512c, 514) is configured to receive a corresponding end effector (180) in a manner similar to that described above in connection with FIGS. 8A-8D. Each cartridge portion (510a, 510b, 510c) further includes at least one rigid housing portion (516a, 516b, 516c). A respective lubricant application member in the form of a sponge (518a, 518b, 518c) is positioned at each first open end (512a, 512b, 512c), and a post-application excess lubricant removal member in the form of a cloth (520) is positioned at second open end (514).

An upper application surface (524a, 524b, 524c) of each sponge (518a, 518b, 518c) is exposed in a respective upper recess (522a, 522b, 522c), and each sponge (518a, 518b, 518c) includes a respective upper ridge (526a, 526b, 526c) extending along the periphery of the respective upper recesses (522a, 522b, 522c), and defining the boundary of the respective upper application surface (524a, 524b, 524c). Recesses (522a, 522b, 522c), application surfaces (524a, 524b, 524c), and/or ridges (526a, 526b, 526c) of each cartridge portion (510a, 510b, 510c) may be sized and shaped (e.g., contoured) to accommodate jaw profiles of corresponding end effector configurations. For example, first ridge (526a) may have a complementary shape and a substantially equal size to a first jaw having a relatively narrow curved configuration, second ridge (526b) may have a complementary shape and a substantially equal size to a second jaw having a relatively wide curved configuration, and third ridge (526c) may have a complementary shape and a substantially equal size to a third jaw having a straight configuration, such that cartridge (510) may be configured for use with three distinct end effector configurations.

An upper removal surface (534) of cloth (520) is exposed in an upper recess (532), and a lower removal surface (not shown) may likewise be exposed in a lower recess (not shown). Recess (532) and removal surface (534) are sized to accommodate jaw profiles of a variety of end effector configurations. For example, recess (532) may be sized to accommodate any one or more of the aforementioned first, second, and/or third jaws. While not shown, cartridge (510) may include a pre-application jaw cleaning member such as any of those described herein in addition to or in lieu of cloth (520).

While not shown, cartridge (510) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
D. Exemplary Lubricant Applier Cartridge with Bonded Lubricant Application and Excess Lubricant Removal Members

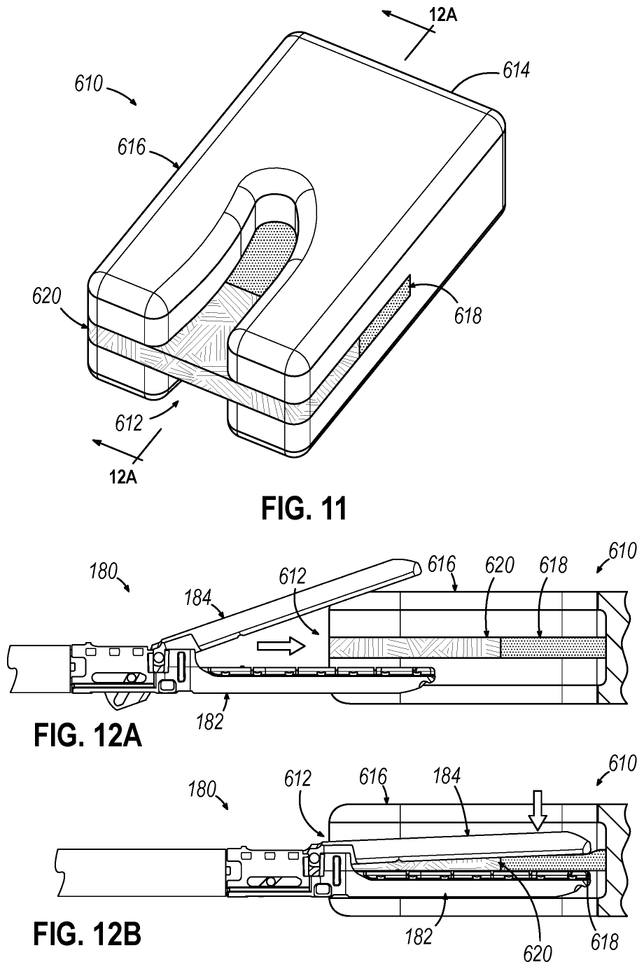

FIGS. 11-12B show another exemplary lubricant applicator in the form of a lubricant applier cartridge (610) that may be used to apply lubricant to end effector (180). Cartridge (610) is similar to cartridge (310) described above except as otherwise described below. In this regard, cartridge (610) of this example comprises an open end (612) and a closed end (614) opposite each other. Open end (612) is configured to receive end effector (180) in a manner similar to that described above in connection with FIGS. 8A-8D. Cartridge (610) further includes a rigid housing (616) which generally defines a “U” shape to present open end (612). A lubricant application member in the form of a first sponge (618) and a post-application excess lubricant removal member in the form of a second sponge (620) are secured to housing (616) at first open end (612).

First sponge (618) of the present example is configured to be loaded (e.g., saturated) with lubricant for applying the lubricant to jaws (182, 184) of end effector (180) on upper and lower sides of first sponge (618), and second sponge (620) of the present example is configured to remove excess lubricant from jaws (182, 184) of end effector (180) on upper and lower sides of second sponge (620) after lubricant has been applied thereto by first sponge (618). In this regard, first sponge (618) is distal of second sponge (620) (e.g., when end effector (180) is received by first open end (612)) and includes a relatively high density foam (e.g., with a relatively high stiffness), while second sponge (620) includes a relatively low density foam (e.g., with a relatively low stiffness) such that second sponge (620) may be configured to be compressed to a greater degree than first sponge (618) when end effector (180) is closed thereon. In the example shown, first sponge (618) and second sponge (620) are separately formed from each other as distinct pieces and subsequently bonded to each other. In some other versions, first sponge (618) and second sponge (620) may be integrally formed together with each other as a unitary (e.g., monolithic) piece. First sponge (618) and second sponge (620) may each have any suitable cross-sectional shape, such as rectangular or triangular, for example.

In some versions, first sponge (618) may be pre-loaded (e.g., pre-saturated) with lubricant prior to packaging of cartridge (610), such that an operator may remove cartridge (610) from its packaging and immediately use cartridge (610) to apply lubricant to end effector (180) without needing to first load (e.g., saturate) first sponge (618) with lubricant. In some other versions, cartridge (610) may be packaged with first sponge (618) in an unloaded (e.g., unsaturated) state, such that the operator may remove cartridge (610) from its packaging and load first sponge (618) with lubricant prior to using cartridge (610) to apply lubricant to end effector (180). In any event, cartridge (610) may be packaged with second sponge (620) in a substantially clean (e.g., lubricant-free) state.

To use cartridge (610) to apply lubricant to and remove excess lubricant from end effector (180), the operator would first position cartridge (610) and end effector (180) such that end effector (180) is aligned with first open end (612) of cartridge (610) as shown in FIG. 12A. The operator would then advance end effector (180) distally, and/or advance cartridge (610) proximally, to position first sponge (618) between electrode surfaces (194, 196) and second sponge (620) between proximal regions of jaws (182, 184) as shown in FIG. 12B. In some versions, second sponge (620) may be compressed between the proximal regions of jaws (182, 184) while jaws (182, 184) are open such that second sponge (620) crumples around the side surfaces of jaws (182, 184). Closure trigger (126) of instrument (100) is then squeezed by the operator to close end effector jaws (182, 184) on first and second sponges (618, 620), such that at least some lubricant is expelled from first sponge (618) onto each electrode surface (194, 196) and onto each side surface of jaws (182, 184). In some versions, first sponge (618) may be at least slightly compressed between electrode surfaces (194, 196) to assist with expelling lubricant from sponge (618) onto end effector (180). In any event, the operator may advance end effector (180) proximally, and/or advance cartridge (610) distally, while end effector jaws (182, 184) are closed on first and second sponges (618, 620) and thereby wipe electrode surfaces (194, 196) along second sponge (620) for facilitating removal of excess lubricant from end effector (180) onto second sponge (620). Due to the crumpling of second sponge (620) described above, second sponge (620) may unfurl during such relative movement between end effector (180) and cartridge (610) to assist with wiping excess lubricant onto second sponge (620) from end effector (180). In some versions, second sponge (620) may be compressed laterally inwardly and/or wrapped about one or both jaws (182, 184) to further increase contact between second sponge (620) and jaws (182, 184). In any event, end effector jaws (182, 184) may be disengaged from first and second sponges (618, 620) while a substantially uniform coating of lubricant remains applied to electrode surfaces (194, 196) and the side surfaces of jaws (182, 184).

While not shown, cartridge (610) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.

While first and second sponges (618, 620) have been described as being secured to housing (616) and incorporated into cartridge (610), it will be appreciated that housing (616) may be omitted in some versions such that first and second sponges (618, 620) may collectively define a standalone lubricant application and excess lubricant removal device.
E. Exemplary Lubricant Applier Cartridge with Layered Lubricant Application and Excess Lubricant Removal Members

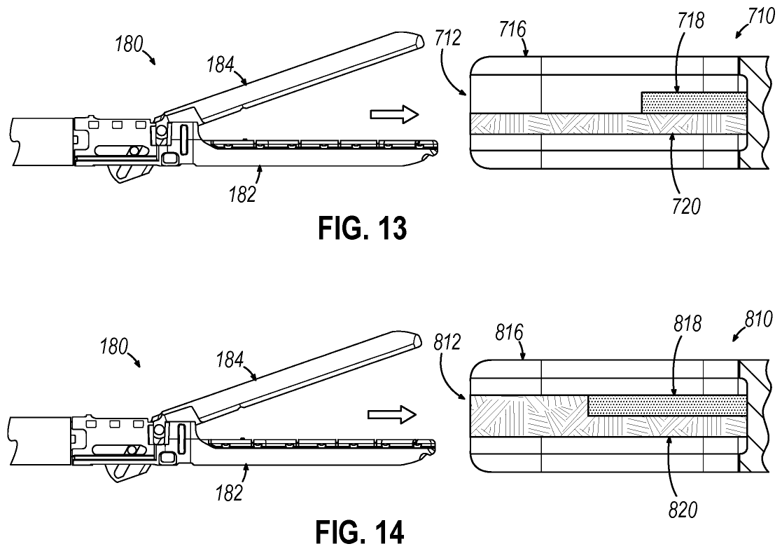

FIG. 13 shows another exemplary lubricant applicator in the form of a lubricant applier cartridge (710) that may be used to apply lubricant to end effector (180). Cartridge (710) is similar to cartridge (610) described above except as otherwise described below. In this regard, cartridge (710) of this example comprises a first open end (712). Open end (712) is configured to receive end effector (180) in a manner similar to that described above in connection with FIGS. 8A-8D. Cartridge (710) further includes a rigid housing (716) which generally defines a “U” shape to present open end (712). A first sponge (718) and a second sponge (720) are secured to housing (716) at first open end (712).

First sponge (718) of the present example is shorter than second sponge (720), is positioned over a distal portion of second sponge (720), and includes a relatively high density foam while second sponge (720) includes a relatively low density foam. In some versions, first sponge (718) and the underlying distal portion of second sponge (720) may be pre-loaded (e.g., pre-saturated) with lubricant prior to packaging of cartridge (710), such that an operator may remove cartridge (710) from its packaging and immediately use cartridge (710) to apply lubricant to end effector (180) without needing to first load (e.g., saturate) first sponge (718) and the underlying distal portion of second sponge (720) with lubricant. In some other versions, cartridge (710) may be packaged with first sponge (718) and the underlying distal portion of second sponge (720) in an unloaded (e.g., unsaturated) state, such that the operator may remove cartridge (710) from its packaging and load first sponge (718) and the underlying distal portion of second sponge (720) with lubricant prior to using cartridge (710) to apply lubricant to end effector (180). In any event, cartridge (710) may be packaged with a proximal portion of second sponge (720) in a substantially clean (e.g., lubricant-free) state, such that first sponge (718) and the underlying distal portion of second sponge (720) may collectively define the lubricant application member of cartridge (710) while the proximal portion of second sponge (720) may define the excess lubricant removal member of cartridge (710).

While not shown, cartridge (710) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
F. Exemplary Lubricant Applier Cartridge with Interlocked Lubricant Application and Excess Lubricant Removal Members

FIG. 14 shows another exemplary lubricant applicator in the form of a lubricant applier cartridge (810) that may be used to apply lubricant to end effector (180). Cartridge (810) is similar to cartridge (610) described above except as otherwise described below. In this regard, cartridge (810) of this example comprises an open end (812). Open end (812) is configured to receive end effector (180) in a manner similar to that described above in connection with FIGS. 8A-8D. Cartridge (810) further includes a rigid housing (816) which generally defines a “U” shape to present open end (812). A first sponge (818) and a second sponge (820) are secured to housing (816) at first open end (812).

Second sponge (820) of the present example is generally L-shaped and first sponge (818) of the present example is received against a shoulder defined by second sponge (820) over a distal portion of second sponge (820) and includes a relatively high density foam while second sponge (820) includes a relatively low density foam. In some versions, first sponge (818) and the underlying distal portion of second sponge (820) may be pre-loaded (e.g., pre-saturated) with lubricant prior to packaging of cartridge (810), such that an operator may remove cartridge (810) from its packaging and immediately use cartridge (810) to apply lubricant to end effector (180) without needing to first load (e.g., saturate) first sponge (818) and the underlying distal portion of second sponge (820) with lubricant. In some other versions, cartridge (810) may be packaged with first sponge (818) and the underlying distal portion of second sponge (820) in an unloaded (e.g., unsaturated) state, such that the operator may remove cartridge (810) from its packaging and load first sponge (818) and the underlying distal portion of second sponge (820) with lubricant prior to using cartridge (810) to apply lubricant to end effector (180). In any event, cartridge (810) may be packaged with a proximal portion of second sponge (820) in a substantially clean (e.g., lubricant-free) state, such that first sponge (818) and the underlying distal portion of second sponge (820) may collectively define the lubricant application member of cartridge (810) while the proximal portion of second sponge (820) may define the excess lubricant removal member of cartridge (810).

While not shown, cartridge (810) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
G. Exemplary Lubricant Applier Cartridge with Jaw Cleaning Member, Lubricant Application Member, and Excess Lubricant Removal Member

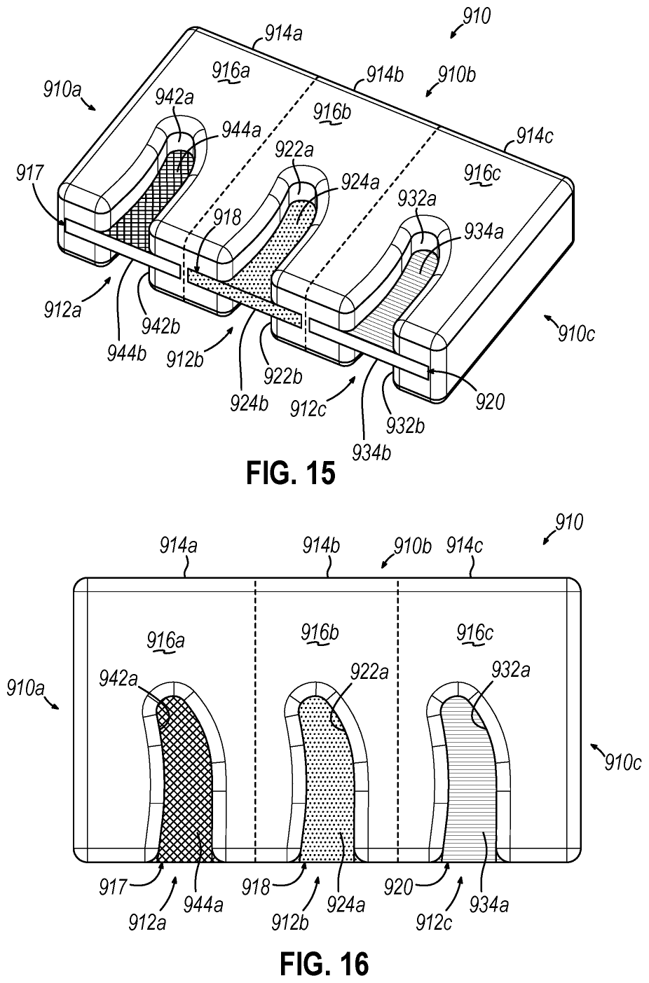

FIGS. 15-16 show another exemplary lubricant applicator in the form of a lubricant applier cartridge (910) that may be used to apply lubricant to end effector (180). Cartridge (910) is similar to cartridge (310) described above except as otherwise described below. In this regard, cartridge (910) of this example comprises a plurality of (e.g., three) integrated cartridge portions (910a, 910b, 910c) arranged side-by-side with each other, each including a respective open end (912a, 912b, 912c) and a respective closed end (914a, 914b, 914c) opposite each other. Each open end (912a, 912b, 912c) is configured to receive end effector (180) in a manner similar to that described above in connection with FIGS. 8A-8D. Each cartridge portion (910a, 910b, 910c) further includes at least one rigid housing portion (916a, 916b, 916c) which generally defines a “U” shape to present the respective open ends (912a, 912b, 912c).

A pre-application jaw cleaning member in the form of an abrasive (917) is secured to first housing portion (916a) at first open end (912a), a lubricant application member in the form of a sponge (918) is secured to second housing portion (916b) at second open end (912b), and a post-application excess lubricant removal member in the form of a cloth (920) is secured to third housing portion (916c) at third open end (912c). Upper and lower application surfaces (924a, 924b) of sponge (918) are exposed in upper and lower recesses (922a, 922b), respectively, formed between the prongs of the “U” configuration of second housing portion (916b), and upper and lower removal surfaces (934a, 934b) of cloth (920) are exposed in upper and lower recesses (932a, 932b), respectively, formed between the prongs of the “U” configuration of third housing portion (916c).

Abrasive (917) of the present example is configured to be loaded (e.g., saturated) with an aseptic cleaning solution (e.g., saline, isopropyl alcohol, etc.) for washing and/or scrubbing debris (e.g., tissue, eschar, etc.) from jaws (182, 184) of end effector (180) on upper and lower sides of abrasive (917) before lubricant is applied thereto by sponge (918). In this regard, the upper and lower sides of abrasive (917) are at least partially exposed in upper and lower recesses (942a, 942b), respectively, that are formed between the prongs of the “U” configuration of first housing portion (916a) at first open end (912a). More particularly, an upper cleaning surface (944a) of abrasive (917) is exposed in upper recess (942a) and a lower cleaning surface (944b) of abrasive (917) is exposed in lower recess (942b) for cleaning respective electrode surfaces (194, 196). In the example shown, recesses (942a, 942b) and cleaning surfaces (944a, 944b) are sized and shaped (e.g., contoured) to accommodate the profiles of jaws (182, 184) such that electrode surfaces (194, 196) may contact the respective cleaning surfaces (944a, 946b) simultaneously. For example, recesses (942a, 942b) may each have a shape complementary to that of the respective jaw (182, 184) and may have a size at least slightly larger than that of the respective jaw (182, 184).

Abrasive (917) may be formed of any suitable material for scrubbing, scraping or otherwise dislodging debris from end effector (180). For example, abrasive (917) may be formed of a substantially hard material and cleaning surfaces (944a, 944b) may each be substantially rough, coarse, or otherwise textured. In some versions, the texturing of cleaning surfaces (944a, 944b) may be configured to facilitate cleaning of knife pathway (192). In addition, or alternatively, abrasive (917) may be formed of any suitable material for absorbing, wicking, or otherwise drawing in the cleaning solution during initial loading (e.g., saturating) of abrasive (917), subsequently holding or otherwise retaining the cleaning solution, and finally expelling at least a portion of the cleaning solution onto end effector (180). For example, abrasive (917) may include another sponge (not shown) formed of a material (e.g., foam) having a higher stiffness than that of sponge (918). While cartridge (910) of the present example includes abrasive (917), it will be appreciated that any other suitable type of jaw cleaning member may be used. For example, a brush defined by a plurality of stiff bristles (not shown) may be used in place of abrasive (917). In such cases, the bristles may be sufficiently small to facilitate cleaning of knife pathway (192). In some other versions, a gauze strip (not shown) may be used in place of abrasive (917).

In some versions, abrasive (917) may be pre-loaded (e.g., pre-saturated) with cleaning solution prior to packaging of cartridge (910), such that an operator may remove cartridge (910) from its packaging and immediately use cartridge (910) to remove debris from end effector (180) without needing to first load (e.g., saturate) abrasive (917) with cleaning solution. In some other versions, cartridge (910) may be packaged with abrasive (917) in an unloaded (e.g., unsaturated) state, such that the operator may remove cartridge (910) from its packaging and load abrasive (917) with cleaning solution prior to using cartridge (910) to remove debris from end effector (180). Such loading of abrasive (917) may be performed by depositing cleaning solution from a bottle or other container (not shown) onto abrasive (917) and/or in any of the manners described below. In any event, abrasive (917) and sponge (918) may be isolated from each other, such as via an internal partition (not shown) defined by and/or between first and second housing portions (916a, 916b), to inhibit cleaning solution and/or lubricant from traveling between abrasive (917) and sponge (918).

To use cartridge (910) to remove debris from, apply lubricant to, and remove excess lubricant from end effector (180), the operator would first position cartridge (910) and end effector (180) such that end effector (180) is aligned with first open end (912a) of cartridge (910). The operator would then advance end effector (180) distally, and/or advance cartridge (910) proximally, to position abrasive (917) between electrode surfaces (194, 196). Closure trigger (126) of instrument (100) is then squeezed by the operator to close end effector jaws (182, 184) on abrasive (917), thereby engaging electrode surfaces (194, 196) with the respective cleaning surfaces (944a, 944b) such that at least some cleaning solution is expelled from abrasive (917) onto each electrode surface (194, 196) and onto each side surface of jaws (182, 184). In some versions, abrasive (917) may be at least slightly compressed between electrode surfaces (194, 196) to assist with expelling cleaning solution from abrasive (917) onto end effector (180). In any event, the operator may advance end effector (180) proximally, and/or advance cartridge (910) distally, while end effector jaws (182, 184) are closed on abrasive (917) and thereby scrub electrode surfaces (194, 196) along the respective cleaning surfaces (944a, 944b) for dislodging debris from end effector (180). In addition, or alternatively, the operator may press activation button (130) to selectively activate electrode surfaces (194, 196) of jaws (182, 184) while closed on abrasive (917) to heat up any debris on end effector (180) to assist with dislodging such debris from end effector (180). End effector jaws (182, 184) may be disengaged from abrasive (917) following sufficient cleaning of electrode surfaces (194, 196) and the side surfaces of jaws (182, 184). Cartridge (910) may then be used to apply lubricant to and remove excess lubricant from end effector (180) in manners similar to those described above in connection with FIGS. 8A-8D. Due to the side-by-side arrangement of abrasive (917), sponge (918), and cloth (920), cartridge (910) may not need to be reoriented (e.g., turned around) relative to end effector (180) when moving end effector (180) between abrasive (917), sponge (918), and cloth (920).

While not shown, cartridge (910) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
H. Exemplary Lubricant Applier Cartridge with Lubricant and Cleaning Solution Injection Ports

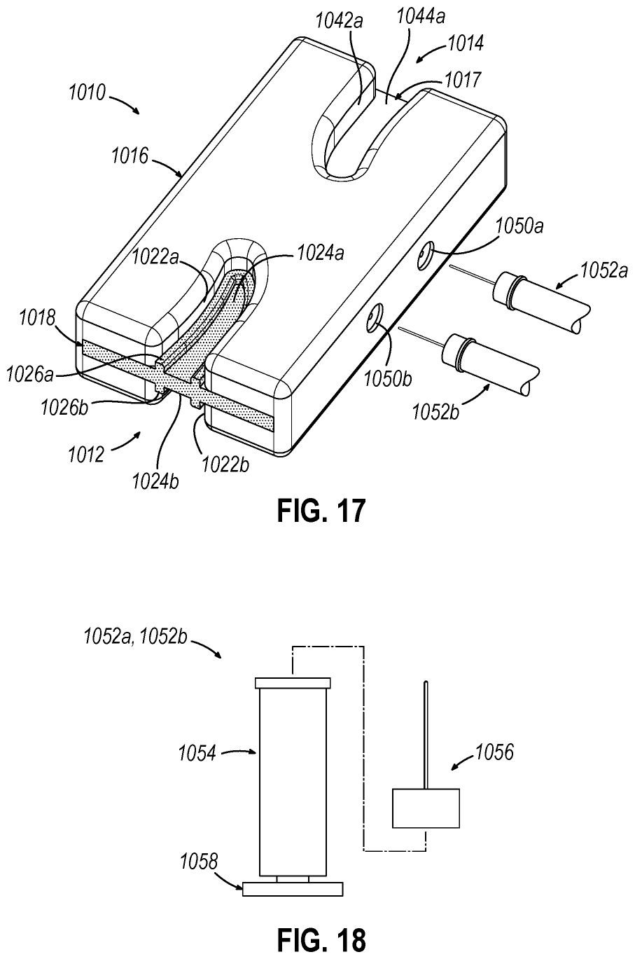

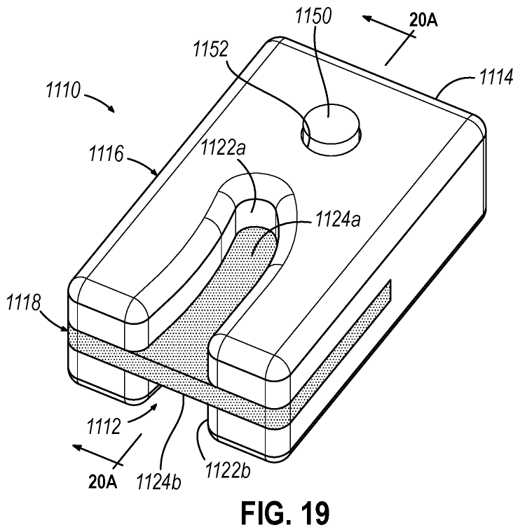

FIGS. 17-18 show another exemplary lubricant applicator in the form of a lubricant applier cartridge (1010) that may be used to apply lubricant to end effector (180). Cartridge (1010) is similar to cartridge (310) described above except as otherwise described below. In this regard, cartridge (1010) of this example comprises a first open end (1012) and a second open end (1014) opposite each other. Each open end (1012, 1014) is configured to receive end effector (180) in a manner similar to that described above in connection with FIGS. 8A-8D. Cartridge (1010) further includes a rigid housing (1016) which generally defines an “H” shape to present open ends (1012, 1014). A pre-application jaw cleaning member in the form of an abrasive (1017) and a lubricant application member in the form of a sponge (1018) are secured to housing (1016) at first and second open ends (1012, 1014), respectively. Upper and lower application surfaces (1024a, 1024b) of sponge (1018) are exposed in upper and lower recesses (1022a, 1022b), respectively, formed between the prongs of the “H” configuration of housing (1016), and upper and lower cleaning surfaces (upper cleaning surface (1044a) shown) of abrasive (1017) are exposed in upper and lower recesses (upper recess (1042a) shown), respectively, formed between the prongs of the “H” configuration of housing (1016). In the example shown, sponge (1018) includes upper and lower ridges (1026a, 1026b) extending along the peripheries of upper and lower recesses (1022a, 1022b), respectively, and defining the boundaries of upper and lower application surfaces (1024a, 1024b), respectively, for applying the lubricant to respective side surfaces of jaws (182, 184) while lubricant is being applied to electrode surfaces (194, 196) via application surfaces (1024a, 1024b). Abrasive (1017) and sponge (1018) are isolated from each other, such as via an internal partition (not shown) defined by housing (1016), to inhibit cleaning solution and/or lubricant from traveling between abrasive (1017) and sponge (1018).

Cartridge (1010) of the present example also includes first and second fluid injection ports (1050a, 1050b) extending laterally inwardly from a side surface of housing (1016) and in fluid communication with abrasive (1017) and sponge (1018), respectively, for facilitating loading (e.g., saturating) of abrasive (1017) and sponge (1018) with cleaning solution and lubricant, respectively. In this regard, fluid injection ports (1050a, 1050b) may facilitate loading of abrasive (1017) and sponge (1018), respectively, via corresponding syringe (1052a, 1052b).

Each syringe (1052a, 1052b) of the present example includes a barrel (1054) containing cleaning solution and lubricant, respectively, a needle (1056) detachably coupled to one end of the respective barrel (1054), and a plunger (1058) slidably fitted within the respective barrel (1054) and extending from the other end thereof for dispensing the cleaning solution or lubricant out of the respective barrel (1054) through the respective needle (1056). To that end, fluid injection ports (1050a, 1050b) may be sized and shaped to receive the needle (1056) of the corresponding syringe (1052a, 1052b). In the example shown, needles (1056) are detachable from the respective barrels (1054), such that each needle (1056) may be detached from the respective barrel (1054) after loading the respective abrasive (1017) or sponge (1018) and then disposed of without disposing of any cleaning solution or lubricant that may remain in the respective barrel (1054). In this manner, the remaining cleaning solution or lubricant may be preserved for a subsequent application using a fresh replacement needle (1056). In any event, it will be appreciated that abrasive (1017) and/or sponge (1018) may be initially loaded and/or reloaded for multiple uses of cartridge (1010) by inserting the needle (1056) of the corresponding syringe (1052a, 1052b) into the respective fluid injection port (1050a, 1050b) and dispensing the cleaning solution or lubricant.

While not shown, cartridge (1010) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.

In some versions, cartridge (1010) may be provided together with syringes (1052a, 1052b) to collectively define a lubricant applier kit.
I. Exemplary Lubricant Applier Cartridge with Lubricant Discharge Button

FIGS. 19-20B show another exemplary lubricant applicator in the form of a lubricant applier cartridge (1110) that may be used to apply lubricant to end effector (180). Cartridge (1110) is similar to cartridge (310) described above except as otherwise described below. In this regard, cartridge (1110) of this example comprises an open end (1112) and a closed end (1114) opposite each other. Open end (1112) is configured to receive end effector (180) in a manner similar to that described above in connection with FIGS. 8A-8D. Cartridge (1110) further includes a rigid housing (1116) which generally defines a “U” shape to present open end (1112). A lubricant application member in the form of a sponge (1118) is secured to housing (1116) at open end (1112). Upper and lower application surfaces (1124a, 1124b) of sponge (1118) are exposed in upper and lower recesses (1122a, 1122b), respectively, formed between the prongs of the “U” configuration of housing (1116).

Sponge (1118) of the present example further includes an internal channel (1125) (FIGS. 20A-20B) extending along a length of sponge (1118) for facilitating uniform loading (e.g., saturation) of sponge (1118) with lubricant. In this regard, a lubricant reservoir in the form of a rupturable pod (1127) is positioned at a distal end of channel (1125) (e.g., when end effector (180) is received by open end (1112)) and is configured to dispense lubricant (L) into channel (1125) such that the dispensed lubricant (L) may flow proximally along channel (1125) and may be soaked into sponge (1118) along substantially the entire length thereof.

Cartridge (1110) of the present example also includes a lubricant discharge member in the form of a button (1150) which is disposed in a bore (1152) extending through an upper side of housing (1116) over pod (1127). Button (1150) is configured to be depressed downwardly from an unactuated state (FIG. 20A) to an actuated state (FIG. 20B) for compressing pod (1127) against an underlying surface of housing (1116) to thereby rupture pod (1127) such that the lubricant (L) is dispensed from pod (1127) into channel (1125). In some versions, pod (1127) may be formed of foil, and may include perforations, serrations, or other pre-formed structural weaknesses (not shown) at or near a proximal end of pod (1127) such that pod (1127) may rupture at or near its proximal end to promote dispensing of the lubricant (L) proximally therefrom into channel (1125). In addition, or alternatively, button (1150) may be resiliently biased upwardly to the unactuated state via any suitable biasing member, such as a spring (not shown), such that button (1150) may resiliently return to the unactuated state from the actuated state. In some cases, a dampening member, such as a dashpot (not shown), may be configured to oppose such resilient biasing of button (1150) to slow the return of button (1150) to the unactuated state. In addition, or alternatively, button (1150) may be configured to be cyclically depressed to the actuated state and returned to the unactuated state for thoroughly dispensing lubricant (L) along channel (1125).

To use cartridge (1110) to apply lubricant to end effector (180), the operator would first remove cartridge (1110) from its packaging and load sponge (1118) with lubricant (L) by manually depressing button (1150) from the unactuated state shown in FIG. 20A to the actuated state shown in FIG. 20B such that lubricant (L) is directed through channel (1125) and soaked into sponge (1118). The operator may then position cartridge (1110) and end effector (180) such that end effector (180) is aligned with open end (1112) of cartridge (1110) and advance end effector (180) distally, and/or advance cartridge (1110) proximally, to position sponge (1118) between electrode surfaces (194, 196) as shown in FIG. 20B. Closure trigger (126) of instrument (100) is then squeezed by the operator to close end effector jaws (182, 184) on sponge (1118), thereby engaging electrode surfaces (194, 196) with the respective application surfaces (1124a, 1124b) such that at least some lubricant is expelled from sponge (1118) onto each electrode surface (194, 196) and onto each side surface of jaws (182, 184). In some versions, sponge (1118) may be at least slightly compressed between electrode surfaces (194, 196) to assist with expelling lubricant from sponge (1118) onto end effector (180). In any event, end effector jaws (182, 184) may be disengaged from sponge (1118) while lubricant remains applied to electrode surfaces (194, 196) and the side surfaces of jaws (182, 184).

While not shown, cartridge (1110) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
J. Exemplary Lubricant Applier Cartridge with Lubricant Discharge Button and One-Way Valve

FIG. 21 shows another exemplary lubricant applicator in the form of a lubricant applier cartridge (1210) that may be used to apply lubricant to end effector (180). Cartridge (1210) is similar to cartridge (1210) described above except as otherwise described below. In this regard, cartridge (1210) of this example comprises an open end (1212). Open end (1212) is configured to receive end effector (180) in a manner similar to that described above in connection with FIGS. 8A-8D. Cartridge (1210) further includes a rigid housing (1216) which generally defines a “U” shape to present open end (1212). A lubricant application member in the form of a sponge (1218) is secured to housing (1216) at open end (1212). Upper and lower application surfaces (1224a, 1224b) of sponge (1218) are exposed in upper and lower recesses (1222a, 1222b), respectively, formed between the prongs of the “U” configuration of housing (1216). Sponge (1218) includes an internal channel (1225) extending along a length of sponge (1218).

In the example shown, a lubricant reservoir in the form of a rupturable pod (1227) is housed within a lubricant discharge member in the form of a button (1250) which is disposed in a bore (1252) extending through an upper side of housing (1216) over a distal end of channel (1125) (e.g., when end effector (180) is received by open end (1212)). Button (1250) includes an upper button portion (1250a) and a lower button portion (1250b) defining a one-way valve (1253). Upper button portion (1250a) is configured to be depressed downwardly from the illustrated unactuated state to an actuated state (not shown) for compressing pod (1227) against an underlying surface of lower button portion (1250b) to thereby rupture pod (1227) such that the lubricant (L) is dispensed from pod (1227) into channel (1225) through valve (1253). In this regard, valve (1253) may be configured to selectively permit flow of lubricant (L) from pod (1227) toward channel (1225) (e.g., during depression of upper button portion (1250a)) and to restrict flow of lubricant (L) from channel (1225) toward pod (1227), and may thus inhibit inadvertent leakage of the lubricant (L). In addition, or alternatively, valve (1253) may prevent upper button portion (1250a) from returning to the unactuated state after at least a portion of the lubricant (L) has been dispensed into channel (1225). In such cases, the height of upper button portion (1250a) relative to housing (1216) may provide a visual indication of the amount of lubricant (L) that has been dispensed into channel (1225) and/or of the amount of lubricant (L) remaining in pod (1227). In some versions, upper button portion (1250a) may engage a ratchet (not shown) to provide an audible and/or tactile indication of the amount of lubricant (L) that has been dispensed into channel (1225) and/or of the amount of lubricant (L) remaining in pod (1227).

In some versions, pod (1227) may be removably housed within button (1250). For example, upper button portion (1250a) may be detachably coupled to lower button portion (1250b), such that upper button portion (1250a) may be selectively detached from lower button portion (1250b) for replacing a spent pod (1227) with a fresh pod (1227). While cartridge (1210) of the present example includes button (1250), it will be appreciated that any other suitable type of lubricant discharge member may be used. For example, a single-piece flexible membrane may be used in place of button (1250) and may include an internal chamber for containing a supply of the lubricant (L).

While not shown, cartridge (1210) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
K. Exemplary Lubricant Applier Cartridge with Lubricant Discharge Pump Bulb and One-Way Valves

FIGS. 22-23E show another exemplary lubricant applicator in the form of a lubricant applier cartridge (1310) that may be used to apply lubricant to end effector (180). Cartridge (1310) is similar to cartridge (310) described above except as otherwise described below. In this regard, cartridge (1310) of this example comprises a pair of integrated cartridge portions (1310a, 1310b) arranged side-by-side with each other, each including a respective open end (1312a, 1312b) and a respective closed end (1314a, 1314b) opposite each other. Each open end (1312a, 1312b) is configured to receive end effector (180) in a manner similar to that described above in connection with FIGS. 8A-8D. Each cartridge portion (1310a, 1310b) further includes at least one rigid housing portion (1316a, 1316b) which generally defines a “U” shape to present the respective open ends (1312a, 1312b).

A lubricant application member in the form of a sponge (1318) is secured to first housing portion (1316a) at first open end (1312a), and a post-application excess lubricant removal member in the form of a cloth (1320) is secured to second housing portion (1316b) at second open end (1312b). Upper and lower application surfaces (upper application surface (1324a) shown) of sponge (1318) are exposed in upper and lower recesses (upper recess (1322a) shown), respectively, formed between the prongs of the “U” configuration of first housing portion (1316a), and upper and lower removal surfaces (upper removal surface (1334a) shown) of cloth (1320) are exposed in upper and lower recesses (upper recess (1332a) shown), respectively, formed between the prongs of the “U” configuration of second housing portion (1316b). While not shown, cartridge (1310) may include a pre-application jaw cleaning member such as any of those described herein in addition to or in lieu of cloth (1320).

In the example shown, a plurality of internal channels (1325) extend through first housing portion (1316a) to various locations along a length of sponge (1318) for facilitating uniform loading (e.g., saturation) of sponge (1318) with lubricant. In this regard, a lubricant reservoir in the form of an internal compartment (1327) is defined within first housing portion (1316a) near first closed end (1314a) and is configured to dispense lubricant (L) into channels (1325) such that the dispensed lubricant (L) may flow proximally along channels (1325) and may be soaked into sponge (1318) along substantially the entire length thereof.

Cartridge (1310) of the present example also includes a lubricant discharge member in the form of a pump bulb (1350) which is disposed in a bore (1352) extending through an upper side of first housing portion (1316a) between compartment (1327) and channels (1325). Pump bulb (1350) is formed of a flexible membrane defining an internal chamber (1351) (FIGS. 23A-23E) and is equipped with a first one-way valve (1353a) positioned between chamber (1351) and compartment (1327) and a second one-way valve (1353b) positioned between chamber (1351) and channels (1325). Pump bulb (1350) is configured to be depressed downwardly from an unactuated state (FIGS. 23A, 23C, and 23E) to an actuated state (FIGS. 23B and 23D) for pumping the lubricant (L) from compartment (1327) into channels (1325). More particularly, pump bulb (1350) is configured to selectively fill chamber (1351) with lubricant (L) from compartment (1327) and to subsequently dispense such lubricant (L) into channels (1325). In this regard, valves (1353a, 1353b) may each be configured to selectively permit flow of lubricant (L) from compartment (1327) toward channels (1325) and to restrict flow of lubricant (L) from channels (1325) toward compartment (1327). In some versions, pump bulb (1350) may be resiliently biased upwardly to the unactuated state, such as by forming pump bulb (1350) of a resilient membrane, such that pump bulb (1350) may resiliently return to the unactuated state from the actuated state.

Pump bulb (1350) may be configured to be cyclically depressed to the actuated state and returned to the unactuated state to facilitate controlled (e.g., metered) pumping of lubricant (L) along channels (1325). For example, chamber (1351) of pump bulb (1350) may be configured to hold a predetermined amount of lubricant (L) corresponding to a volume of chamber (1351), such that each cycle may dispense the predetermined amount of lubricant (L) into channels (1325).

To use cartridge (1310) to apply lubricant to end effector (180), the operator would first remove cartridge (1310) from its packaging and load sponge (1318) with lubricant (L). In this regard, pump bulb (1350) may initially be in the unactuated state with each valve (1353a, 1353b) closed such that compartment (1327) is fluidly isolated from each of chamber (1351) and channels (1325), as shown in FIG. 23A. The operator may begin by manually depressing pump bulb (1350) from the unactuated state shown in FIG. 23A to the actuated state while each valve (1353a, 1353b) remains closed to thereby create a vacuum within chamber (1351), as shown in FIG. 23B. The operator may then release pump bulb (1350) to allow pump bulb (1350) to resiliently return to the unactuated state, such that the vacuum within chamber (1351) pulls a portion of the lubricant (L) from compartment (1327) into chamber (1351) through first valve (1353a) while second valve (1353b) remains closed, as shown in FIG. 23C. First valve (1353a) may close after chamber (1351) has been substantially filled with such lubricant (L).

Next, the operator may again manually depress pump bulb (1350) to the actuated state to push the portion of the lubricant (L) within chamber (1351) into channels (1325) through second valve (1353b) while first valve (1353a) remains closed, as shown in FIG. 23D, such that lubricant (L) is directed through channels (1325) and soaked into sponge (1318). Second valve (1353b) may close after chamber (1351) has been substantially emptied of such lubricant (L) to allow another vacuum to be created within chamber (1351). The operator may subsequently again release pump bulb (1350) to allow pump bulb (1350) to resiliently return to the unactuated state, such that the vacuum within chamber (1351) pulls another portion of the lubricant (L) from compartment (1327) into chamber (1351) through first valve (1353a) while second valve (1353b) remains closed, as shown in FIG. 23E. The operator may cyclically depress and release pump bulb (1350) until compartment (1327) has been substantially emptied of lubricant (L) or until a desired amount of the lubricant (L) has been directed through channels (1325) and soaked into sponge (1318). In this regard, the operator may perform a number of cycles which correlates to the desired amount of the lubricant (L). The operator may then proceed with positioning sponge (1318) between electrode surfaces (194, 196), closing end effector jaws (182, 184) on sponge (1318), and applying the lubricant (L) to end effector (180) in a manner similar to that described above in connection with FIGS. 8A-8D.

In some versions, pump bulb (1350) may be incorporated into a pre-application jaw cleaning member such as any of those described herein. For example, pump bulb (1350) may be encased within a compressible abrasive (not shown) or otherwise positioned relative to the abrasive such that pump bulb (1350) may be automatically depressed during compression of the abrasive between electrode surfaces (194, 196) or otherwise during cleaning of jaws (182, 184) prior to application of lubricant (L).

While not shown, cartridge (1310) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
L. Exemplary Lubricant Applier Cartridge with Lubricant Discharge Paddle

FIG. 24 shows another exemplary lubricant applicator in the form of a lubricant applier cartridge (1410) that may be used to apply lubricant to end effector (180). Cartridge (1410) is similar to cartridge (310) described above except as otherwise described below. In this regard, cartridge (1410) of this example comprises a pair of integrated cartridge portions (1410a, 1410b) arranged side-by-side with each other, each including a respective open end (1412a, 1412b) and a respective closed end (1414a, 1414b) opposite each other. Each open end (1412a, 1412b) is configured to receive end effector (180) in a manner similar to that described above in connection with FIGS. 8A-8D. Each cartridge portion (1410a, 1410b) further includes at least one rigid housing portion (1416a, 1416b) which generally defines a “U” shape to present the respective open ends (1412a, 1412b).

A lubricant application member in the form of a sponge (1418) is secured to first housing portion (1416a) at first open end (1412a), and a post-application excess lubricant removal member in the form of a cloth (1420) is secured to second housing portion (1416b) at second open end (1412b). Upper and lower application surfaces (1424a, 1424b) of sponge (1418) are exposed in upper and lower recesses (1422a, 1422b), respectively, formed between the prongs of the “U” configuration of first housing portion (1416a), and upper and lower removal surfaces (1434a, 1434b) of cloth (1420) are exposed in upper and lower recesses (1432a, 1432b), respectively, formed between the prongs of the “U” configuration of second housing portion (1416b). While not shown, cartridge (1410) may include a pre-application jaw cleaning member such as any of those described herein in addition to or in lieu of cloth (1420).

In the example shown, at least one internal channel (1425) extends through first housing portion (1416a) to at least one location along a length of sponge (1418) for facilitating loading (e.g., saturation) of sponge (1418) with lubricant. In this regard, a lubricant reservoir in the form of an internal compartment (1427) is defined within first housing portion (1416a) near first closed end (1414a) and is configured to dispense lubricant (L) into channel (1425) such that the dispensed lubricant (L) may flow proximally along channel (1425) and may be soaked into sponge (1418). In some versions, the lubricant (L) may be contained within a squeeze tube (not shown) disposed within compartment (1427).

Cartridge (1410) of the present example also includes a lubricant discharge member in the form of a paddle (1450) which extends across compartment (1427) and which is equipped with first and second bars (1451a, 1451b) extending laterally outwardly therefrom. First bar (1451a) extends outwardly from a side of second housing portion (1416b) via an elongate slot (1452) to present a slidable switch (1453). Paddle (1450) is configured to be translated proximally (e.g., when end effector (180) is received by first open end (1412a)) via proximal sliding of switch (1453) for pushing the lubricant (L) from compartment (1427) into channel (1425). In some versions, a one-way valve (not shown) may be positioned between compartment (1427) and at least a portion of channel (1425) and may be configured to selectively permit flow of lubricant (L) from compartment (1427) toward channel (1425) (e.g., during proximal translation of paddle (1450)) and to restrict flow of lubricant (L) from channel (1425) toward compartment (1427).

Paddle (1450) may be configured to be incrementally translated proximally to facilitate controlled (e.g., metered) dispensing of lubricant (L) into channel (1425). For example, indicia such as arrowheads (1454) positioned at predetermined intervals along slot (1452) may provide a visual indication of the amount of lubricant (L) that has been dispensed into channel (1425) and/or of the amount of lubricant (L) remaining in compartment (1427). In some versions, switch (1453) may be equipped with one or more indents or detents (not shown) configured to engage one or more detents or indents (not shown) provided at predetermined intervals along slot (1452) (e.g., corresponding to the positions of arrowheads (1454)) to provide an audible and/or tactile indication of the amount of lubricant (L) that has been dispensed into channel (1425) and/or of the amount of lubricant (L) remaining in compartment (1427). For example, compartment (1427) may be configured to hold a variable amount of lubricant (L) corresponding to a variable volume of compartment (1427) as determined by the location of paddle (1450), such that proximal translation of paddle (1450) through each interval may dispense a predetermined amount of lubricant (L) into channel (1425). In addition, or alternatively, a portion of second bar (1451b) may be visible through an elongate window (1455) to provide a visual indication of the amount of lubricant (L) that has been dispensed into channel (1425) and/or of the amount of lubricant (L) remaining in compartment (1427). For example, indicia such as one or more gauge markers (1456) may be provided at opposing ends of window (1455) to provide a visual indication of whether the amount of lubricant (L) remaining in compartment (1427) is at a full state, at an empty state, or therebetween.

While not shown, cartridge (1410) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
M. Exemplary Lubricant Applier Cartridge with Removable Sponge

FIGS. 25A-25B show another exemplary lubricant applicator in the form of a lubricant applier cartridge (1510) that may be used to apply lubricant to end effector (180).

Cartridge (1510) is similar to cartridge (310) described above except as otherwise described below. In this regard, cartridge (1510) of this example comprises a pair of integrated cartridge portions (1510a, 1510b) arranged side-by-side with each other, each including a respective open end (1512a, 1512b) and a respective closed end (1514a, 1514b) opposite each other. Each open end (1512a, 1512b) is configured to receive end effector (180) in a manner similar to that described above in connection with FIGS. 8A-8D. Each cartridge portion (1510a, 1510b) further includes an upper rigid housing portion (1516a, 1516c) and a lower rigid housing portion (1516b, 1516d) pivotably coupled to each other via at least one hinge in the form of a living hinge (1519) at or near closed ends (1514a, 1514b) such that cartridge (1510) may be transitioned between a closed state (FIG. 25A) and an open state (FIG. 25B). First lower housing portion (1516b) defines a “U” shape to present first open end (1512a), and second upper and lower housing portions (1516c, 1516d) each define a “U” shape to collectively present second open end (1512b) when cartridge (1510) is in the closed state. A pre-application jaw cleaning member in the form of a brush defined by a plurality of bristles (1517) is positioned at first open end (1512a) with each bristle (1517) extending downwardly from first upper housing portion (1516a) toward first lower housing portion (1516b) (e.g., at least when cartridge (1510) is in the closed state), and a lubricant application member in the form of a sponge (1518) is positioned at second open end (1512b) and is interposed between second upper and lower housing portions (1516c, 1516d).

Sponge (1518) of the present example is removably interposed between second upper and lower housing portions (1516c, 1516d) to permit replacement of a used sponge (1518) with a fresh sponge (1518) so that cartridge (1510) may be suitable for multiple uses. In this regard, second upper and lower housing portions (1516c, 1516d) include upper and lower ledges (1550a, 1550b), respectively, at second open end (1512b) that are configured to collectively retain sponge (1518) when cartridge (1510) is in the closed state. Ledges (1550a, 1550b) may be sized and shaped to provide an interference fit with sponge (1518) when cartridge (1510) is in the closed state that may be sufficient to inhibit dislodgement of sponge (1518) in an upper or lower direction while permitting sponge (1518) to be withdrawn proximally (e.g., when end effector (180) is received by second open end (1512b)) upon application of a threshold pulling force thereto (e.g., via end effector jaws (182, 184) closed thereon) without tearing sponge (1518). In some versions, sponge (1518) may include a foam pad, such as that packaged with ELECTRO LUBE by Key Surgical of Eden Prairie, MN.

Bristles (1517) of the present example are configured to remove debris from a single jaw (182, 184) of end effector (180) on a lower side of bristles (1517) before lubricant is applied thereto by sponge (918). In this regard, first lower housing portion (1516b) includes a recess (1552) at first open end (1512a) that is configured to receive bristles (1517) and a single end effector jaw (182, 184) when cartridge (1510) is in the closed state. In some versions, at least some bristles (1517) may be sized and shaped to provide a press fit with recess (1552) when cartridge (1510) is in the closed state to inhibit inadvertent transitioning of cartridge (1510) from the closed state toward the open state while permitting cartridge (1510) to be transitioned toward the open state upon application of a threshold separating force thereto. In some other versions, cartridge (1510) may include any suitable locking mechanism such as a pair of corresponding snaps (not shown) for selectively maintaining cartridge (1510) in the closed state. While not shown, cartridge (1510) may include a post-application excess lubricant removal member such as any of those described herein in addition to or in lieu of bristles (1517).

To use cartridge (1510) to remove debris from and apply lubricant to end effector (180), the operator would first remove sponge (1518) from its packaging and load sponge (1518) with lubricant, such as by depositing lubricant from a bottle or other container (not shown) onto sponge (1518) and spreading such lubricant evenly across sponge (1518). The operator may then install sponge (1518) within either the upper or lower ledge (1550a, 1550b) while cartridge (1510) is in the open state shown in FIG. 25B. Next the operator may transition cartridge (1510) from the open state to the closed state shown in FIG. 25A to thereby capture sponge (1518) between ledges (1550a, 1550b). The operator may then individually insert each jaw (182, 184) in turn into recess (1552) and may advance end effector (180) proximally, and/or advance cartridge (1510) distally and thereby scrub electrode surfaces (194, 196) along bristles (1517) for dislodging debris from end effector (180). Cartridge (1510) may then be used to apply lubricant to end effector (180) in a manner similar to that described above in connection with FIGS. 8A-8D. In some versions, jaws (182, 184) may be withdrawn proximally relative to cartridge (1510) while still closed on sponge (1518) to thereby pull the used sponge (1518) out of cartridge (1510), such as for disposing the used sponge (1518). In such cases, cartridge (1510) may release sponge (1518) without tearing sponge (1518).

While not shown, cartridge (1510) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
N. Exemplary Lubricant Applier Cartridge with Clamshell Configuration

FIGS. 26A-26B show another exemplary lubricant applicator in the form of a lubricant applier cartridge (1610) that may be used to apply lubricant to end effector (180). Cartridge (1610) is similar to cartridge (310) described above except as otherwise described below. In this regard, cartridge (1610) of this example comprises a pair of integrated cartridge portions (1610a, 1610b) arranged side-by-side with each other, each including a respective open end (1612a, 1612b) and a respective closed end (not shown) opposite each other. Each open end (1612a, 1612b) is configured to receive end effector (180) in a manner similar to that described above in connection with FIGS. 8A-8D. Each cartridge portion (1610a, 1610b) further includes an upper rigid housing portion (1616a, 1616b) and a lower rigid housing portion (1616c, 1616d) pivotably coupled to each other via at least one hinge (not shown) at or near the closed ends such that cartridge (1610) may be transitioned between an open state (FIG. 26A) and a closed state (FIG. 26B). First upper and lower housing portions (1616a, 1616c) each define a “U” shape to collectively present first open end (1612a) when cartridge (1610) is in the closed state, and second upper and lower housing portions (1616b, 1616d) each define a “U” shape to collectively present second open end (1612b) when cartridge (1610) is in the closed state. A pair of lubricant application members in the form of upper and lower sponges (1618a, 1618b) are positioned at first open end (1612a) and are secured to first upper and lower housing portions (1616a, 1616c), respectively, and a pair of post-application excess lubricant removal members in the form of upper and lower sponges (1620a, 1620b) are positioned at second open end (1612b) and are secured to second upper and lower housing portions (1616b, 1616d), respectively.

Upper and lower lubricant application sponges (1618a, 1618b) of the present example are secured within first upper and lower recesses (1650a, 1650b) of first upper and lower housing portions (1616a, 1616c), respectively, such that lubricant application sponges (1618a, 1618b) confront each other when cartridge (1610) is in the closed state. In the example shown, lower lubricant application sponge (1618b) includes a slit (1652) sized and shaped to receive a single end effector jaw (182, 184). In this manner, lubricant application sponges (1618a, 1618b) may be configured to sandwich one end effector jaw (182, 184) therebetween while the other end effector jaw (182, 184) is received within slit (1652) when cartridge (1610) is in the closed state for applying the lubricant to each of electrode surfaces (194, 196) and side surfaces of jaws (182, 184). In some versions, lubricant application sponges (1618a, 1618b) may each be pre-loaded (e.g., pre-saturated_ with lubricant prior to packaging of cartridge (1610), such that an operator may remove cartridge (1610) from its packaging and immediately use cartridge (1610) to apply lubricant to end effector (180) without needing to first load (e.g., saturate) sponges (1618a, 1618b) with lubricant.

Upper and lower excess lubricant removal sponges (1620a, 1620b) of the present example are secured within second upper and lower recesses (1651a, 1651b) of second upper and lower housing portions (1616b, 1616d), respectively, such that excess lubricant removal sponges (1620a, 1620b) confront each other when cartridge (1610) is in the closed state. In the example shown, lower excess lubricant removal sponge (1620b) includes a slit (1653) sized and shaped to receive a single end effector jaw (182, 184). In this manner, excess lubricant removal sponges (1620a, 1620b) may be configured to sandwich one end effector jaw (182, 184) therebetween while the other end effector jaw (182, 184) is received within slit (1653) when cartridge (1610) is in the closed state for removing excess lubricant from each of electrode surfaces (194, 196) and side surfaces of jaws (182, 184). While not shown, cartridge (1610) may include a pair of pre-application jaw cleaning members such as any of those described herein in addition to or in lieu of excess lubricant removal sponges (1620a, 1620b).

To use cartridge (1610) to apply lubricant to and remove excess lubricant from end effector (180), the operator would first position cartridge (1610) and end effector (180) such that end effector (180) is aligned with first open end (1612a) of cartridge (1610) as shown in FIG. 26A. The operator would then advance end effector (180) distally, and/or advance cartridge (1610) proximally, to insert one end effector jaw, such as lower jaw (182), into slit (1652) of lower lubricant application sponge (1618b) and to position the other end effector jaw, such as upper jaw (184), over lower lubricant application sponge (1618b). Closure trigger (126) of instrument (100) is then squeezed by the operator to close end effector jaws (182, 184) on lower lubricant application sponge (1618b) and cartridge (1610) is transitioned to the closed state, as shown in FIG. 26B, thereby engaging each electrode surface (194, 196) and side surface of jaws (182, 184) with at least one of lubricant application sponges (1618a, 1618b). In some versions, jaws (182, 184) may be withdrawn proximally relative to cartridge (1610) while still closed on lower lubricant application sponge (1618b) and/or while cartridge (1610) remains in the closed state to thereby assist with spreading the lubricant evenly on jaws (182, 184). In any event, end effector jaws (182, 184) may be disengaged from lubricant application sponges (1618a, 1618b) while lubricant remains applied to electrode surfaces (194, 196) and the side surfaces of jaws (182, 184).

The operator may subsequently insert one end effector jaw (182, 184) into slit (1653) of lower excess lubricant removal sponge (1620b) with the other end effector jaw (182, 184) positioned over lower excess lubricant removal sponge (1620b), close end effector jaws (182, 184) on lower excess lubricant removal sponge (1620b), transition cartridge (1610) to the closed state, and withdraw jaws (182, 184) proximally relative to cartridge (1610) for removing any excess lubricant from each electrode surface (194, 196) and side surface of jaws (182, 184).

While not shown, cartridge (1610) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
O. Exemplary Lubricant Applier Cartridge with Plurality of Lubricant Reservoirs

FIGS. 27-30 show another exemplary lubricant applicator in the form of a lubricant applier cartridge (1710) that may be used to apply lubricant to end effector (180). Cartridge (1710) is similar to cartridge (310) described above except as otherwise described below. In this regard, cartridge (1710) of this example comprises a plurality of (e.g., three) integrated cartridge portions (1710a, 1710b, 1710c) arranged side-by-side with each other and configured to permit end effector (180) to be closed thereon in a manner similar to that described above in connection with FIGS. 8A-8D. First cartridge portion (1710a) includes a pre-application jaw cleaning member in the form of a gauze strip (1717), second cartridge portion (1710b) includes a lubricant application member in the form of a hollow first sponge (1718), and third cartridge portion (1710c) includes a post-application excess lubricant removal member in the form of a second sponge (1720).

Gauze strip (1717) of the present example is configured to be loaded (e.g., saturated) with an aseptic cleaning solution for washing debris from jaws (182, 184) of end effector (180) on upper and lower sides of gauze strip (1717)) before lubricant is applied thereto by sponge (1718). In some versions, gauze strip (1717) may be pre-loaded (e.g., pre-saturated) with cleaning solution prior to packaging of cartridge (1710), such that an operator may remove cartridge (1710) from its packaging and immediately use cartridge (1710) to remove debris from end effector (180) without needing to first load (e.g., saturate) gauze strip (1717) with cleaning solution. In some other versions, cartridge (1710) may be packaged with gauze strip (1717) in an unloaded (e.g., unsaturated) state, such that the operator may remove cartridge (1710) from its packaging and load gauze strip (1717) with cleaning solution prior to using cartridge (1710) to remove debris from end effector (180).

First sponge (1718) of the present example includes an internal chamber (1725) extending along a length of first sponge (1718) for housing at least one lubricant reservoir in the form of a plurality of (e.g., three) rupturable pods (1727). In this regard, each pod (1727) is configured to be compressed between end effector jaws (182, 184) when end effector (180) is closed on first sponge (1718) to thereby rupture pods (1727) such that the lubricant is dispensed from each pod (1727) into chamber (1725) and soaked into first sponge (1718) for application to end effector (180). In some versions, pods (1727) may contain different amounts of lubricant. For example, each pod (1727) may contain a predetermined amount of lubricant for application to a corresponding longitudinal portion of end effector (180), with the proximal pod (1727) containing a first amount of lubricant for application to a relatively proximal portion of end effector (180), the distal pod (1727) containing a second amount of lubricant for application to a relatively distal portion of end effector (180), and the intermediate pod (1727) containing a third amount of lubricant for application to a portion of end effector (180) therebetween. Such amounts may be selected to compensate for the forces exerted by jaws (182, 184) in the proximal-distal direction during closure of jaws (182, 184) on first sponge (1718), such that an even layer of lubricant is distributed onto end effector (180). In some versions, pods (1727) may be formed of foil, and may include pre-formed structural weaknesses such as serrations or perforations (1728) (FIG. 28) to promote dispensing of the lubricant therefrom into chamber (1725). In addition, or alternatively, a proximal end of first sponge (1718) may be configured to inhibit application of lubricant to the regions of jaws (182, 184) that are proximal of the respective electrode surfaces (194, 196) (e.g., near pivot couplings (198) and/or pin (164)). In some other versions, at least a portion of first sponge (1718) may be omitted such that the lubricant may be applied directly to end effector (180) by pods (1727). In the example shown, first and second sponges (1718, 1720) are integrally formed together as a unitary (e.g., monolithic) piece.

As shown in FIG. 28, pods (1727) may be coupled to each other via a film (1729) having a pull tab (1731) to collectively define a lubricant reload (1733). As shown in FIGS. 29-30, a supply of such reloads (1733) may be stored within a dispenser (1735) having a spring (1737) for biasing the supply of reloads (1733) downwardly toward an aperture (1739) of dispenser (1735) for selectively dispensing each reload (1733) therethrough. In this regard, the respective pull tab (1731) of each reload (1733) may assist the operator with gripping and pulling the reload (1733) through aperture (1739).

To use cartridge (1710) to remove debris from, apply lubricant to, and remove excess lubricant from end effector (180), the operator would first load a plurality of fresh pods (1727) (e.g., a reload (1733)) into chamber (1725) of first sponge (1718) if chamber (1725) does not already contain fresh pods (1727). The operator would then sequentially close end effector jaws (182, 184) on each of gauze strip (1717), first sponge (1718) (thereby rupturing pods (1727)), and second sponge (1720) in manners similar to those described above in connection with FIGS. 8A-8D and 15-16. Due to the side-by-side arrangement of gauze strip (1717), first sponge (1718), and second sponge (1720), cartridge (1710) may not need to be reoriented (e.g., turned around) relative to end effector (180) when moving end effector (180) between gauze strip (1717), first sponge (1718), and second sponge (1720). In some versions, cartridge (1710) may include numerical indicia (not shown) for visually indicating the proper sequence in which end effector (180) is to be moved between gauze strip (1717), first sponge (1718), and second sponge (1720).

While not shown, cartridge (1710) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
P. Exemplary Lubricant Applier Cartridge with Lubricant Discharge Pump Bulb for Pumping Lubricant from External Source

FIGS. 31-32 show another exemplary lubricant applicator in the form of a lubricant applier cartridge (1810) that may be used to apply lubricant to end effector (180). Cartridge (1810) is similar to cartridge (310) described above except as otherwise described below. In this regard, cartridge (1810) of this example comprises a pair of integrated cartridge portions (1810a, 1810b) arranged side-by-side with each other, each including a respective open end (1812a, 1812b) and a respective closed end (1814a, 1814b) opposite each other. Each open end (1812a, 1812b) is configured to receive end effector (180) in a manner similar to that described above in connection with FIGS. 8A-8D. Each cartridge portion (1810a, 1810b) further includes at least one rigid housing portion (1816a, 1816b) which generally defines a “U” shape to present the respective open ends (1812a, 1812b). A pre-application jaw cleaning member in the form of an abrasive (1817) is secured to first housing portion (1816a) at first open end (1812a), and a lubricant application member in the form of a sponge (1818) is secured to second housing portion (1816b) at second open end (1812b). While not shown, cartridge (1810) may include a post-application excess lubricant removal member such as any of those described herein in addition to or in lieu of abrasive (1817).

In the example shown, a plurality of internal channels (1825) extend through second housing portion (1816b) to various locations along a length of sponge (1818) for facilitating uniform loading (e.g., saturation) of sponge (1818) with lubricant. In this regard, a lubricant reservoir in the form of an external container such as a bottle (1827) is configured to be selectively fluidly coupled to internal channels (1825) via a tube (1829), and to dispense lubricant (L) into channels (1825) such that the dispensed lubricant (L) may flow proximally along channels (1825) and may be soaked into sponge (1818) along substantially the entire length thereof.

Cartridge (1810) of the present example also includes a lubricant discharge member in the form of a pump bulb (1850) which is disposed in a bore (1852) extending through an upper side of second housing portion (1816b) between tube (1829) and channels (1825). Pump bulb (1850) is formed of a flexible membrane positioned over an internal chamber (1851) and is equipped with a first one-way valve (not shown) positioned between chamber (1851) and tube (1829) and at least one second one-way valve (not shown) positioned between chamber (1851) and channels (1825). Pump bulb (1850) is configured to be depressed downwardly from the illustrated unactuated state to an actuated state (not shown) for pumping the lubricant (L) from bottle (1827) into channels (1825). More particularly, pump bulb (1850) is configured to selectively fill chamber (1851) with lubricant (L) from bottle (1827) and to subsequently dispense such lubricant (L) into channels (1825). In this regard, the valves may each be configured to selectively permit flow of lubricant (L) from bottle (1827) toward channels (1825) and to restrict flow of lubricant (L) from channels (1825) toward bottle (1827). In some versions, pump bulb (1850) may be resiliently biased upwardly to the unactuated state, such as by forming pump bulb (1850) of a resilient membrane, such that pump bulb (1850) may resiliently return to the unactuated state from the actuated state.

Pump bulb (1850) may be configured to be cyclically depressed to the actuated state and returned to the unactuated state to facilitate controlled (e.g., metered) pumping of lubricant (L) along channels (1825). For example, chamber (1851) below pump bulb (1850) may be configured to hold a predetermined amount of lubricant (L) corresponding to a volume of chamber (1851), such that each cycle may dispense the predetermined amount of lubricant (L) into channels (1825). In addition, or alternatively, a portion of at least one channel (1825) may be visible through a corresponding window (1855) to provide a visual indication of whether lubricant (L) has been dispensed into channel (1425) and thus whether lubricant (L) has been loaded onto sponge (1818).

In the example shown, each cartridge portion (1810a, 1810b) is configured to be selectively covered by a corresponding cap (1860a, 1860b) to provide sterile barriers protecting abrasive (1817) and sponge (1818), respectively, from an external environment. In some versions, one or both caps (1860a, 1860b) may be transparent to permit the operator to visually observe the respective abrasive (1817) or sponge (1818), such as to assess whether abrasive (1817) or sponge (1818) has been loaded with cleaning solution or lubricant, respectively. Second cap (1860b) of the present example includes a pair of windows (1861) configured to align with corresponding windows (1855) of cartridge (1810) to provide a visual indication of whether lubricant (L) has been dispensed into channel (1425) and thus whether lubricant (L) has been loaded onto sponge (1818) while second cartridge portion (1810b) is covered by second cap (1860b). In some versions, second cartridge portion (1810b) may remain covered by second cap (1860b) during loading of sponge (1818) with lubricant until cartridge (1810) is used to apply the lubricant to end effector (180), to inhibit sponge (1818) from drying out or becoming contaminated before use.

To use cartridge (1810) to remove debris from and apply lubricant to end effector (180), the operator would first load sponge (1818) with lubricant (L). In this regard, the operator may begin by positioning a free end of tube (1829) within bottle (1827). The operator may then manually depress pump bulb (1850) a first time from the unactuated state to the actuated state to thereby create a vacuum within chamber (1851) and release pump bulb (1850) to allow pump bulb (1850) to resiliently return to the unactuated state, such that the vacuum within chamber (1851) pulls a portion of the lubricant (L) from bottle (1827) into chamber (1851). The operator may subsequently manually depress pump bulb (1850) a second time to the actuated state to push the portion of the lubricant (L) within chamber (1851) into channels (1825), such that lubricant (L) is directed through channels (1825) and soaked into sponge (1818). The operator may confirm that sponge (1818) has been loaded with lubricant (L) by visually observing at least one channel (1825) through the corresponding window(s) (1855, 1861). The operator may then remove first cap (1860a) from first cartridge portion (1810a) and load abrasive (1817) with cleaning solution in cases where abrasive (1817) is not pre-loaded with cleaning solution, such as by submerging abrasive (1817) into a container of cleaning solution (not shown). The operator may then proceed with positioning abrasive (1817) between electrode surfaces (194, 196), closing end effector jaws (182, 184) on abrasive (1817), and removing debris from end effector (180) in a manner similar to that described above in connection with FIGS. 15-16. Next, the operator may remove second cap (1860b) from second cartridge portion (1810b). The operator may then proceed with positioning sponge (1818) between electrode surfaces (194, 196), closing end effector jaws (182, 184) on sponge (1818), and applying the lubricant (L) to end effector (180) in a manner similar to that described above in connection with FIGS. 8A-8D. In some versions, cartridge (1810) may be disposed of after completing the application of lubricant (L) to end effector (180).

In the example shown, cartridge portions (1810a, 1810b) include respective numerical indicia (1862a, 1862b) for visually indicating the proper sequence in which end effector (180) is to be moved between abrasive (1817) and sponge (1818). Likewise, caps (1860a, 1860b) include respective numerical indicia (1863a, 1863b) for visually indicating the proper sequence in which caps (1860a, 1860b) are to be removed for facilitating the proper sequence in which end effector (180) is to be moved between abrasive (1817) and sponge (1818).

While not shown, cartridge (1810) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
Q. Exemplary Lubricant Applier Cartridge Multi-Pack

FIGS. 33-35 show another exemplary lubricant applicator in the form of a lubricant applier cartridge (1910) that may be used to apply lubricant to end effector (180). Cartridge (1910) is similar to cartridge (310) described above except as otherwise described below. In this regard, cartridge (1910) of this example comprises a first open end (1912) and a second open end (1914) opposite each other. Each open end (1912, 1914) is configured to receive end effector (180) in a manner similar to that described above in connection with FIGS. 8A-8D. Cartridge (1910) further includes a rigid housing (1916) which generally defines an “H” shape to present open ends (1912, 1914). A pre-application jaw cleaning member in the form of an abrasive (1917) and a lubricant application member in the form of a sponge (1918) are secured to housing (1916) at first and second open ends (1912, 1914), respectively. Upper and lower application surfaces (1924a, 1924b) of sponge (1918) are exposed in upper and lower recesses (1922a, 1922b), respectively, formed between the prongs of the “H” configuration of housing (1916), and upper and lower cleaning surfaces (upper cleaning surface (1944a) shown) of abrasive (1917) are exposed in upper and lower recesses (upper recess (1942a) shown), respectively, formed between the prongs of the “H” configuration of housing (1916). Abrasive (1917) and sponge (1918) are isolated from each other, such as via an internal partition (not shown) defined by housing (1916), to inhibit cleaning solution and/or lubricant from traveling between abrasive (1917) and sponge (1918). While not shown, cartridge (1910) may include a post-application excess lubricant removal member such as any of those described herein in addition to or in lieu of abrasive (1917).

Cartridge (1910) of the present example also includes a lubricant discharge member in the form of a pump bulb (1950) which is disposed in a bore (1952) extending through an upper side of housing (1916) for loading sponge (1918) with lubricant via internal channels (not shown) in a manner similar to that described above in connection with FIGS. 31-32. In other versions, sponge (1918) may be pre-loaded (e.g., pre-saturated) with lubricant prior to packaging of cartridge (1910), such that pump bulb (1950) may be omitted. In the example shown, removable caps (1960a, 1960b) are configured to provide sterile barriers protecting abrasive (1917) and sponge (1918), respectively, from an external environment.

Due to the opposing arrangement of abrasive (1917) and sponge (1918), an operator may be inhibited from accidentally positioning jaws (182, 184) over sponge (1918) when intending to position jaws (182, 184) over abrasive (1917), or vice versa, or otherwise diverging from the proper sequence in which end effector (180) is to be moved between abrasive (1917) and sponge (1918). In the example shown, cartridge (1910) includes numerical indicia (1962a, 1962b) for visually indicating the proper sequence in which end effector (180) is to be moved between abrasive (1917) and sponge (1918). Likewise, caps (1960a, 1960b) include respective numerical indicia (1963a, 1963b) for visually indicating the proper sequence in which caps (1960a, 1960b) are to be removed for facilitating the proper sequence in which end effector (180) is to be moved between abrasive (1917) and sponge (1918).

While not shown, cartridge (1910) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.

As shown in FIG. 33, a plurality of cartridges (1910) may be detachably coupled to each other to collectively define a cartridge multi-pack (1970). For example, each cartridge (1910) of multi-pack (1970) may be frangibly coupled to the adjacent cartridges (1910) of multi-pack (1970) such that each cartridge (1910) may be individually removed from multi-pack (1970) via application of a threshold breakaway force thereto, for facilitating use of the removed cartridge (1910) to remove debris from and apply lubricant to end effector (180).
R. Exemplary Lubricant Applier Cartridge with Lubricant and Cleaning Solution Wells

FIG. 36 shows another exemplary lubricant applicator in the form of a lubricant applier cartridge (2010) that may be used to apply lubricant to end effector (180). Cartridge (2010) is similar to cartridge (310) described above except as otherwise described below.

In this regard, cartridge (2010) of this example comprises a pair of integrated cartridge portions (2010a, 2010b) arranged side-by-side with each other, each including a plurality of wells (2012a, 2012b). Each well (2012a, 2012b) is configured to receive end effector (180) in a manner similar to that described above in connection with FIGS. 8A-8D. Each cartridge portion (2010a, 2010b) further includes at least one rigid housing portion (2016a, 2016b) which presents the respective wells (2012a, 2012b).

A plurality of pre-application jaw cleaning members in the form of abrasives (2017) are secured to first housing portion (2016a) within respective first wells (2012a) to thereby bifurcate each respective first well (2012a), and a plurality of lubricant application members in the form of sponges (2018) are secured to second housing portion (2016b) within respective second wells (2012b) to thereby bifurcate each respective second well (2012b). First and second cleaning surfaces (2044a, 2044b) of each abrasive (2017) are exposed in the respective first wells (2012a) for removing debris from respective electrode surfaces (194, 196) when the respective abrasive (2017) is sandwiched between end effector jaws (182, 184), and first and second application surfaces (2024a, 2024b) of each sponge (2018) are exposed in the respective second wells (2012b) for applying lubricant to respective electrode surfaces (194, 196) when the respective sponge (2018) is sandwiched between end effector jaws (182, 184).

In this regard, each first well (2012a) may be pre-loaded (e.g., pre-filled) with cleaning solution prior to packaging of cartridge (2010), at least some of which may be soaked into the respective abrasive (2017). Similarly, each second well (2012b) may be pre-loaded (e.g., pre-filled) with lubricant prior to packaging of cartridge (2010), at least some of which may be soaked into the respective sponge (2018).

In some versions, wells (2012a, 2012b) may be color-coded to provide a visual indication of whether each well (2012a, 2012b) contains cleaning solution or lubricant. In addition, or alternatively, wells (2012a, 2012b) may be color-coded to provide a visual indication of a particular jaw profile that each well (2012a, 2012b) is configured to accommodate. In this regard, the lefthand pair of first wells (2012a) and second wells (2012b) may be configured to accommodate a first jaw having a relatively narrow curved configuration, while the righthand pair of first wells (2012a) and second wells (2012b) may be configured to accommodate a second jaw having a relatively wide straight configuration, for example.

In the example shown, each cartridge portion (2010a, 2010b) is configured to be selectively covered by a peelable lid (2060) to provide a sterile barrier protecting each well (2012a, 2012b) and respective abrasive (2017) or sponge (2018) from an external environment. Peelable lid (2060) of the present example is configured to be peeled along a length of cartridge (2010) (e.g., left to right in the frame of reference of FIG. 36) to collectively uncover each well (2012a, 2012b).

While not shown, cartridge (2010) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive. For example, such a UV emitting device may be incorporated into a well similar to wells (2012a, 2012b).
S. Exemplary Lubricant Applier Cartridge with Lubricant and Cleaning Solution Wells and Multiple Peelable Lids

FIG. 37 shows another exemplary lubricant applicator in the form of a lubricant applier cartridge (2110) that may be used to apply lubricant to end effector (180). Cartridge (2110) is similar to cartridge (2010) described above except as otherwise described below. In this regard, cartridge (2110) of this example comprises a pair of integrated cartridge portions (2110a, 2110b) arranged side-by-side with each other, each including a plurality of wells (2112a, 2112b). Each well (2112a, 2112b) is configured to receive end effector (180) in a manner similar to that described above in connection with FIGS. 8A-8D. Each cartridge portion (2110a, 2110b) further includes at least one rigid housing portion (2116a, 2116b) which presents the respective wells (2112a, 2112b). A plurality of pre-application jaw cleaning members in the form of abrasives (2117) are secured to first housing portion (2116a) within respective first wells (2112a), and a plurality of lubricant application members in the form of sponges (2118) are secured to second housing portion (2116b) within respective second wells (2112b). First and second cleaning surfaces (2144a, 2144b) of each abrasive (2117) are exposed in the respective first wells (2112a), and first and second application surfaces (2124a, 2124b) of each sponge (2118) are exposed in the respective second wells (2112b).

In the example shown, each cartridge portion (2110a, 2110b) is configured to be selectively covered by a plurality of peelable lids (2160) to provide a sterile barrier protecting each well (2112a, 2112b) and respective abrasive (2117) or sponge (2118) from an external environment. Peelable lids (2160) of the present example each cover a respective first well (2112a) and adjacent second well (2112b), and are configured to be peeled along a width of cartridge (2110) (e.g., upwardly in the frame of reference of FIG. 37) to independently uncover each pair of adjacent wells (2112a, 2112b).

While not shown, cartridge (2110) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive. For example, such a UV emitting device may be incorporated into a well similar to wells (2112a, 2112b).
T. Exemplary Lubricant Applier Pouch

FIGS. 38A-38B show another exemplary lubricant applicator in the form of a lubricant applier pouch (2210) that may be used to apply lubricant to end effector (180). Pouch (2210) of this example comprises an openable end (2212) and a closed end (2214) opposite each other. When opened, openable end (2212) is configured to receive end effector (180) as will be described in greater detail below. Pouch (2210) further includes a laminated flexible housing (2216) which presents openable end (2212). A lubricant application member in the form of a sponge (2218) extends from closed end (2214) toward openable end (2212). A lubricant reservoir in the form of an internal compartment (2227) is defined within housing (2216) between closed end (2214) and an internal partition (2229). As shown, sponge (2218) bifurcates compartment (2227) and extends proximally therefrom through partition (2229). A cut-line (2231) extends parallel to partition (2229) and is positioned at least slightly proximally thereof to define a predetermined path along which a cutting tool (e.g., scissors) may cut housing (2216) to transition openable end (2212) from a closed state (FIG. 38A) to an opened state (FIG. 38B). In some versions, housing (2216) may include a tear notch or a resealable closure mechanism, such as a zipper, in place of cut-line (2231). In any event, first and second application surfaces (2224a, 2224b) of sponge (2218) are exposed outside of compartment (2227) when openable end (2212) is in the opened state for applying lubricant to respective electrode surfaces (194, 196).

In this regard, compartment (2227) may be pre-loaded (e.g., pre-filled) with lubricant, at least some of which may be soaked into sponge (2218), such that an operator may transition openable end (2212) to the open state and immediately use pouch (2210) to apply lubricant to end effector (180) without needing to first load sponge (2218) with lubricant.

Partition (2229) may be positioned relative to application surfaces (2224a, 2224b) to inhibit the regions of jaws (182, 184) that are proximal of the respective electrode surfaces (194, 196) (e.g., near pivot couplings (198) and/or pin (164)) from contacting any of application surfaces (2224a, 2224b) while electrode surfaces (194, 196) are engaged with the respective application surfaces (2224a, 2224b), and thereby inhibit application of lubricant to such proximal regions of jaws (182, 184). For example, application surfaces (2224a, 2224b) may each extend proximally relative to partition (2229) by a distance substantially less than a distance between the distal tip of the corresponding jaw (182, 184) and such a proximal region of the corresponding jaw (182, 184).

To use pouch (2210) to apply lubricant to end effector (180), the operator would first transition openable end (2212) from the closed state shown in FIG. 33A to the opened state shown in FIG. 33B. The operator would then position pouch (2210) and end effector (180) such that end effector (180) is aligned with openable end (2212) of pouch (2210), advance end effector (180) distally, and/or advance pouch (2210) proximally to position sponge (2218) between electrode surfaces (194, 196), and close end effector jaws (182, 184) on sponge (2218), thereby engaging electrode surfaces (194, 196) with the respective application surfaces (2224a, 2224b), as shown in FIG. 33B. Partition (2229) may inhibit jaws (182, 184) from being submerged in the lubricant (L) contained within compartment (2227). In any event, end effector jaws (182, 184) may be disengaged from sponge (2218) while lubricant remains applied to electrode surfaces (194, 196) and the side surfaces of jaws (182, 184).
U. Exemplary Blister Pack with Pre-Loaded Lubricant Applier Sponges

FIGS. 39-40B show a plurality of exemplary lubricant applicators in the form of sponges (2318) that may be used to apply lubricant to end effector (180). In the example shown, sponges (2318) are each pre-loaded (e.g., pre-saturated) with a predetermined amount of lubricant prior to being individually packaged (e.g., sealed) in respective pockets (2372) of a blister pack (2370), such that an operator may remove an individual sponge (2318) from the respective pocket (2372) of blister pack (2370) and immediately use the individual sponge (2318) to apply lubricant to end effector (180) without needing to first load (e.g., saturate) sponge (2318) with lubricant. Such a predetermined amount of lubricant may be selected for use with a particular end effector configuration. For example, each sponge (2318) may be pre-loaded with a predetermined amount of lubricant selected to uniformly apply a sufficient amount of lubricant to the desired surfaces of end effector (180) without needing to remove any excess lubricant therefrom.

To use an individual sponge (2318) to apply lubricant to end effector (180), the operator would first remove the individual sponge (2318) from the respective pocket (2372) of blister pack (2370) and align end effector (180) with the individual sponge (2318) as shown in FIG. 40A. The operator would then advance end effector (180) distally, and/or advance the individual sponge (2318) proximally, to position sponge (2318) between electrode surfaces (194, 196) and close end effector jaws (182, 184) on sponge (2318), thereby engaging electrode surfaces (194, 196) with respective application surfaces of sponge (2318), as shown in FIG. 40B. In some versions, sponge (2318) may be at least slightly compressed between electrode surfaces (194, 196) to assist with expelling lubricant from sponge (2318) onto end effector (180). In any event, end effector jaws (182, 184) may be disengaged from sponge (2318) while lubricant remains applied to electrode surfaces (194, 196) and the side surfaces of jaws (182, 184).
V. Exemplary Lubricant Applier Wedge

FIGS. 41-45 show another exemplary lubricant applicator in the form of a lubricant applier wedge (2410) that may be used to apply lubricant to end effector (180). Wedge (2410) of this example comprises a narrow end (2412) and a wide end (2414) opposite each other. Narrow end (2412) is configured to be received by end effector (180) as will be described in greater detail below. Wedge (2410) further includes a rigid manifold (2416) which defines a triangular shape to present narrow and wide ends (2412, 2414). Manifold (2416) may be formed of a sterilizable material, such as plastic, to enable use of wedge (2410) in a sterile field. As best shown in FIG. 42, manifold (2416) includes a main body portion (2416a), a pair of flanges (2416b) each extending both upwardly and downwardly from respective lateral sides of main body portion (2416a), and upper and lower central rails (2416c) extending upwardly and downwardly, respectively, from a laterally central region of main body portion (2416a), such that manifold (2416) has a “double E” cross-sectional shape. A plurality of lubricant application members in the form of lubricant application surfaces (2424) are defined on upper and lower sides of main body portion (2416a) and on laterally-inner sides of flanges (2416b) for applying lubricant to respective electrode surfaces (194, 196) and/or side surfaces of jaws (182, 184). A pre-application jaw cleaning member in the form of a substantially rough, coarse, or otherwise textured cleaning surface (2444) is defined on a laterally-outer side of at least one flange (2416b) for removing debris from any of electrode surfaces (194, 196) and/or side surfaces of jaws (182, 184). While not shown, wedge (2410) may include a post-application excess lubricant removal member such as any of those described herein in addition to or in lieu of cleaning surface (2444). For example, cleaning surface (2444) may be defined on the laterally-outer side of one flange (2416b) and an excess lubricant removal surface (not shown) may be defined on the laterally-outer side of the other flange (2416b).

In the example shown, a plurality of internal distribution channels (2425) extend through manifold (2416) to various locations along the lengths of each application surface (2424) for facilitating uniform application of lubricant to the desired surfaces of end effector (180). In this regard, a lubricant reservoir in the form of a squeeze tube (2427) is secured to manifold (2416) at wide end (2414) and is configured to dispense lubricant (L) into channels (2425) such that the dispensed lubricant (L) may flow proximally along channels (2425) and may be applied directly to the desired surfaces of end effector (180) along substantially the entire lengths thereof. Squeeze tube (2427) is formed of a flexible membrane defining an internal chamber (2451) (FIG. 44) and is configured to be depressed proximally from the illustrated unactuated state to an actuated state (not shown) for pushing the lubricant (L) from chamber (2451) into channels (2425). In some versions, a one-way valve (not shown) may be positioned between chamber (2451) and channels (2425) and may be configured to selectively permit flow of lubricant (L) from chamber (2451) toward channels (2425) (e.g., during proximal depression of squeeze tube (2427)) and to restrict flow of lubricant (L) from channels (2425) toward chamber (2451). In some versions, squeeze tube (2427) may be transparent to permit the operator to visually observe the contents of chamber (2451), such as to assess whether the lubricant (L) has been emptied therefrom.

Squeeze tube (2427) may be pre-loaded (e.g., pre-filled) with a predetermined amount of lubricant prior to packaging of wedge (2410), such that an operator may remove wedge (2410) from its packaging and immediately use wedge (2410) to apply lubricant to end effector (180) without needing to first load (e.g., fill) squeeze tube (2427) with lubricant. Such a predetermined amount of lubricant may be selected for use with a particular end effector configuration. For example, squeeze tube (2427) may be pre-loaded with a predetermined amount of lubricant selected to uniformly apply a sufficient amount of lubricant to the desired surfaces of end effector (180) without needing to remove any excess lubricant therefrom.

To use wedge (2410) to apply lubricant to end effector (180), the operator would first position wedge (2410) and end effector (180) such that end effector (180) is aligned with narrow end (2412) of wedge (2410) and would then advance end effector (180) distally, and/or advance wedge (2410) proximally, to engage narrow end (2412) of wedge (2410) with one or more tissue stops (not shown) of end effector (180), such that electrode surfaces (194, 196) and the side surfaces of jaws (182, 184) each confront respective application surfaces (2424) while jaws (182, 184) remain in an open state as shown in FIGS. 44-45. Squeeze tube (2427) is then depressed proximally such that lubricant (L) is directed through channels (2425) and expelled therefrom onto each electrode surface (194, 196) and onto each side surface of jaws (182, 184). The operator may visually discern that a sufficient amount of lubricant has been uniformly applied to the desired surfaces of end effector (180) by observing that the lubricant (L) has been emptied from chamber (2451). In some versions, central rails (2416c) are sized and shaped to engage upper and lower knife pathway portions (192a, 192b) of end effector (180) when narrow end (2412) of wedge (2410) is engaged with the tissue stop(s) of end effector (180) to promote proper alignment of wedge (2410) with end effector (180) and/or to provide a clearance gap between each application surface (2424) and the respective electrode surfaces (194, 196) and/or side surfaces of jaws (182, 184). Such clearance gaps may assist with distribution of lubricant from channels (2425) onto the respective electrode surfaces (194, 196) and/or side surfaces of jaws (182, 184). In any event, end effector jaws (182, 184) may be disengaged from manifold (2416) while lubricant remains applied to electrode surfaces (194, 196) and the side surfaces of jaws (182, 184).

While not shown, wedge (2410) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.
W. Exemplary Laparoscopic Lubricant Applier Assembly

FIGS. 46A-47B show another exemplary lubricant applicator in the form of a laparoscopic lubricant applier assembly (2510) that may be used to apply lubricant to end effector (180). Assembly (2510) of this example comprises a plunger (2512) and a capsule (2514) configured to be collectively inserted into an anatomical cavity (e.g., abdominal cavity, thoracic cavity, etc.) of a patient via a trocar (T). Capsule (2514) is configured to be received by end effector (180) in vivo as will be described in greater detail below. Capsule (2514) includes a rigid base (2516) which may be formed of silicone, for example. A pre-application jaw cleaning member in the form of a gauze strip (2517) and a lubricant application member in the form of a sponge (2518) are wrapped about respective portions of base (2516) such that a cleaning surface (2544) of gauze strip (2517) and an application surface (2524) of sponge (2518) each face outwardly away from base (2516). Base (2516) includes a proximal socket (2570) and a distal bore (2572) (FIGS. 47A-47B) configured to selectively receive corresponding portions of plunger (2512) as described in greater detail below.

In the example shown, a plurality of outlets (2575) (FIGS. 47A-47B) extend from bore (2572) through base (2516) to various locations along a length of sponge (2518) for facilitating uniform loading (e.g., saturation) of sponge (2518) with lubricant. In this regard, a lubricant reservoir in the form of a rupturable pod (2577) is positioned at a distal end of bore (2572) and is configured to dispense lubricant (L) into bore (2572) such that the dispensed lubricant (L) may flow outwardly through outlets (2575) and may be soaked into sponge (2518) along substantially the entire length thereof.

Plunger (2512) includes a proximal grip (2580), a distal rod (2582), and a plug (2584) positioned therebetween. Rod (2582) is configured to be inserted into bore (2572) of capsule (2514) and advanced distally therein from a proximal state (FIGS. 46A and 47A) to a distal state (FIGS. 46B and 47B) for compressing pod (2577) against a distal end of bore (2572) to thereby rupture pod (2577) such that the lubricant (L) is dispensed from pod (2577) into outlets (2575). As shown, plug (2584) is configured to engage socket (2570) when rod (2582) is in the distal state to inhibit further distal advancement of rod (2582) beyond the distal state. In some versions, plug (2584) is configured to be securely seated within socket (2570) (e.g., via a snap-fit) to also inhibit proximal advancement of rod (2582) from the distal state, such that plunger (2512) may be fixed against any additional movement relative to capsule (2514) to define an assembled state of assembly (2510). In such cases, the interaction between plug (2584) and socket (2570) may provide an audible and/or tactile indication that lubricant (L) has been dispensed into outlets (2575).

In some versions, gauze strip (2517) may be pre-loaded (e.g., pre-saturated) with cleaning solution prior to packaging of capsule (2514). In some other versions, a cleaning solution reservoir in the form of another rupturable pod (not shown) may be positioned at a proximal end of bore (2572) and may be configured to dispense cleaning solution into bore (2572) such that the dispensed cleaning solution may flow outwardly through additional outlets (not shown) and may be soaked into gauze strip (2517) along substantially the entire length thereof. For example, rod (2582) may rupture such another pod in a manner similar to that described above. In some other versions, the operator may load gauze strip (2517) with cleaning solution by depositing cleaning solution from a bottle or other container (not shown) onto gauze strip (2517).

To use assembly (2510) to remove debris from and apply lubricant to end effector (180), the operator would first load sponge (2518) with lubricant (L) by inserting rod (2582) of plunger (2512) into bore (2572) of capsule (2514) and advancing rod (2582) distally from the proximal state shown in FIGS. 46A and 47A to the distal state shown in FIGS. 46B and 47B such that lubricant (L) is directed through outlets (2575) and soaked into sponge (2518).

In cases where end effector (180) is laparoscopically disposed within an anatomical cavity of the patient, the operator may position assembly (2510) and a surgical grasper (G) such that grasper (G) is aligned with grip (2580) of plunger (2512), advance grasper (G) distally as shown in FIG. 46C (and/or advance assembly (2510) proximally), and close grasper (G) onto grip (2580) to securely hold assembly (2510) via grasper (G). The operator may then use grasper (G) to advance assembly (2510) distally into the anatomical cavity of the patient through trocar (T), as shown in FIG. 46D.

With assembly (2510) held by grasper (G) within the anatomical cavity of the patient, end effector (180) may be advanced generally orthogonally to assembly (2510) to position gauze strip (2517) between electrode surfaces (194, 196). Closure trigger (126) of instrument (100) is then squeezed by the operator to close end effector jaws (182, 184) on gauze strip (2517), thereby engaging electrode surfaces (194, 196) with cleaning surface (2544), as shown in FIG. 46E. In some versions, assembly (2510) may be rotated via rotation of grasper (G) about its longitudinal axis while end effector jaws (182, 184) are closed on gauze strip (2517) and thereby scrub electrode surfaces (194, 196) along cleaning surface (2544) for dislodging debris from end effector (180). In addition, or alternatively, the operator may press activation button (130) to selectively activate electrode surfaces (194, 196) of jaws (182, 184) while closed on gauze strip (2517) to heat up any debris on end effector (180) to assist with dislodging such debris from end effector (180). End effector jaws (182, 184) may be disengaged from gauze strip (2517) following sufficient cleaning of electrode surfaces (194, 196) and the side surfaces of jaws (182, 184).

With assembly (2510) continuing to be held by grasper (G) within the anatomical cavity of the patient, end effector (180) may be slid distally along assembly (2510) to position sponge (2518) between electrode surfaces (194, 196). Closure trigger (126) of instrument (100) is then squeezed by the operator to close end effector jaws (182, 184) on sponge (2518), thereby engaging electrode surfaces (194, 196) with application surface (2524) such that at least some lubricant is expelled from sponge (2518) onto each electrode surface (194, 196) and onto each side surface of jaws (182, 184), as shown in FIG. 46F. In any event, end effector jaws (182, 184) may be disengaged from sponge (2518) while lubricant remains applied to electrode surfaces (194, 196) and the side surfaces of jaws (182, 184), and assembly (2510) may be retracted proximally out of the patient's anatomical cavity through trocar (T). In cases where end effector (180) is disposed within an anatomical cavity of the patient in an open procedure or where end effector (180) is disposed external to the patient, it will be appreciated that the above steps may be performed without the use of grasper (G). For example, the operator may manipulate assembly (2510) by manually grasping grip (2580) with the operator's hand in place of grasper (G).
X. Exemplary Lubricant Applier Bottle

FIG. 48 shows another exemplary lubricant applicator in the form of a lubricant applier bottle (2610) that may be used to apply lubricant to end effector (180). Bottle (2610) of this example comprises an openable end (2612) and a closed end (2614) opposite each other. Bottle (2610) further includes a body (2616a) which presents openable end (2612) and a cap (2616b) which selectively covers openable end (2612) for transitioning openable end (2612) between a closed state and the illustrated opened state. For example, body (2616a) and cap (2616b) may be configured to be threadably coupled to each other. A lubricant application member in the form of a sponge (2618) is secured to the bottom end of a rigid stem (2616c) extending downwardly from cap (2616b). A lubricant reservoir in the form of an internal compartment (2627) is defined within body (2616a) between closed end (2614) openable end (2612). In the example shown, compartment (2627) is configured to receive sponge (2618) when cap (2616b) is coupled to body (2616a) such that openable end (2612) is in the closed state. In any event, one or more application surfaces (2624) of sponge (2618) are exposed outside of compartment (2627) when openable end (2612) is in the opened state with cap (2616b) removed from body (2616a) and sponge (2618) withdrawn therefrom for applying lubricant to electrode surfaces (194, 196).

In this regard, compartment (2627) may be pre-loaded (e.g., pre-filled) with lubricant, at least some of which may be soaked into sponge (2618), such that an operator may transition openable end (2612) to the open state and immediately use cap (2616b) of bottle (2610) to apply lubricant to end effector (180) without needing to first load sponge (2618) with lubricant.

While not shown, bottle (2610) may include an ultraviolet light (UV) emitting device, such as a black light lamp or an ultraviolet light emitting diode (LED), to enable the operator to visually discern whether a sufficient amount of lubricant has been uniformly applied to the desired surfaces of end effector (180), or whether further application or removal of lubricant may be desired, at least in cases where the lubricant is UV-sensitive.

Y. Exemplary Cleaning Solution Applier Bottle

FIG. 49 shows an exemplary cleaning solution applicator in the form of a cleaning solution applier bottle (2710) that may be used to clean end effector (180). Bottle (2710) of this example comprises an openable end (2712) and a closed end (2714) opposite each other. Bottle (2710) further includes a body (2716a) which presents openable end (2712) and a cap (2716b) which selectively covers openable end (2712) for transitioning openable end (2712) between a closed state and the illustrated opened state. For example, body (2716a) and cap (2716b) may be configured to be threadably coupled to each other. A pre-application jaw cleaning member in the form of a brush defined by a plurality of bristles (2717) is secured to the bottom end of a rigid stem (2716c) extending downwardly from cap (2716b). A cleaning solution reservoir in the form of an internal compartment (2727) is defined within body (2716a) between closed end (2714) openable end (2712). In the example shown, compartment (2727) is configured to receive bristles (2717) when cap (2716b) is coupled to body (2716a) such that openable end (2712) is in the closed state. In any event, at least some bristles (2717) are exposed outside of compartment (2727) when openable end (2712) is in the opened state with cap (2716b) removed from body (2716a) and the brush withdrawn therefrom for cleaning electrode surfaces (194, 196).

In this regard, compartment (2727) may be pre-loaded (e.g., pre-filled) with cleaning solution, at least some of which may be soaked into bristles (2717), such that an operator may transition openable end (2712) to the open state and immediately use cap (2716b) of bottle (2710) to clean end effector (180) without needing to first load bristles (2717) with cleaning solution.

In some versions, cleaning solution applier bottle (2710) may be provided together with lubricant applier bottle (2610) described above to collectively define a lubricant applier kit.
III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus configured for use with an end effector of a surgical instrument, comprising: (a) a housing, wherein the housing includes a first housing portion defining a first gap, wherein the first gap is configured to receive a jaw of the end effector; and (b) a lubricant application member positioned within the first gap, wherein the lubricant application member is configured to be loaded with a lubricant for applying the lubricant to a tissue clamping surface of the jaw when the jaw is received within the first gap.

### Example 2

The apparatus of Example 1, wherein the first housing portion defines a U shape to present the first gap.

### Example 3

The apparatus of any one or more of Examples 1 through 2, wherein the lubricant application member includes at least one ridge configured to contact at least one side surface of the jaw when the jaw is received within the first gap for applying the lubricant to the at least one side surface of the jaw.

### Example 4

The apparatus of any one or more of Examples 1 through 3, wherein the housing is rigid.

### Example 5

The apparatus of any one or more of Examples 1 through 4, wherein the lubricant application member includes a sponge.

### Example 6

The apparatus of Example 5, wherein the sponge includes a polymeric foam.

### Example 7

The apparatus of any one or more of Examples 1 through 6, wherein the lubricant application member includes a pair of opposed lubricant application surfaces, wherein each of the pair of opposed lubricant application surface is exposed within the first gap.

### Example 8

The apparatus of any one or more of Examples 1 through 7, further comprising a lubricant discharge member, wherein the lubricant discharge member is configured to selectively direct the lubricant from a lubricant reservoir to the lubricant application member.

### Example 9

The apparatus of Example 8, wherein the lubricant discharge member includes at least one of a button, a pump bulb, or a paddle.

### Example 10

The apparatus of any one or more of Examples 1 through 9, further comprising an ultraviolet light (UV) emitting device secured to the housing.

### Example 11

The apparatus of any one or more of Examples 1 through 10, wherein the housing further includes a second housing portion defining a second gap, wherein the second gap is configured to receive the jaw of the end effector.

### Example 12

The apparatus of Example 11, further comprising an excess lubricant removal member positioned within the second gap, wherein the excess lubricant removal member is configured to remove excess lubricant from the tissue clamping surface of the jaw when the jaw is received within the second gap.

### Example 13

The apparatus of Example 12, wherein the excess lubricant removal member includes at least one of a sponge or a cloth.

### Example 14

The apparatus of Example 11, further comprising a jaw cleaning member positioned within the second gap, wherein the jaw cleaning member is configured to remove debris from the tissue clamping surface of the jaw when the jaw is received within the second gap.

### Example 15

The apparatus of Example 14, wherein the jaw cleaning member includes at least one of an abrasive, a brush, or a gauze strip.

### Example 16

An apparatus configured for use with an end effector of a surgical instrument, comprising: (a) a housing defining a first gap, wherein the first gap is configured to receive a jaw of the end effector; (b) a lubricant application member positioned within the first gap; and (c) a lubricant carried by the lubricant application member.

### Example 17

The apparatus of Example 16, wherein the housing defines a second gap opposite the first gap, wherein the second gap is configured to receive the jaw of the end effector.

### Example 18

The apparatus of Example 17, further comprising one of an excess lubricant removal member or a jaw cleaning member positioned within the second gap.

### Example 19

An apparatus configured for use with an end effector of a surgical instrument, comprising: (a) a rigid support member; and (b) a lubricant application member secured to the rigid support member, wherein the lubricant application member is configured to confront a jaw of the end effector, wherein the lubricant application member is configured to be loaded with a lubricant for applying the lubricant to a tissue clamping surface of the jaw when the jaw is confronted by the lubricant application member.

### Example 20

A multi-pack comprising a plurality of the apparatuses of Example 19, wherein each apparatus of the plurality of apparatuses is releasably coupled relative to the remaining apparatuses of the plurality of apparatuses.

### Example 21

An apparatus configured for use with an end effector of a surgical instrument, comprising: (a) a jaw cleaning member configured to remove debris from the end effector; and (b) a lubricant application member configured to be loaded with a lubricant for applying the lubricant to the end effector, wherein the jaw cleaning member and the lubricant application member are configured to be inserted together into an anatomical cavity of a patient through a trocar.

### Example 22

The apparatus of Example 21, further comprising a capsule, wherein the jaw cleaning member and the lubricant application member are each presented by the capsule.

### Example 23

The apparatus of Example 22, wherein the capsule includes a rigid base, wherein the jaw cleaning member and the lubricant application member are each wrapped around respective portions of the rigid base.

### Example 24

The apparatus of Example 23, wherein the rigid base includes silicone.

### Example 25

The apparatus of any one or more of Examples 22 through 23, wherein the capsule includes a lubricant reservoir, wherein the lubricant application member is configured to be loaded with the lubricant from the lubricant reservoir.

### Example 26

The apparatus of Example 25, wherein the lubricant reservoir includes a rupturable pod.

### Example 27

The apparatus of Example 26, wherein the capsule includes a bore, wherein the rupturable pod is positioned within the bore.

### Example 28

The apparatus of Example 27, wherein the capsule includes at least one outlet extending between the bore and the lubricant application member.

### Example 29

The apparatus of any one or more of Examples 27 through 28, further comprising a plunger having a rod, wherein the rod is received within the bore, wherein the rod is movable between a first position in which the rod is spaced apart from the rupturable pod and a second position in which the rod ruptures the rupturable pod to load the lubricant from the rupturable pod into the lubricant application member.

### Example 30

The apparatus of Example 29, wherein the plunger is configured to be secured to the capsule via a snap-fit when the rod is in the second position.

### Example 31

The apparatus of any one or more of Examples 29 through 30, wherein the plunger further has a grip configured to be held by a surgical grasper.

### Example 32

The apparatus of Example 31, wherein the grip is proximal of the rod.

### Example 33

The apparatus of any one or more of Examples 21 through 32, wherein the lubricant application member includes a sponge.

### Example 34

The apparatus of Example 33, wherein the sponge includes a polymeric foam.

### Example 35

The apparatus of any one or more of Examples 21 through 34, wherein the jaw cleaning member includes at least one of an abrasive, a brush, or a gauze strip.

### Example 36

An apparatus configured for use with an end effector of a surgical instrument, comprising: (a) a capsule including a lubricant application member configured to be loaded with a lubricant for applying the lubricant to the end effector; and (b) a plunger configured to discharge the lubricant from a lubricant reservoir to the lubricant application member.

### Example 37

The apparatus of Example 36, wherein the capsule and the plunger are configured to be inserted together into an anatomical cavity of a patient through a trocar.

### Example 38

The apparatus of any one or more of Examples 36 through 37, wherein the plunger is configured to be secured to the capsule via a snap-fit.

### Example 39

An apparatus configured for use with an end effector of a surgical instrument, comprising: (a) a capsule including: (i) a rigid base including a bore, (ii) a lubricant reservoir positioned within the bore and containing a lubricant, and (iii) a lubricant application member wrapped around a first portion of the base and configured to be loaded with the lubricant from the lubricant reservoir for applying the lubricant to the end effector; and (b) a plunger configured to be selectively secured to the capsule, the plunger including: (i) a rod configured to be inserted into the bore to discharge the lubricant from the lubricant reservoir to the lubricant application member, and (ii) a grip configured to be held by a surgical grasper.

### Example 40

The apparatus of Example 39, wherein the capsule further includes a jaw cleaning member wrapped around a second portion of the rigid base and configured to remove debris from the end effector.

### Example 41

An apparatus configured for use with an end effector of a surgical instrument, comprising: (a) a flexible housing defining an openable end, wherein the openable end is configured to receive a jaw of the end effector when the openable end is in an opened state; (b) a lubricant reservoir positioned within the flexible housing; and (c) a lubricant application member extending from the lubricant reservoir toward the openable end and configured to be loaded with the lubricant from the lubricant reservoir for applying the lubricant to the jaw.

### Example 42

The apparatus of Example 41, further comprising a partition positioned between the lubricant reservoir and the openable end and configured to prevent the jaw from entering the lubricant reservoir.

### Example 43

An apparatus configured for use with an end effector of a surgical instrument, comprising: (a) a housing; (b) a first well extending through a portion of the housing and configured to receive a jaw of the end effector; and (c) a lubricant application member extending across the first well and configured to be loaded with a lubricant for applying the lubricant to the jaw.

### Example 44

The apparatus of Example 43, further comprising (a) a second well extending through a portion of the housing and configured to receive the jaw of the end effector; and (b) a jaw cleaning member extending across the second well and configured to remove debris from the jaw.

### Example 45

An apparatus configured for use with an end effector of a surgical instrument, comprising: (a) a package including a plurality of pockets; and (b) a plurality of lubricant application members, wherein each lubricant application member of the plurality of lubricant application members is loaded with a lubricant for applying the lubricant to the end effector, wherein each lubricant application member of the plurality of lubricant application members is sealed within a respective pocket of the plurality of pockets.

### Example 46

An apparatus configured for use with an end effector of a surgical instrument, comprising: (a) a lubricant reservoir containing a lubricant; and (b) a manifold having (i) a plurality of lubricant application surfaces configured to confront respective tissue clamping surfaces of the end effector, and (ii) a plurality of channels extending from the lubricant reservoir to corresponding lubricant application surfaces of the plurality of lubricant application surfaces for directing the lubricant from the lubricant reservoir to the respective tissue clamping surfaces.

### Example 47

The apparatus of Example 46, wherein the manifold is triangular.

### Example 48

The apparatus of any one or more of Examples 46 through 47, wherein each lubricant application surface of the plurality of lubricant application surfaces is configured to confront the respective tissue clamping surface when the end effector is in an open state.

### Example 49

The apparatus of any one or more of Examples 46 through 48, wherein the manifold further has at least one rail configured to engage at least one knife pathway portion of the end effector to provide a gap between each lubricant application surface of the plurality of lubricant application surfaces and the respective tissue clamping surface.

### Example 50

The apparatus of any one or more of Examples 46 through 49, wherein the manifold further has a jaw cleaning surface configured to remove debris from each of the tissue clamping surfaces.

### Example 51

An apparatus configured for use with an end effector of a surgical instrument, comprising: (a) a housing extending in a longitudinal direction, wherein the housing includes: (i) a laterally-opposed pair of side surfaces facing each other, and (ii) a distal surface facing proximally, wherein the side surfaces and the distal surface collectively define a gap, wherein the gap is configured to receive a jaw of the end effector such that the side surfaces confront respective lateral sides of the jaw and such that the distal surface confronts a distal tip of the jaw; and (b) a lubricant application member positioned within the gap, wherein the lubricant application member is configured to be loaded with a lubricant for applying the lubricant to a tissue clamping surface of the jaw when the jaw is received within the gap.

### Example 52

A multi-pack comprising a plurality of the apparatuses of Example 51, wherein each apparatus of the plurality of apparatuses is releasably coupled relative to the remaining apparatuses of the plurality of apparatuses.

IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Additionally, any one or more of the teachings herein may be combined with any one or more of the teachings of U.S. Pat. App. No. [Atty. Ref. END9436USNP1], entitled “Container Features for Surgical Instrument Lubricant,” filed on even date herewith, the disclosure of which is incorporated by reference herein.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus configured for use with an end effector of a surgical instrument, comprising:
(a) a housing, wherein the housing includes a first housing portion defining a first gap, wherein the first gap is configured to receive a jaw of the end effector; and
(b) a lubricant application member positioned within the first gap, wherein the lubricant application member is configured to be loaded with a lubricant for applying the lubricant to a tissue clamping surface of the jaw when the jaw is received within the first gap.

2. The apparatus of claim 1, wherein the first housing portion defines a U shape to present the first gap.

3. The apparatus of claim 1, wherein the lubricant application member includes at least one ridge configured to contact at least one side surface of the jaw when the jaw is received within the first gap for applying the lubricant to the at least one side surface of the jaw.

4. The apparatus of claim 1, wherein the housing is rigid.

5. The apparatus of claim 1, wherein the lubricant application member includes a sponge.

6. The apparatus of claim 5, wherein the sponge includes a polymeric foam.

7. The apparatus of claim 1, wherein the lubricant application member includes a pair of opposed lubricant application surfaces, wherein each of the pair of opposed lubricant application surface is exposed within the first gap.

8. The apparatus of claim 1, further comprising a lubricant discharge member, wherein the lubricant discharge member is configured to selectively direct the lubricant from a lubricant reservoir to the lubricant application member.

9. The apparatus of claim 8, wherein the lubricant discharge member includes at least one of a button, a pump bulb, or a paddle.

10. The apparatus of claim 1, further comprising an ultraviolet light (UV) emitting device secured to the housing.

11. The apparatus of claim 1, wherein the housing further includes a second housing portion defining a second gap, wherein the second gap is configured to receive the jaw of the end effector.

12. The apparatus of claim 11, further comprising an excess lubricant removal member positioned within the second gap, wherein the excess lubricant removal member is configured to remove excess lubricant from the tissue clamping surface of the jaw when the jaw is received within the second gap.

13. The apparatus of claim 12, wherein the excess lubricant removal member includes at least one of a sponge or a cloth.

14. The apparatus of claim 11, further comprising a jaw cleaning member positioned within the second gap, wherein the jaw cleaning member is configured to remove debris from the tissue clamping surface of the jaw when the jaw is received within the second gap.

15. The apparatus of claim 14, wherein the jaw cleaning member includes at least one of an abrasive, a brush, or a gauze strip.

16. An apparatus configured for use with an end effector of a surgical instrument, comprising:
(a) a housing defining a first gap, wherein the first gap is configured to receive a jaw of the end effector;
(b) a lubricant application member positioned within the first gap; and
(c) a lubricant carried by the lubricant application member.

17. The apparatus of claim 16, wherein the housing defines a second gap opposite the first gap, wherein the second gap is configured to receive the jaw of the end effector.

18. The apparatus of claim 17, further comprising one of an excess lubricant removal member or a jaw cleaning member positioned within the second gap.

19. An apparatus configured for use with an end effector of a surgical instrument, comprising:
(a) a rigid support member; and
(b) a lubricant application member secured to the rigid support member, wherein the lubricant application member is configured to confront a jaw of the end effector, wherein the lubricant application member is configured to be loaded with a lubricant for applying the lubricant to a tissue clamping surface of the jaw when the jaw is confronted by the lubricant application member.

20. A multi-pack comprising a plurality of the apparatuses of claim 19, wherein each apparatus of the plurality of apparatuses is releasably coupled relative to the remaining apparatuses of the plurality of apparatuses.
